(19) United States
(12) Patent Application Publication
(10) Pub. No.: US 2024/0155868 A1
(43) Pub. Date: May 9, 2024

(54) CONDENSED CYCLIC COMPOUND, ORGANIC LIGHT-RECEIVING DEVICE INCLUDING THE SAME, AND ELECTRONIC APPARATUS AND ELECTRONIC DEVICE INCLUDING THE LIGHT-RECEIVING DEVICE

(71) Applicant: Samsung Display Co., Ltd., Yongin-si (KR)

(72) Inventors: Hyejin Jung, Yongin-si (KR); Hwasook Ryu, Yongin-si (KR); Seokgyu Yoon, Yongin-si (KR)

(21) Appl. No.: 18/472,992

(22) Filed: Sep. 22, 2023

(30) Foreign Application Priority Data
Sep. 30, 2022 (KR) 10-2022-0125778

(51) Int. Cl. H10K 50/86; C07D 421/04; C07D 421/14; C07D 471/04; C07D 487/04; C07D 495/04; C07D 495/14; C07D 513/04; C07D 517/14; C07F 7/08; H10K 50/15; H10K 50/16; H10K 85/60

(52) U.S. Cl. CPC H10K 50/865; C07D 421/04; C07D 421/14; C07D 471/04; C07D 487/04; C07D 495/04; C07D 495/14; C07D 513/04; C07D 517/14; C07F 7/0812; H10K 50/15; H10K 50/16; H10K 85/655; H10K 85/657

(57) ABSTRACT

A condensed cyclic compound includes at least one first repeating unit represented by Formula 1-1 and/or Formula 1-2 and at least one second repeating unit represented by Formula 2-1 and/or Formula 2-2. A light-receiving device includes the condensed cyclic compound in a light-receiving layer of the light-receiving device, and an electronic apparatus and an electronic device include the light-receiving device.

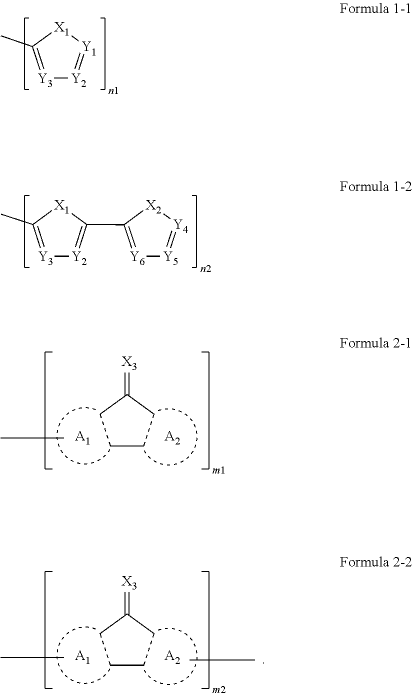

### CROSS-REFERENCE TO RELATED APPLICATION

This application claims priority to and the benefit of Korean Patent Application No. 10-2022-0125778, filed on Sep. 30, 2022, in the Korean Intellectual Property Office, the content of which is incorporated by reference herein in its entirety.

### BACKGROUND

### 1. Field

One or more embodiments of the present disclosure relate to a condensed cyclic compound, a light-receiving device including the condensed cyclic compound, and an electronic apparatus and an electronic device that include the light-receiving device.

### 2. Description of the Related Art

Organic light-receiving devices, which are devices including organic compounds capable of being excited by light, are widely utilized in digital cameras, broadcasting cameras, surveillance cameras, computer image cameras, camcorders, automotive sensors, household sensors, solar cells, and/or the like.

A light-receiving device may have a structure in which a first electrode is arranged on a substrate, and an interlayer and a second electrode are sequentially formed on the first electrode. The interlayer may include an electron-donating layer including an electron donor and an electron-accepting layer including an electron acceptor. A p-type or kind semiconductor material may be utilized as the electron donor, and an n-type or kind semiconductor material may be utilized as the electron acceptor.

When light is irradiated to the organic light-receiving device, electrons may be excited and holes may be generated due to absorption of the light, and the excited electrons and the newly generated holes may be paired to form excitons. The excitons move to an interface of the interlayer and are separated into electrons and holes again according to characteristics of the interface. The separated electrons and holes move to each corresponding electrode, thereby generating current.

### SUMMARY

One or more aspects of embodiments of the present disclosure are directed toward a condensed cyclic compound, a light-receiving device having characteristics of high sensitivity, high efficiency, and long lifespan by including the condensed cyclic compound, and an electronic apparatus and an electronic device including the light-receiving device.

Additional aspects will be set forth in part in the description which follows and, in part, will be apparent from the description, or may be learned by practice of the presented embodiments of the disclosure.

According to one or more embodiments of the present disclosure, a condensed cyclic compound may include at least one first repeating unit and at least one second repeating unit, wherein the first repeating unit may include at least one selected from a first-first moiety represented by Formula 1-1 and a first-second moiety represented by Formula 1-2, the second repeating unit may include at least one selected from a second-first moiety represented by Formula 2-1 and a second-second moiety represented by Formula 2-2, and at least one of the first repeating unit may further include at least one π electron-rich C₁-C₆₀ group.

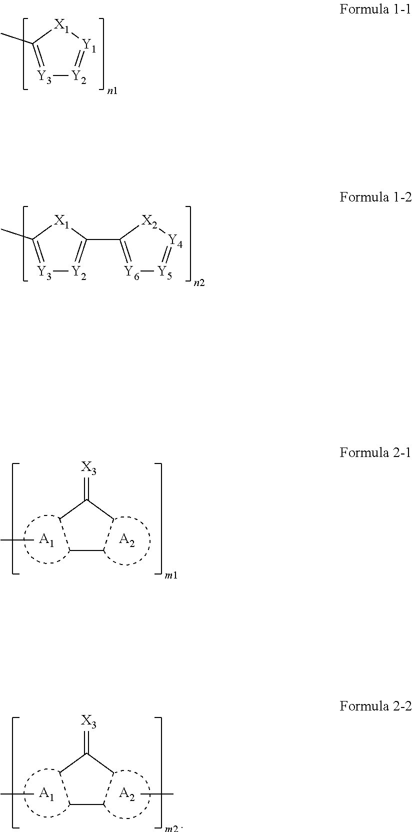

In Formulae 1-1 and 1-2,
X₁ and X₂ may each independently be O, S, or Se,
Y₁ may be C(R₁), CH, or C—*,
Y₂ may be C(R₂) or CH,
Y₃ may be C(R₃) or CH,
Y₄ may be C(R₄), CH, or C—*,
Y₅ may be C(R₅) or CH,
Y₆ may be C(R₆) or CH,
R₁ and R₂, R₂ and R₃, R₄ and R₅, R₅ and R₆, or R₂ and R₆ may optionally be bonded to each other to form a C₃-C₆₀ carbocyclic group unsubstituted or substituted with at least one R₁₀ₐ, a C₁-C₆₀ heterocyclic group unsubstituted or substituted with at least one R₁₀ₐ, or a π electron-rich C₁-C₆₀ group unsubstituted or substituted with at least one R₁₀ₐ,
n1 and n2 may each independently be an integer from 0 to 2, and the sum of
n1 and n2 may be 1 or more,
when n1 is 0, the first-first moiety may not be included,
when n2 is 0, the first-second moiety may not be included,
R₁ to R₆ and R₁₀ₐ may each independently be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a π electron-rich C₁-C₆₀ group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁₁ to Q₁₃, Q₂₁ to Q₂₃, and Q₃₁ to Q₃₃ may each independently be hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, or a π electron-rich C₁-C₆₀ group, each unsubstituted or substituted with deuterium, —F, a cyano group, a C₁-C₆₀ alkyl group, a C₁-C₆₀ alkoxy group, a phenyl group, a biphenyl group, or any combination thereof,
two neighboring substituents selected from among Q₁₁ to Q₁₃, Q₂₁ to Q₂₃, and Q₃₁ to Q₃₃ may optionally be bonded to each other to form a C₃-C₆₀ carbocyclic group unsubstituted or substituted with at least one R₁₀ₐ, a C₁-C₆₀ heterocyclic group unsubstituted or substituted with at least one R₁₀ₐ, or a π electron-rich C₁-C₆₀ group unsubstituted or substituted with at least one R₁₀ₐ, and
* may indicate a binding site to a neighboring repeating unit.

In Formulae 2-1 and 2-2,
X₃ may be C(R₇)(R₈), C(R₇)(H), or N(R₇),
ring A₁ and ring A₂ may each independently include a C₃-C₆₀ carbocyclic group unsubstituted or substituted with at least one R_{10b} or a C₁-C₆₀ heterocyclic group unsubstituted or substituted with at least one R_{10b},
m1 and m2 may each independently be an integer from 0 to 2, and the sum of m1 and m2 may be 1 or more,
when m1 is 0, the second-first moiety may not be included,
when m2 is 0, the second-second moiety may not be included,
R₇, R₈, and R_{10b} may each independently be
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₄₁)(Q₄₂)(Q₄₃), —N(Q₄₁)(Q₄₂), —B(Q₄₁)(Q₄₂), —C(═O)(Q₄₁), —S(═O)₂(Q₄₁), —P(═O)(Q₄₁)(Q₄₂), or any combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₅₁)(Q₅₂)(Q₅₃), —N(Q₅₁)(Q₅₂), —B(Q₅₁)(Q₅₂), —C(═O)(Q₅₁), —S(═O)₂(Q₅₁), —P(═O)(Q₅₁)(Q₅₂), or any combination thereof; or
—Si(Q₆₁)(Q₆₂)(Q₆₃), —N(Q₆₁)(Q₆₂), —B(Q₆₁)(Q₆₂), —C(═O)(Q₆₁), —S(═O)₂(Q₆₁), or —P(═O)(Q₆₁)(Q₆₂),
Q₄₁ to Q₄₃, Q₅₁ to Q₅₃, and Q₆₁ to Q₆₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
two neighboring substituents selected from among Q₄₁ to Q₄₃, Q₅₁ to Q₅₃, and Q₆₁ to Q₆₃ may optionally be bonded to each other to form a C₃-C₆₀ carbocyclic group unsubstituted or substituted with at least one R_{10b} or a C₁-C₆₀ heterocyclic group unsubstituted or substituted with at least one R_{10b}.

According to one or more embodiments of the present disclosure, a light-receiving device may include the condensed cyclic compound of the present disclosure.

According to one or more embodiments of the present disclosure, an electronic apparatus includes the light-receiving device.

According to one or more embodiments of the present disclosure, an electronic device includes the light-receiving device.

### BRIEF DESCRIPTION OF THE DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIG. 1 is a schematic view of a structure of a light-receiving device according to one or more embodiments of the present disclosure;

FIG. 2 is a schematic view of a structure of an electronic apparatus according to one or more embodiments of the present disclosure;

FIG. 3 is a schematic view of a structure of an electronic apparatus according to one or more embodiments of the present disclosure; and

FIG. 4 is a schematic perspective of an electronic device according to one or more embodiments of the present disclosure.

### DETAILED DESCRIPTION

Reference will now be made in more detail to embodiments, examples of which are illustrated in the accompanying drawings, wherein like reference numerals refer to like elements throughout the present disclosure, and duplicative descriptions thereof may not be provided for conciseness. In this regard, the embodiments of the present disclosure may have different forms and should not be construed as being limited to the descriptions set forth herein. Accordingly, the embodiments of the present disclosure are merely described, by referring to the drawings, to explain aspects of the present disclosure. As utilized herein, the term “and/or” or “or” may include any and all combinations of one or more of the associated listed items. Throughout the disclosure, the expression “at least one of a, b, or c” indicates only a, only b, only c, both (e.g., simultaneously) a and b, both (e.g., simultaneously) a and c, both (e.g., simultaneously) b and c, all of a, b, and c, or variations thereof.

As the present disclosure allows for one or more suitable changes and numerous embodiments, particular embodiments will be illustrated in the drawings and described in more detail in the written description. Effects and features of the present disclosure, and methods of achieving the same will be clarified by referring to embodiments described in more detail with reference to the drawings. The present disclosure may, however, be embodied in many different forms and should not be construed as being limited to the embodiments set forth herein.

Hereinafter, embodiments of the disclosure will be described in more detail with reference to the accompanying drawings. The same or corresponding components will be denoted by the same reference numerals, and thus redundant description thereof will not be provided for conciseness.

It will be understood that although the terms “first,” “second,” and/or the like may be utilized herein to describe one or more suitable components, these components should not be limited by these terms. These terms are only utilized to distinguish one component from another.

An expression utilized in the singular encompasses the expression of the plural, unless it has a clearly different meaning in the context.

It will be further understood that the terms “comprise(s)”, “include(s)”, “have/has”, and/or the like as utilized herein specify the presence of stated features or components, but do not preclude the presence or addition of one or more other features or components.

Sizes of elements in the drawings may be exaggerated for convenience of explanation. In other words, because sizes and thicknesses of components in the drawings are arbitrarily illustrated for convenience of explanation, the following embodiments are not limited thereto.

When a certain embodiment is implemented differently, a specific process order may be performed differently from the described order. For example, two processes described in succession may be performed substantially concurrently (e.g., simultaneously), or may be performed in an order opposite to the described order.

It will be understood that when a layer, region, or component is referred to as being “connected to” another layer, region, or component, the layer, region, or component may be directly connected to the another layer, region, or component, or indirectly connected to the another layer, region, or component as an intervening layer, region, or component may be present. For example, it will be understood that when a layer, region, or component is referred to as being “electrically connected to” another layer, region, or component, the layer, region, or component may be directly electrically connected to the another layer, region, or component, or indirectly electrically connected to the another layer, region, or component as an intervening layer, region, or component may be present.

A condensed cyclic compound according to one or more embodiments may include at least one first repeating unit and at least one second repeating unit, wherein the first repeating unit may include at least one selected from among a first-first moiety represented by Formula 1-1 and a first-second moiety represented by Formula 1-2, the second repeating unit may include at least one selected from among a second-first moiety represented by Formula 2-1 and a second-second moiety represented by Formula 2-2, and at least one of the first repeating unit may further include at least one π electron-rich C₁-C₆₀ group:

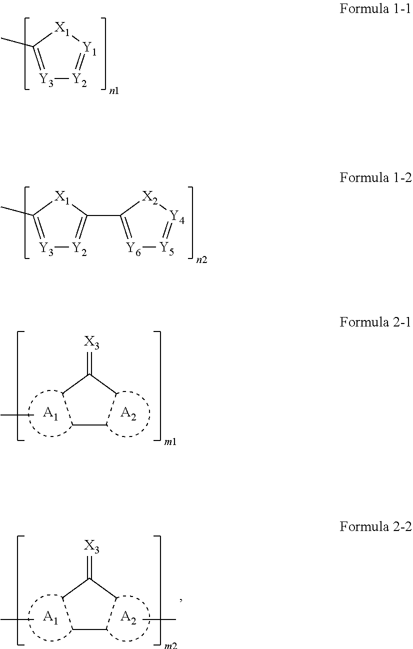

wherein, in Formulae 1-1 and 1-2,
X₁ and X₂ may each independently be O, S, or Se,
Y₁ may be C(R₁), CH, or C—*,
Y₂ may be C(R₂) or CH,
Y₃ may be C(R₃) or CH,
Y₄ may be C(R₄), CH, or C—*,
Y₅ may be C(R₅) or CH,
Y₆ may be C(R₆) or CH,
R₁ and R₂, R₂ and R₃, R₄ and R₅, R₅ and R₆, or R₂ and R₆ may optionally be bonded to each other to form: a C₃-C₆₀ carbocyclic group unsubstituted or substituted with at least one R₁₀ₐ; a C₁-C₆₀ heterocyclic group unsubstituted or substituted with at least one R₁₀ₐ; or a π electron-rich C₁-C₆₀ group unsubstituted or substituted with at least one R₁₀ₐ,
n1 and n2 may each independently be an integer from 0 to 2, and the sum of n1 and n2 may be 1 or more,
when n1 is 0, the first-first moiety may not be included,
when n2 is 0, the first-second moiety may not be included,
R₁ to R₆ and R₁₀ₐ may each independently be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a π electron-rich C₁-C₆₀ group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, or a π electron-rich C₁-C₆₀ group, each unsubstituted or substituted with deuterium, —F, a cyano group, a C₁-C₆₀ alkyl group, a C₁-C₆₀ alkoxy group, a phenyl group, a biphenyl group, or any combination thereof,
two neighboring substituents selected from among Q₁₁ to Q₁₃, Q₂₁ to Q₂₃, and Q₃₁ to Q₃₃ may optionally be bonded to each other to form: a C₃-C₆₀ carbocyclic group unsubstituted or substituted with at least one R₁₀ₐ; a C₁-C₆₀ heterocyclic group unsubstituted or substituted with at least one R₁₀ₐ; or a π electron-rich C₁-C₆₀ group unsubstituted or substituted with at least one R₁₀ₐ, and
* may indicate a binding site to a neighboring repeating unit, and
wherein, in Formulae 2-1 and 2-2,
X₃ may be C(R₇)(R₈), C(R₇)(H), or N(R₇),
ring A₁ and ring A₂ may each independently include: a C₃-C₆₀ carbocyclic group unsubstituted or substituted with at least one R_{10b}; or a C₁-C₆₀ heterocyclic group unsubstituted or substituted with at least one R_{10b},
m1 and m2 may each independently be an integer from 0 to 2, and the sum of m1 and m2 may be 1 or more,
when m1 is 0, the second-first moiety may not be included,
when m2 is 0, the second-second moiety may not be included,
R₇, R₈, and R_{10b} may each independently be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₄₁)(Q₄₂)(Q₄₃), —N(Q₄₁)(Q₄₂), —B(Q₄₁)(Q₄₂), —C(═O)(Q₄₁), —S(═O)₂(Q₄₁), —P(═O)(Q₄₁)(Q₄₂), or any combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₅₁)(Q₅₂)(Q₅₃), —N(Q₅₁)(Q₅₂), —B(Q₅₁)(Q₅₂), —C(═O)(Q₅₁), —S(═O)₂(Q₅₁), —P(═O)(Q₅₁)(Q₅₂), or any combination thereof; or
—Si(Q₆₁)(Q₆₂)(Q₆₃), —N(Q₆₁)(Q₆₂), —B(Q₆₁)(Q₆₂), —C(═O)(Q₆₁), —S(═O)₂(Q₆₁), or —P(═O)(Q₆₁)(Q₆₂),
Q₄₁ to Q₄₃, Q₅₁ to Q₅₃, and Q₆₁ to Q₆₃ may each independently be: hydrogen; deuterium; —F; —CI; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
two neighboring substituents selected from among Q₄₁ to Q₄₃, Q₅₁ to Q₅₃, and Q₆₁ to Q₆₃ may optionally be bonded to each other to form: a C₃-C₆₀ carbocyclic group unsubstituted or substituted with at least one R_{10b}; or a C₁-C₆₀ heterocyclic group unsubstituted or substituted with at least one R_{10b}.

In the condensed cyclic compound according to one or more embodiments,
n1 may be 1, and n2 may be 0, and
one selected from among Conditions 1A to 1C may be satisfied:
1A) m1 is 1, and m2 is 0;
1B) m1 is 2, and m2 is 0; and
1C) m1 is 1, and m2 is 1.

The condensed cyclic compound according to one or more embodiments may satisfy Condition 1A, and may be represented by Formula 1-A:

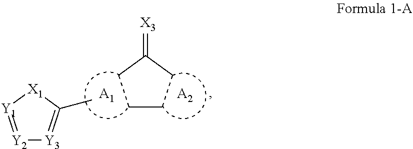

wherein, in Formula 1-A,
X₁, X₃, Y₁ to Y₃, ring A₁, and ring A₂ may each be the same as described herein.

The condensed cyclic compound according to one or more embodiments may satisfy Condition 1B, and may be represented by Formula 1-B:

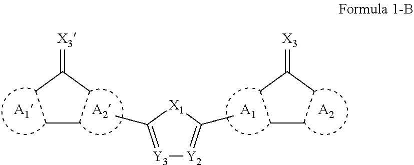

wherein, in Formula 1-B,
X₃′ may correspond to X₃, and may be identical to or different from X₃,
ring A₁′ may correspond to ring A₁, and may be identical to or different from ring A₁,
ring A₂′ may correspond to ring A₂, and may be identical to or different from ring A₂, and
X₁, X₃, Y₁ to Y₃, ring A₁, and ring A₂ may each be the same as described herein.

The condensed cyclic compound according to one or more embodiments may satisfy Condition 1C, and may be represented by Formula 1-C:

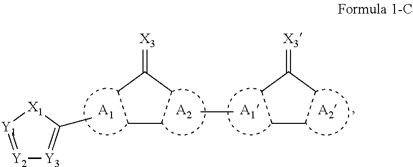

wherein, in Formula 1-C,
X₃′ may correspond to X₃, and may be identical to or different from X₃,
ring A₁′ may correspond to ring A₁, and may be identical to or different from ring A₁,
ring A₂′ may correspond to ring A₂, and may be identical to or different from ring A₂, and
X₁, X₃, Y₁ to Y₃, ring A₁, and ring A₂ may each be the same as described herein.

In the condensed cyclic compound according to one or more embodiments,
n1 may be 0, and n2 may be 1, and
one selected from among Conditions 2A to 2C may be satisfied:
2A) m1 is 1, and m2 is 0;
2B) m1 is 2, and m2 is 0; and
2C) m1 is 1, and m2 is 1.

The condensed cyclic compound according to one or more embodiments may satisfy Condition 2A, and may be represented by Formula 2-A:

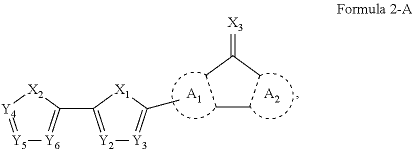

wherein, in Formula 2-A,
X₁ to X₃, Y₂ to Y₆, ring A₁, and ring A₂ may each be the same as described herein.

The condensed cyclic compound according to one or more embodiments may satisfy Condition 2B, and may be represented by Formula 2-B:

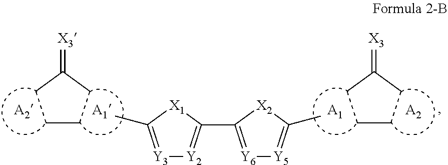

wherein, in Formula 2-B,
X₃′ may correspond to X₃, and may be identical to or different from X₃,
ring A₁′ may correspond to ring A₁, and may be identical to or different from ring A₁,
ring A₂′ may correspond to ring A₂, and may be identical to or different from ring A₂, and
X₁ to X₃, Y₂, Y₃, Y₅, Y₆, ring A₁, and ring A₂ may each be the same as described herein.

The condensed cyclic compound according to one or more embodiments may satisfy Condition 2C, and may be represented by Formula 2-C:

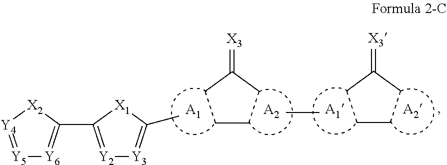

wherein, in Formula 2-C,
X₃′ may correspond to X₃, and may be identical to or different from X₃,
ring A₁′ may correspond to ring A₁, and may be identical to or different from ring A₁,
ring A₂′ may correspond to ring A₂, and may be identical to or different from ring A₂, and
X₁ to X₃, Y₂ to Y₆, ring A₁, and ring A₂ may each be the same as described herein.

In the condensed cyclic compound according to one or more embodiments,
n1 may be 2, and n2 may be 0, and
one selected from among Conditions 3A to 3C may be satisfied:
3A) m1 is 0, and m2 is 1;
3B) m1 is 0, and m2 is 2; and
3C) m1 is 1, and m2 is 1.

The condensed cyclic compound according to one or more embodiments may satisfy Condition 3A, and may be represented by Formula 3-A:

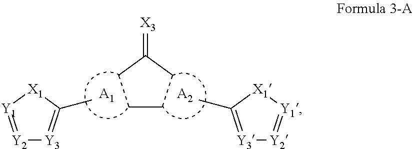

wherein, in Formula 3-A,
X₁′ may correspond to X₁, and may be identical to or different from X₁,
Y₁′ to Y₃′ may respectively correspond to Y₁ to Y₃, and may respectively be identical to or different from Y₁ to Y₃, and
X₁, X₃, Y₁ to Y₃, ring A₁, and ring A₂ may each be the same as described herein.

The condensed cyclic compound according to one or more embodiments may satisfy Condition 3B, and may be represented by Formula 3-B:

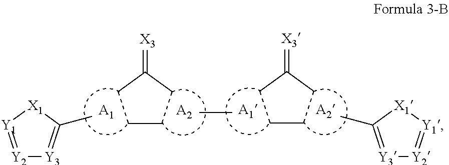

wherein, in Formula 3-B,
X₁′ may correspond to X₁, and may be identical to or different from X₁,
X₃′ may correspond to X₃, and may be identical to or different from X₃,
Y₁′ to Y₃′ may respectively correspond to Y₁ to Y₃, and may respectively be identical to or different from Y₁ to Y₃,
ring A₁′ may correspond to ring A₁, and may be identical to or different from ring A₁,
ring A₂′ may correspond to ring A₂, and may be identical to or different from ring A₂, and
X₁, X₃, Y₁ to Y₃, ring A₁, and ring A₂ may each be the same as described herein.

The condensed cyclic compound according to one or more embodiments may satisfy Condition 3C, and may be represented by Formula 3-C:

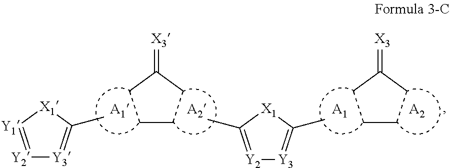

wherein, in Formula 3-C,
X₁′ may correspond to X₁, and may be identical to or different from X₁,
X₃′ may correspond to X₃, and may be identical to or different from X₃,
Y₁′ to Y₃′ may respectively correspond to Y₁ to Y₃ of Formula 1-1, and may respectively be identical to or different from Y₁ to Y₃,
ring A₁′ may correspond to ring A₁, and may be identical to or different from ring A₁,
ring A₂′ may correspond to ring A₂, and may be identical to or different from ring A₂, and
X₁, X₃, Y₁ to Y₃, ring A₁, and ring A₂ may each be the same as described herein.

In the condensed cyclic compound according to one or more embodiments,
n1 may be 0, and n2 may be 2, and
one selected from among Conditions 4A to 4E may be satisfied:
4A) m1 is 1, and m2 is 0;
4B) m1 is 2, and m2 is 0;
4C) m1 is 0, and m2 is 1;
4D) m1 is 0, and m2 is 2; and
4E) m1 is 1, and m2 is 1.

The condensed cyclic compound according to one or more embodiments may satisfy Condition 4A, and may be represented by Formula 4-A:

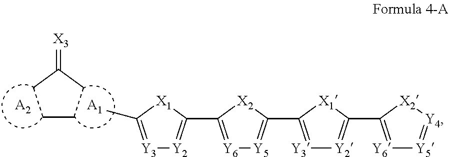

wherein, in Formula 4-A,
X₁′ and X₂′ may respectively correspond to X₁ and X₂, and may respectively be identical to or different from X₁ and X₂,
Y₂′, Y₃′, Y₅′, and Y₆′ may respectively correspond to Y₂, Y₃, Y₅, and Y₆, and may respectively be identical to or different from Y₂, Y₃, Y₅, and Y₆,
Y₃′ and Y₅ may optionally be bonded to each other to form: a C₃-C₆₀ carbocyclic group unsubstituted or substituted with at least one R₁₀ₐ; a C₁-C₆₀ heterocyclic group unsubstituted or substituted with at least one R₁₀ₐ; or a π electron-rich C₁-C₆₀ group unsubstituted or substituted with at least one R₁₀ₐ, and
X₁ to X₃, Y₂ to Y₆, ring A₁, ring A₂, and R₁₀ₐ may each be the same as described herein.

The condensed cyclic compound according to one or more embodiments may satisfy Condition 4B, and may be represented by Formula 4-B:

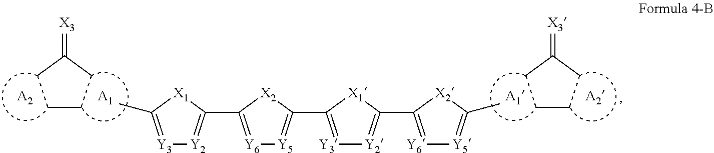

wherein, in Formula 4-B,
X₁′ and X₂′ may respectively correspond to X₁ and X₂, and may respectively be identical to or different from X₁ and X₂,
Y₂′, Y₃′, Y₅′, and Y₆′ may respectively correspond to Y₂, Y₃, Y₅, and Y₆, and may respectively be identical to or different from Y₂, Y₃, Y₅, and Y₆,
ring A₁′ may correspond to ring A₁, and may be identical to or different from ring A₁,
ring A₂′ may correspond to ring A₂, and may be identical to or different from ring A₂,
Y₃′ and Y₅ may optionally be bonded to each other to form: a C₃-C₆₀ carbocyclic group unsubstituted or substituted with at least one R₁₀ₐ; a C₁-C₆₀ heterocyclic group unsubstituted or substituted with at least one R₁₀ₐ; or a π electron-rich C₁-C₆₀ group unsubstituted or substituted with at least one R₁₀ₐ, and
X₁ to X₃, Y₂, Y₃, Y₅, Y₆, ring A₁, ring A₂, and R₁₀ₐ may each be the same as described herein.

The condensed cyclic compound according to one or more embodiments may satisfy Condition 4C, and may be represented by Formula 4-C:

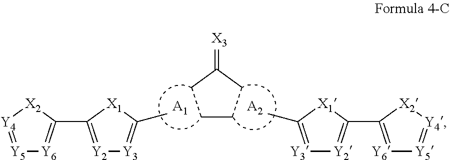

wherein, in Formula 4-C,
X₁′ and X₂′ may respectively correspond to X₁ and X₂, and may respectively be identical to or different from X₁ and X₂,
Y₂′ to Y₆′ may respectively correspond to Y₂ to Y₆, and may respectively be identical to or different from Y₂ to Y₆,
X₁, X₃, Y₂ to Y₆, ring A₁, and ring A₂ may each be the same as described herein.

The condensed cyclic compound according to one or more embodiments may satisfy Condition 4D, and may be represented by Formula 4-D:

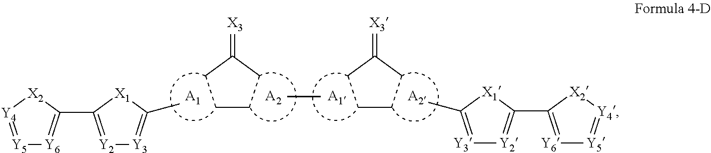

wherein, in Formula 4-D,
X₁′ to X₃′ may respectively correspond to X₁ to X₃, and may respectively be identical to or different from X₁ to X₃,
Y₂′ to Y₆′ may respectively correspond to Y₂ to Y₆, and may respectively be identical to or different from Y₂ to Y₆,
ring A₁′ may correspond to ring A₁, and may be identical to or different from ring A₁,
ring A₂′ may correspond to ring A₂, and may be identical to or different from ring A₂, and
X₁ to X₃, Y₂ to Y₆, ring A₁, and ring A₂ may each be the same as described herein.

The condensed cyclic compound according to one or more embodiments may satisfy Condition 4E, and may be represented by Formula 4-E:

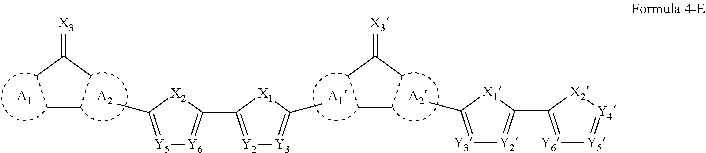

wherein, in Formula 4-E,
X₁′ to X₃′ may respectively correspond to X₁ to X₃, and may respectively be identical to or different from X₁ to X₃,
Y₂′ to Y₆′ may respectively correspond to Y₂ to Y₆ of Formula 1-2, and may respectively be identical to or different from Y₂ to Y₆,
ring A₁′ may correspond to ring A₁, and may be identical to or different from ring A₁,
ring A₂′ may correspond to ring A₂, and may be identical to or different from ring A₂, and
X₁ to X₃, Y₂ to Y₆, ring A₁, and ring A₂ may each be the same as described herein.

In the condensed cyclic compound according to one or more embodiments,
n1 may be 1, and n2 may be 1, and
one selected from among Conditions 5A to 5E may be satisfied:
5A) m1 is 1, and m2 is 0;
5B) m1 is 2, and m2 is 0;
5C) m1 is 0, and m2 is 1;
5D) m1 is 0, and m2 is 2; and
5E) m1 is 1, and m2 is 1.

The condensed cyclic compound according to one or more embodiments may satisfy Condition 5A, and may be represented by Formula 5-A:

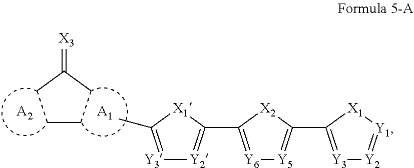

wherein, in Formula 5-A,
X₁′ may correspond to X₁, and may be identical to or different from X₁,
Y₂′ and Y₃′ may respectively correspond to Y₂ and Y₃, and may respectively be identical to or different from Y₂ and Y₃,
Y₃ and Y₅ may optionally be bonded to each other to form: a C₃-C₆₀ carbocyclic group unsubstituted or substituted with at least one R₁₀ₐ; a C₁-C₆₀ heterocyclic group unsubstituted or substituted with at least one R₁₀ₐ; or a π electron-rich C₁-C₆₀ group unsubstituted or substituted with at least one R₁₀ₐ, and
X₁ to X₃, Y₁ to Y₆, ring A₁, ring A₂, and R₁₀ₐ may each be the same as described herein.

The condensed cyclic compound according to one or more embodiments may satisfy Condition 5B, and may be represented by Formula 5-B:

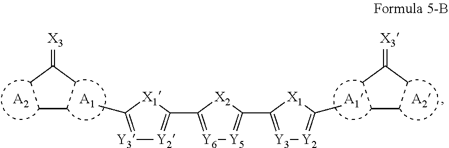

wherein, in Formula 5-B,
X₁′ and X₃′ may respectively correspond to X₁ and X₃, and may respectively be identical to or different from X₁ and X₃,
Y₂′ and Y₃′ may respectively correspond to Y₂ and Y₃, and may respectively be identical to or different from Y₂ and Y₃,
ring A₁′ may correspond to ring A₁, and may be identical to or different from ring A₁,
ring A₂′ may correspond to ring A₂, and may be identical to or different from ring A₂,
Y₃ and Y₅ may optionally be bonded to each other to form: a C₃-C₆₀ carbocyclic group unsubstituted or substituted with at least one R₁₀ₐ; a C₁-C₆₀ heterocyclic group unsubstituted or substituted with at least one R₁₀ₐ; or a π electron-rich C₁-C₆₀ group unsubstituted or substituted with at least one R₁₀ₐ, and
X₁ to X₃, Y₂, Y₃, Y₅, Y₆, ring A₁, ring A₂, and R₁₀ₐ may each be the same as described herein.

The condensed cyclic compound according to an embodiment may satisfy Condition 5C, and may be represented by Formula 5-C:

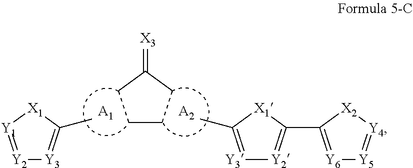

wherein, in Formula 5-C,
X₁′ may correspond to X₁, and may be identical to or different from X₁,
Y₂′ and Y₃′ may respectively correspond to Y₂ and Y₃, and may respectively be identical to or different from Y₂ and Y₃, and
X₁ to X₃, Y₁ to Y₆, ring A₁, and ring A₂ may each be the same as described herein.

The condensed cyclic compound according to one or more embodiments may satisfy Condition 5D, and may be represented by Formula 5-D:

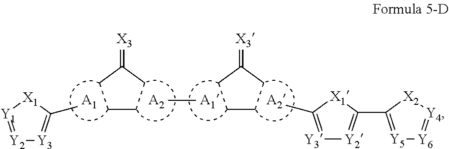

wherein, in Formula 5-D,
X₁′ and X₃′ may respectively correspond to X₁ and X₃, and may respectively be identical to or different from X₁ and X₃,
Y₂′ and Y₃′ may respectively correspond to Y₂ and Y₃, and may respectively be identical to or different from Y₂ and Y₃,
ring A₁′ may correspond to ring A₁, and may be identical to or different from ring A₁,
ring A₂′ may correspond to ring A₂, and may be identical to or different from ring A₂, and
X₁ to X₃, Y₁ to Y₆, ring A₁, and ring A₂ may each be the same as described herein.

The condensed cyclic compound according to one or more embodiments may satisfy Condition 5E, and may be represented by Formula 5-E-1 or 5-E-2:

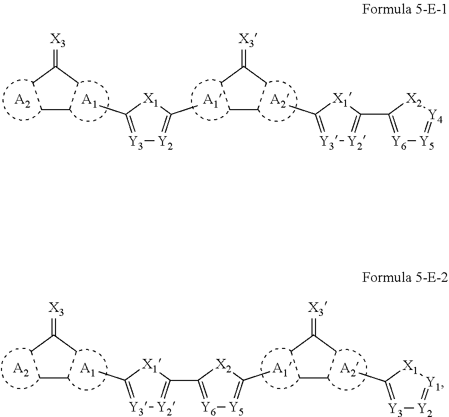

wherein, in Formulae 5-E-1 and 5-E-2,
X₁′ and X₃′ may respectively correspond to X₁ and X₃, and may respectively be identical to or different from X₁ and X₃,
Y₂′ and Y₃′ may respectively correspond to Y₂ and Y₃, and may respectively be identical to or different from Y₂ and Y₃,
ring A₁′ may correspond to ring A₁, and may be identical to or different from ring A₁,
ring A₂′ may correspond to ring A₂, and may be identical to or different from ring A₂, and
X₁ to X₃, Y₁ to Y₆, ring A₁, and ring A₂ may each be the same as described herein.

In the condensed cyclic compound according to one or more embodiments, X₁ and X₂ may each independently be S or Se.

In the condensed cyclic compound according to one or more embodiments,
when the sum of m1 and m2 is 1, X₃ may be C(CN)(CN), C(CN)H, or N(CN), and
when the sum of m1 and m2 is 2 or more, X₃(s) included in the second-first moiety or the second-second moiety may each independently be C(CN)(CN), C(CN)H, or N(CN).

In the condensed cyclic compound according to one or more embodiments,
ring A₁ and ring A₂ may each independently be a 5-membered cyclic group, a 5-membered heterocyclic group, a 6-membered cyclic group, a 6-membered heterocyclic group, a polycyclic C₆-C₃₀ cyclic group including a 5-membered cyclic group, a polycyclic C₇-C₃₀ cyclic group including a 6-membered cyclic group, a polycyclic C₅-C₃₀ heterocyclic group including a 5-membered cyclic group, a polycyclic C₆-C₃₀ heterocyclic group including a 6-membered cyclic group, a polycyclic C₁-C₃₀ heterocyclic group including a 5-membered heterocyclic group, or a polycyclic C₁-C₃₀ heterocyclic group including a 6-membered heterocyclic group.

In the condensed cyclic compound according to one or more embodiments, a moiety represented by

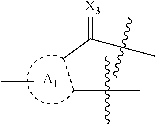

in Formula 2-1 or 2-2 may include a group represented by one selected from among Formulae 4-1-1 to 4-1-4:

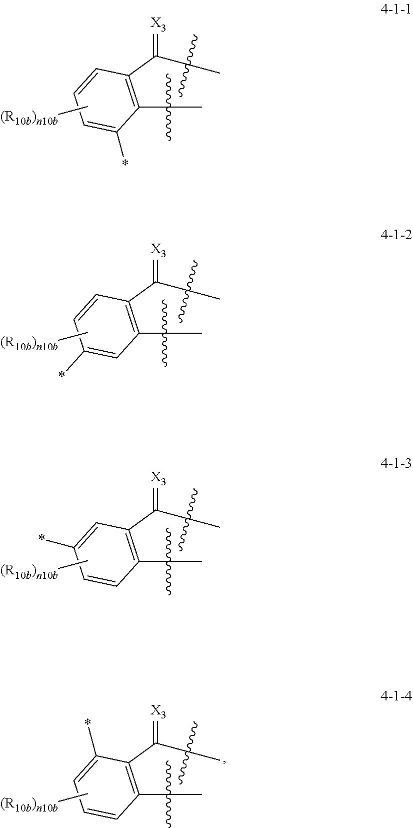

wherein, in Formulae 4-1-1 to 4-1-4,
n10b may be an integer from 0 to 3,
[symbol] may indicate a linking site to ring A₂,
* may indicate a linking site to a neighboring atom, and
R_{10b} and X₃ may each be the same as described herein.

In the condensed cyclic compound according to one or more embodiments,
a moiety represented by

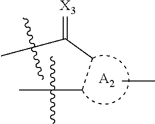

in Formula 2-2 may include a group represented by one selected from among Formulae 4-2-1 to 4-2-4:

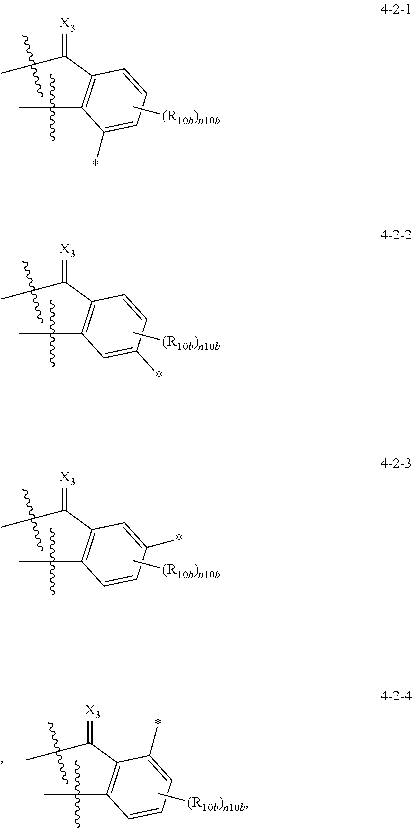

wherein, in Formulae 4-2-1 to 4-2-4,
n10b may be an integer from 0 to 3,
[symbol] may indicate a linking site to ring A₁,
* may indicate a linking site to a neighboring atom, and
R_{10b} and X₃ may each be the same as described herein.

The condensed cyclic compound according to one or more embodiments may satisfy one selected from among Conditions i) to iv):
i) the condensed cyclic compound includes a group represented by Formula 4-1-2 or a group represented by Formula 4-2-2, and absorbs green light;
ii) the condensed cyclic compound includes a group represented by Formula 4-1-2 or a group represented by Formula 4-2-2, and absorbs red light;
iii) the condensed cyclic compound includes at least one selected from among a group represented by Formula 4-1-1, a group represented by Formula 4-1-3, a group represented by Formula 4-2-1, and a group represented by Formula 4-2-3, and absorbs green light; and
iv) the condensed cyclic compound includes at least one selected from among a group represented by Formula 4-1-1, a group represented by Formula 4-1-3, a group represented by Formula 4-2-1, and a group represented by Formula 4-2-3, and absorbs red light.

In the condensed cyclic compound according to one or more embodiments,
the π electron-rich C₁-C₆₀ group may include at least one selected from among *—C(Q₁)(Q₂)-*, a π electron-rich C₃-C₆₀ cyclic group, and groups represented by Formulae 3-1 to 3-3:

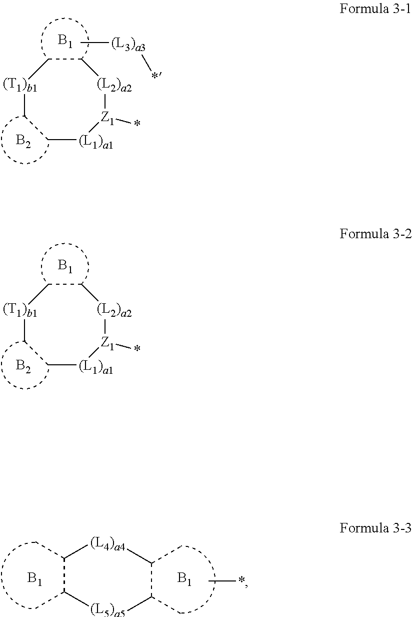

wherein, in Formulae 3-1 to 3-3,
Z₁ may be C(R₉), N, or P,
ring B₁ and ring B₂ may each independently include: a C₃-C₆₀ carbocyclic group unsubstituted or substituted with at least one R₁₀ₐ; or a C₁-C₆₀ heterocyclic group unsubstituted or substituted with at least one R₁₀ₐ,
L₁ to L₅ may each independently be *—O—*′, *—S—*′, *—C(Q₁)(Q₂)-*′, *—C(Q₁)═*′, *═C(Q₁)-*′, *—Ci(Q₁)=C(Q₂)-*′, *—C≡C—*′, *—N(Q₁)-*′, *—P(Q₁)-*′, *—Si(Q₁)(Q₂)-*′, *—P(Q₁)(Q₂)-* or *—Ge(Q₁)(Q₂)-*′,
a1 to a5 may each independently be an integer from 0 to 5,
T₁ may be a single bond, *—O*′, *—S—*′, *—C(Q₁)(Q₂)-*′, *—C(Q₁)═*′, *═C(Q₁)-*′, *—C(Q₁)═C(Q₂)-*′, *—C≡C—*, *—N(Q₁)-*′, *—P(Q₁)-*′, *—Si(Q₁)(Q₂)-*′, *—P(Q₁)(Q₂)-*′, or *—Ge(Q₁)(Q₂)-*′,
b1 may be an integer from 0 to 5,
when a1 is 0, *-(L₁)ₐ₁-*′ may be a single bond,
when a2 is 0, *-(L₂)ₐ₂-*′ may be a single bond,
when a3 is 0, *-(L₃)ₐ₃-*′ may be a single bond,
when a4 is 0, *-(L₄)ₐ₄-*′ may be a single bond,
when a5 is 0, *-(L₅)ₐ₅-*′ may be a single bond,
when b1 is 0, ring B₁ and ring B₂ may not be directly linked to each other,
Q₁ and Q₂ may respectively correspond to Q₁₁ and Q₁₂, and
* and *′ may each indicate a linking site to the first repeating unit.

In the condensed cyclic compound according to one or more embodiments,
in Formulae 3-1 and 3-2,
ring B₁ and ring B₂ may each independently be a 5-membered cyclic group, a 5-membered heterocyclic group, a 6-membered cyclic group, a 6-membered heterocyclic group, a polycyclic C₆-C₃₀ cyclic group including a 5-membered cyclic group, a polycyclic C₇-C₃₀ cyclic group including a 6-membered cyclic group, a polycyclic C₅-C₃₀ heterocyclic group including a 5-membered cyclic group, a polycyclic C₆-C₃₀ heterocyclic group including a 6-membered cyclic group, a polycyclic C₁-C₃₀ heterocyclic group including a 5-membered heterocyclic group, or a polycyclic C₁-C₃₀ heterocyclic group including a 6-membered heterocyclic group.

In the condensed cyclic compound according to one or more embodiments,
the π electron-rich C₁-C₆₀ group may include a group represented by one selected from among Formulae 3-1-1 to 3-1-6:

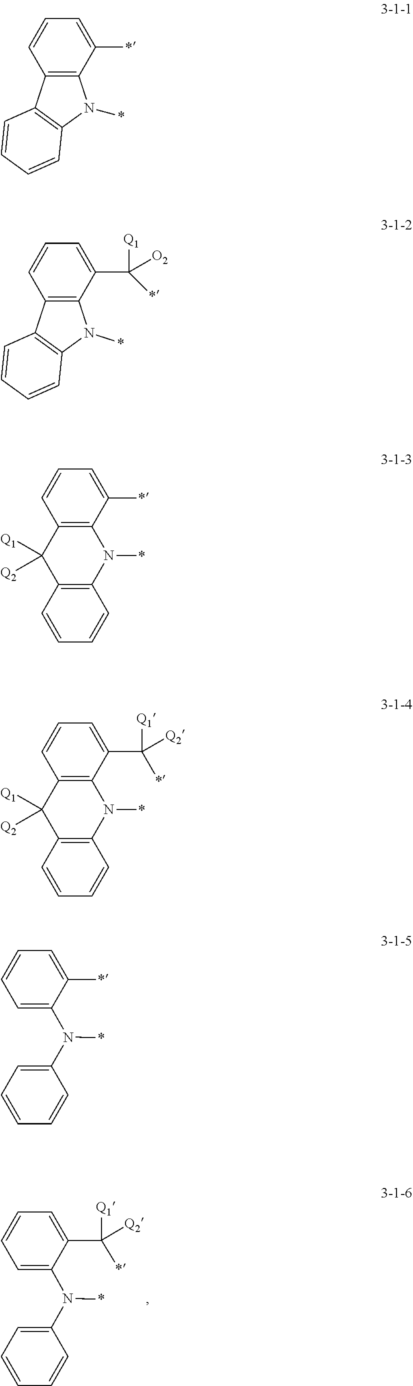

wherein, in Formulae 3-1-1 to 3-1-6,
Q₁′ and Q₂′ may respectively correspond to Q₁ and Q₂, and may respectively be identical to or different from Q₁ and Q₂,
* and *′ may each indicate a linking site to the first repeating unit, and
Q₁ and Q₂ may each be the same as described herein.

In the condensed cyclic compound according to one or more embodiments,
the π electron-rich C₁-C₆₀ group may include a group represented by one selected from among Formulae 3-3-1 to 3-3-17:

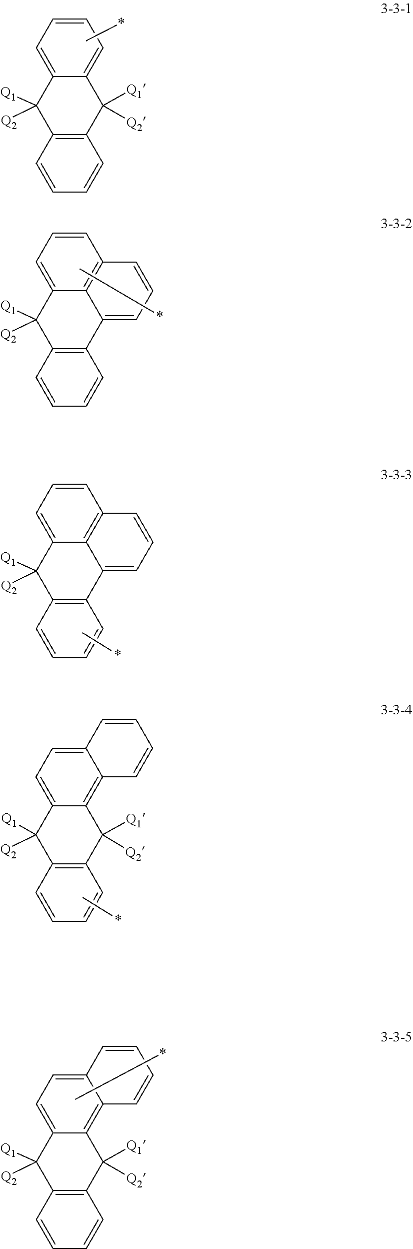

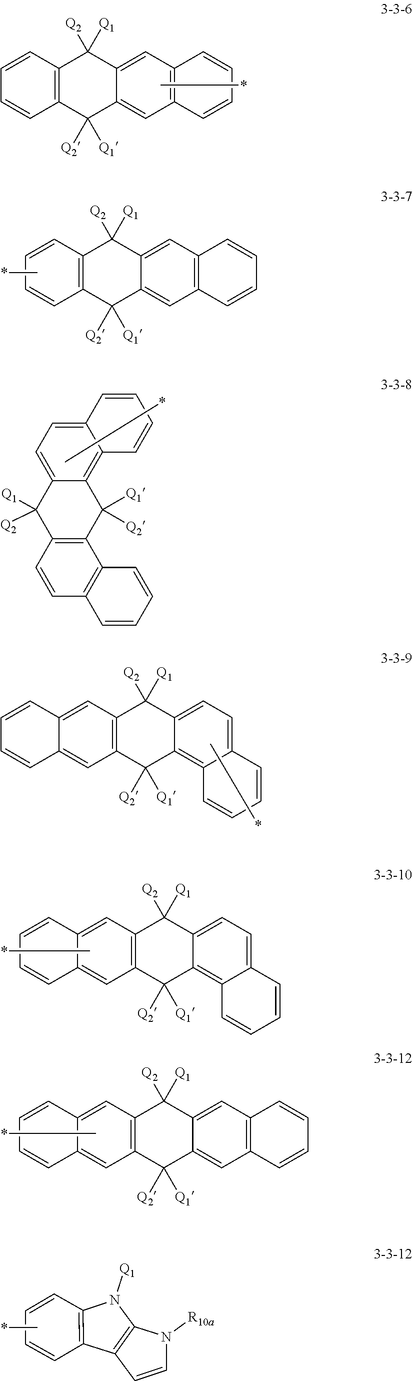

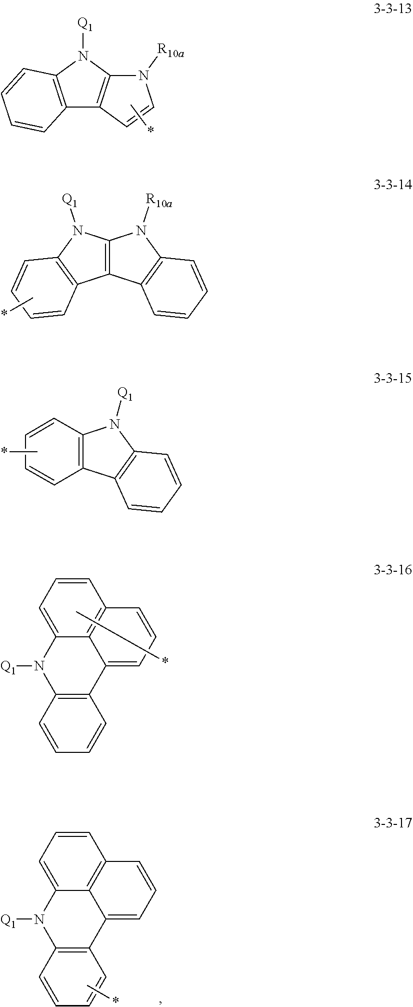

wherein, in Formulae 3-3-1 to 3-3-17,
Q₁′ and Q₂′ may respectively correspond to Q₁ and Q₂, and may respectively be identical to or different from Q₁ and Q₂,
* may indicate a linking site to the first repeating unit, and
Q₁, Q₂, and R₁₀ₐ may each be the same as described herein.

In the condensed cyclic compound according to one or more embodiments, the π electron-rich C₁-C₆₀ group may not be a halogen.

The condensed cyclic compound according to one or more embodiments may further include at least one linker moiety,
wherein the linker moiety may bridge between the first repeating unit and the second repeating unit;
the linker moiety may bridge between one first repeating unit and another first repeating unit; or
the linker moiety may bridge between one second repeating unit and another second repeating unit, and
the linker moiety may include *—C(Q₃)═C(Q₄)-*′, *—C≡C—*′, a C₅-C₆₀ carbocyclic group unsubstituted or substituted with at least one R_{10b}, or a C₁-C₆₀ heterocyclic group unsubstituted or substituted with at least one R_{10b}.
Q₃ and Q₄ may respectively be the same as described with respect to Q₄₁ and Q₄₂, and R_{10b} may be the same as described herein.

In the condensed cyclic compound according to one or more embodiments,
the linker moiety may include one selected from among groups represented by Formulae 5-1 to 5-3:

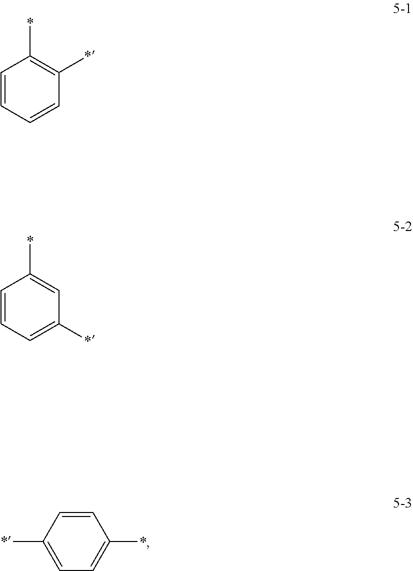

wherein, in Formulae 5-1 to 5-3,
* and *′ may each indicate a linking site to a neighboring repeating unit or linker.

The condensed cyclic compound according to one or more embodiments may include
at least one electron-accepting moiety and at least one spacer moiety.

The electron-accepting moiety may include a 9-methylenefluorene moiety and at least one cyano group linked to the 9-methylenefluorene moiety,
the spacer moiety may include a thiophene moiety and/or a selenophene moiety,
the spacer moiety may further include at least one π electron-rich C₁-C₆₀ group, and
the π electron-rich C₁-C₆₀ group may not be directly linked to the electron-accepting moiety.

A lowest unoccupied molecular orbital (LUMO) energy level of the condensed cyclic compound according to one or more embodiments may be less than −2.5 eV, and a highest occupied molecular orbital (HOMO) energy level thereof may be less than −4.5 eV.

The condensed cyclic compound according to one or more embodiments may be to absorb green light or red light.

The condensed cyclic compound according to one or more embodiments may be one selected from among Compounds 1 to 48 and R01 to R07:

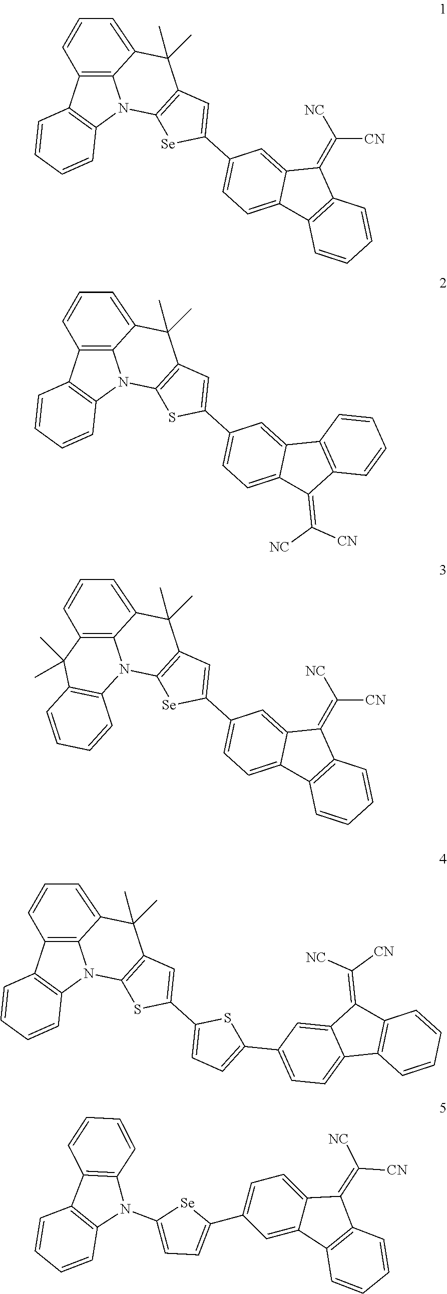 [SMILES: CC1(C)c2cc(-c3ccc4c(c3)C(=C(C#N)C#N)c3ccccc3-4)[se]c2-n2c3ccccc3c3cccc1c32 ; CC1(C)c2cc(-c3ccc4c(c3)-c3ccccc3C4=C(C#N)C#N)sc2-n2c3ccccc3c3cccc1c32 ; CC1(C)c2ccccc2N2c3[se]c(-c4ccc5c(c4)C(=C(C#N)C#N)c4ccccc4-5)cc3C(C)(C)c3cccc1c32 ; CC1(C)c2cc(-c3ccc(-c4ccc5c(c4)C(=C(C#N)C#N)c4ccccc4-5)s3)sc2-n2c3ccccc3c3cccc1c32 ; N#CC(C#N)=C1c2ccccc2-c2cc(-c3ccc(-n4c5ccccc5c5ccccc54)[se]3)ccc21]

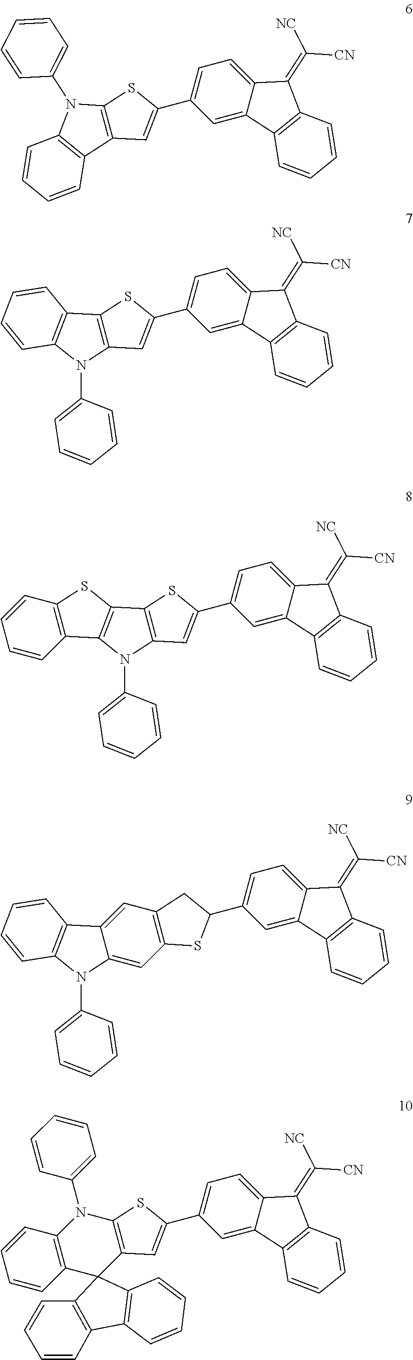 [SMILES: N#CC(C#N)=C1c2ccccc2-c2cc(-c3cc4c5ccccc5n(-c5ccccc5)c4s3)ccc21 ; N#CC(C#N)=C1c2ccccc2-c2cc(-c3cc4c(s3)c3ccccc3n4-c3ccccc3)ccc21 ; N#CC(C#N)=C1c2ccccc2-c2cc(-c3cc4c(s3)c3sc5ccccc5c3n4-c3ccccc3)ccc21 ; N#CC(C#N)=C1c2ccccc2-c2cc(C3Cc4cc5c6ccccc6n(-c6ccccc6)c5cc4S3)ccc21 ; N#CC(C#N)=C1c2ccccc2-c2cc(-c3cc4c(s3)N(c3ccccc3)c3ccccc3C43c4ccccc4-c4ccccc43)ccc21]

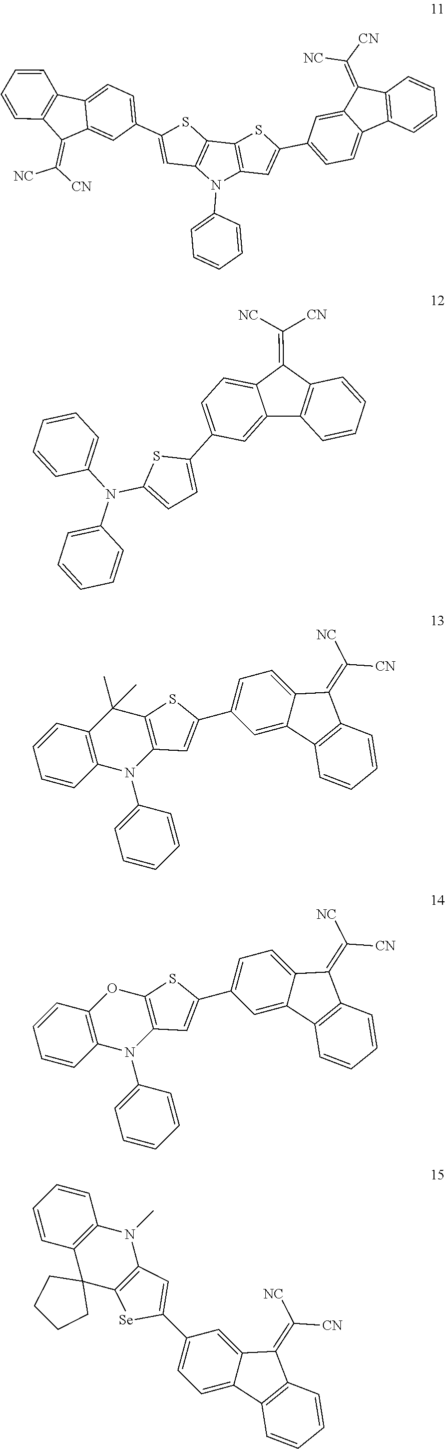 [SMILES: N#CC(C#N)=C1c2ccccc2-c2ccc(-c3cc4c(s3)c3sc(-c5ccc6c(c5)C(=C(C#N)C#N)c5ccccc5-6)cc3n4-c3ccccc3)cc21 ; N#CC(C#N)=C1c2ccccc2-c2cc(-c3ccc(N(c4ccccc4)c4ccccc4)s3)ccc21 ; CC1(C)c2ccccc2N(c2ccccc2)c2cc(-c3ccc4c(c3)-c3ccccc3C4=C(C#N)C#N)sc21 ; N#CC(C#N)=C1c2ccccc2-c2cc(-c3cc4c(s3)Oc3ccccc3N4c3ccccc3)ccc21 ; CN1c2ccccc2C2(CCCC2)c2[se]c(-c3ccc4c(c3)C(=C(C#N)C#N)c3ccccc3-4)cc21]

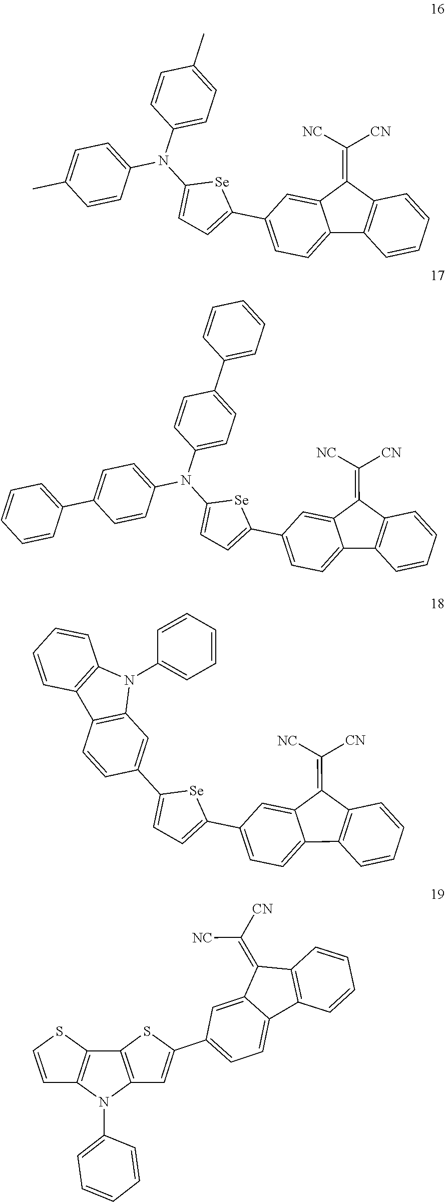 [SMILES: Cc1ccc(N(c2ccc(C)cc2)c2ccc(-c3ccc4c(c3)C(=C(C#N)C#N)c3ccccc3-4)[se]2)cc1 ; N#CC(C#N)=C1c2ccccc2-c2ccc(-c3ccc(N(c4ccc(-c5ccccc5)cc4)c4ccc(-c5ccccc5)cc4)[se]3)cc21 ; N#CC(C#N)=C1c2ccccc2-c2ccc(-c3ccc(-c4ccc5c6ccccc6n(-c6ccccc6)c5c4)[se]3)cc21 ; N#CC(C#N)=C1c2ccccc2-c2ccc(-c3cc4c(s3)c3sccc3n4-c3ccccc3)cc21]

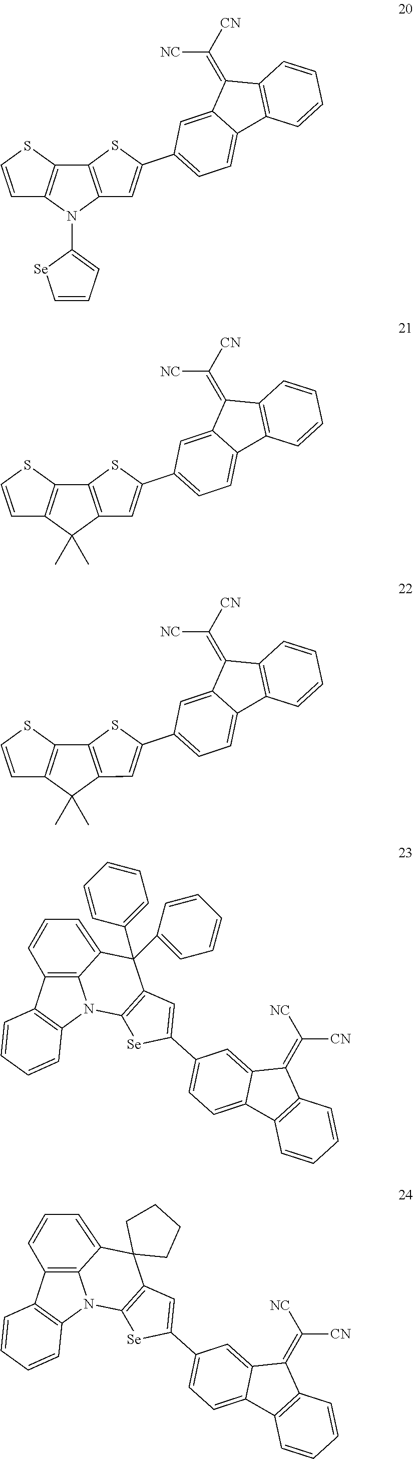

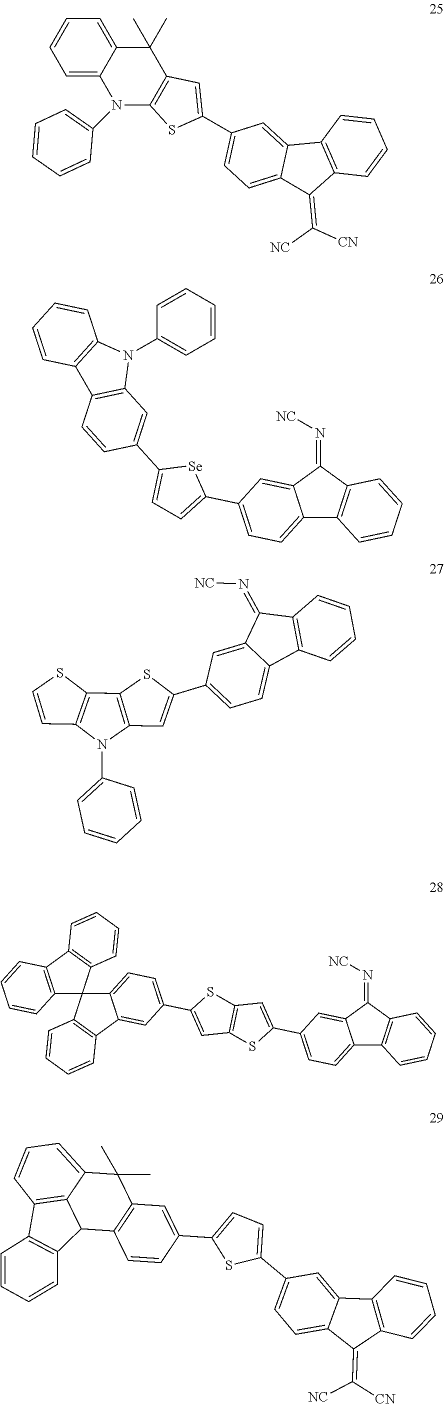 [SMILES: CC1(C)c2ccccc2N(c2ccccc2)c2sc(-c3ccc4c(c3)-c3ccccc3C4=C(C#N)C#N)cc21 ; N#C/N=C1/c2ccccc2-c2ccc(-c3ccc(-c4ccc5c6ccccc6n(-c6ccccc6)c5c4)[se]3)cc21 ; N#C/N=C1/c2ccccc2-c2ccc(-c3cc4c(s3)c3sccc3n4-c3ccccc3)cc21 ; N#C/N=C1/c2ccccc2-c2ccc(-c3cc4sc(-c5ccc6c(c5)-c5ccccc5C65c6ccccc6-c6ccccc65)cc4s3)cc21 ; CC1(C)c2cc(-c3ccc(-c4ccc5c(c4)-c4ccccc4C5=C(C#N)C#N)s3)ccc2C2c3ccccc3-c3cccc1c32]

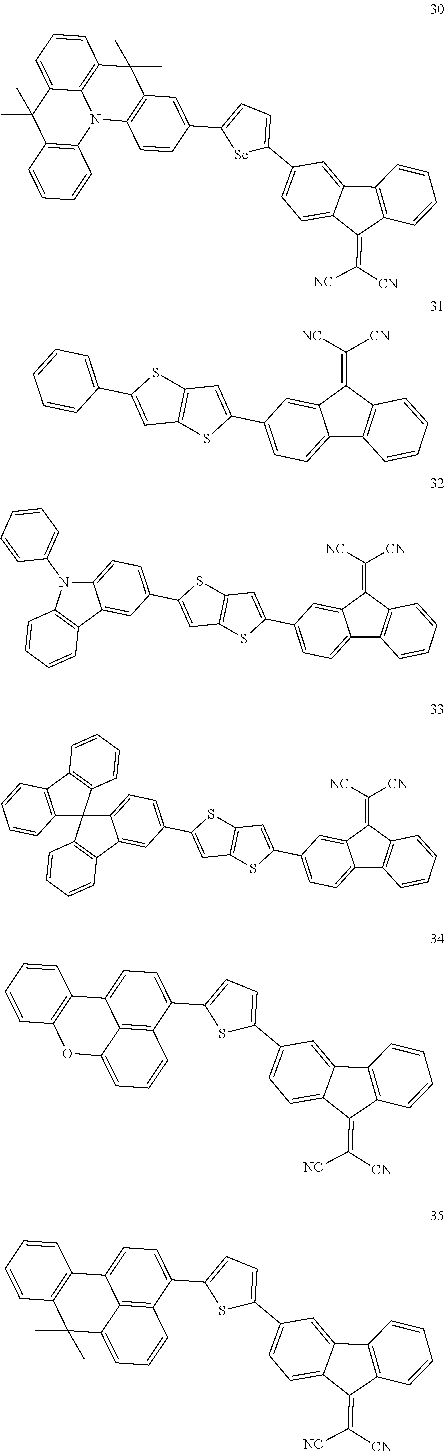 [SMILES: CC1(C)c2ccccc2N2c3ccc(-c4ccc(-c5ccc6c(c5)-c5ccccc5C6=C(C#N)C#N)[se]4)cc3C(C)(C)c3cccc1c32 ; N#CC(C#N)=C1c2ccccc2-c2ccc(-c3cc4sc(-c5ccccc5)cc4s3)cc21 ; N#CC(C#N)=C1c2ccccc2-c2ccc(-c3cc4sc(-c5ccc6c(c5)c5ccccc5n6-c5ccccc5)cc4s3)cc21 ; N#CC(C#N)=C1c2ccccc2-c2ccc(-c3cc4sc(-c5ccc6c(c5)-c5ccccc5C65c6ccccc6-c6ccccc65)cc4s3)cc21 ; N#CC(C#N)=C1c2ccccc2-c2cc(-c3ccc(-c4ccc5c6c(cccc46)Oc4ccccc4-5)s3)ccc21 ; CC1(C)c2ccccc2-c2ccc(-c3ccc(-c4ccc5c(c4)-c4ccccc4C5=C(C#N)C#N)s3)c3cccc1c23]

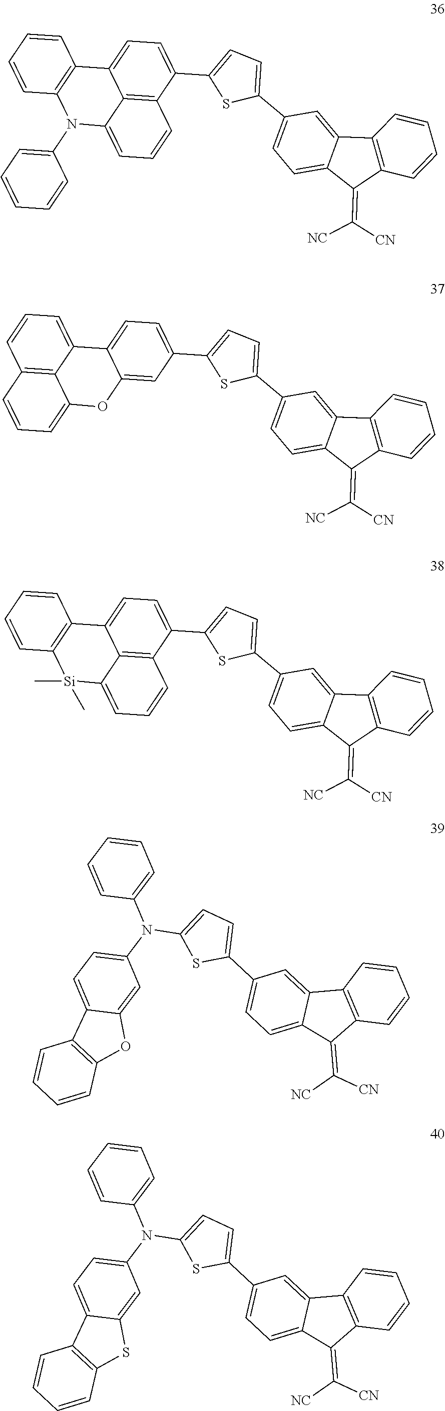 [SMILES: N#CC(C#N)=C1c2ccccc2-c2cc(-c3ccc(-c4ccc5c6c(cccc46)N(c4ccccc4)c4ccccc4-5)s3)ccc21 ; N#CC(C#N)=C1c2ccccc2-c2cc(-c3ccc(-c4ccc5c(c4)Oc4cccc6cccc-5c46)s3)ccc21 ; C[Si]1(C)c2ccccc2-c2ccc(-c3ccc(-c4ccc5c(c4)-c4ccccc4C5=C(C#N)C#N)s3)c3cccc1c23 ; N#CC(C#N)=C1c2ccccc2-c2cc(-c3ccc(N(c4ccccc4)c4ccc5c(c4)oc4ccccc45)s3)ccc21 ; N#CC(C#N)=C1c2ccccc2-c2cc(-c3ccc(N(c4ccccc4)c4ccc5c(c4)sc4ccccc45)s3)ccc21]

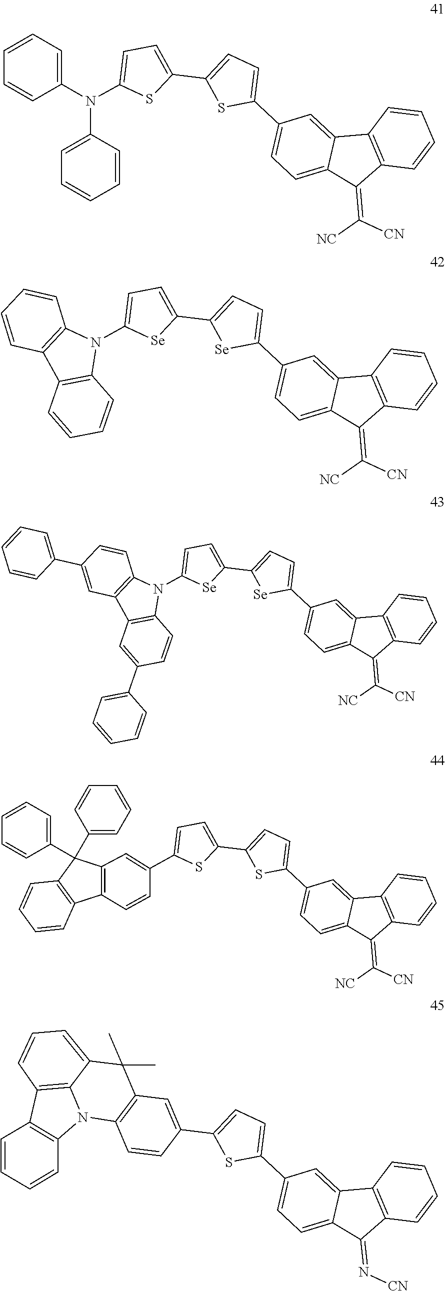

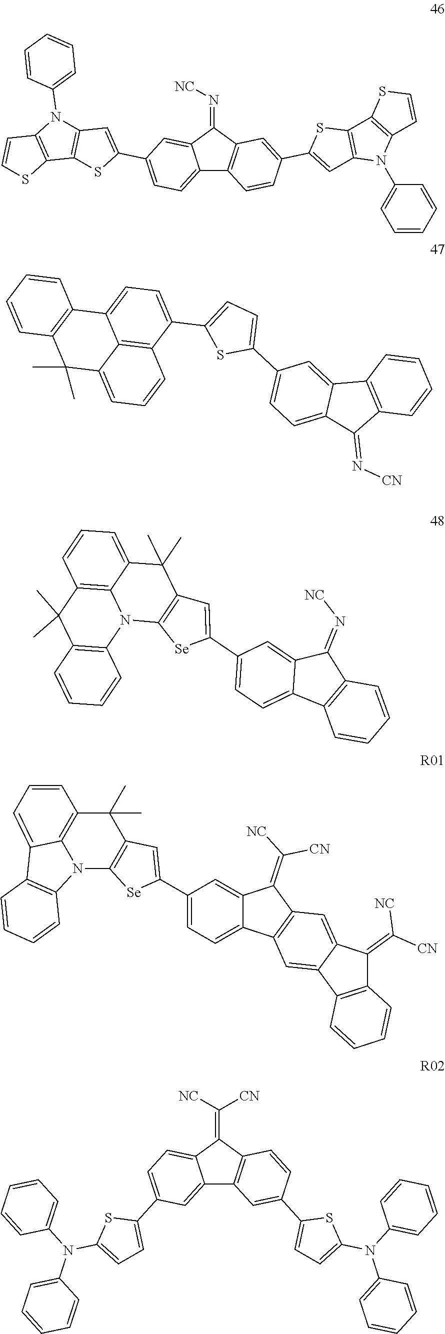 [SMILES: N#CN=C1c2cc(-c3cc4c(s3)c3sccc3n4-c3ccccc3)ccc2-c2ccc(-c3cc4c(s3)c3sccc3n4-c3ccccc3)cc21 ; CC1(C)c2ccccc2-c2ccc(-c3ccc(-c4ccc5c(c4)-c4ccccc4/C5=N\C#N)s3)c3cccc1c23 ; CC1(C)c2ccccc2N2c3[se]c(-c4ccc5c(c4)/C(=N\C#N)c4ccccc4-5)cc3C(C)(C)c3cccc1c32 ; CC1(C)c2cc(-c3ccc4c(c3)C(=C(C#N)C#N)c3cc5c(cc3-4)-c3ccccc3C5=C(C#N)C#N)[se]c2-n2c3ccccc3c3cccc1c32 ; N#CC(C#N)=C1c2ccc(-c3ccc(N(c4ccccc4)c4ccccc4)s3)cc2-c2cc(-c3ccc(N(c4ccccc4)c4ccccc4)s3)ccc21]

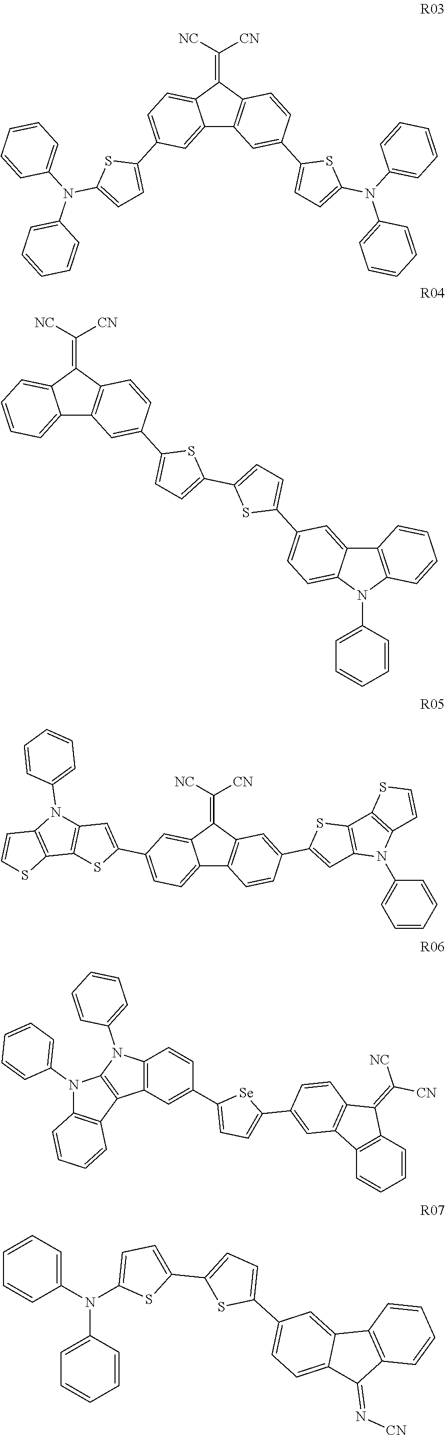 [SMILES: N#CC(C#N)=C1c2ccc(-c3ccc(N(c4ccccc4)c4ccccc4)s3)cc2-c2cc(-c3ccc(N(c4ccccc4)c4ccccc4)s3)ccc21 ; N#CC(C#N)=C1c2ccccc2-c2cc(-c3ccc(-c4ccc(-c5ccc6c(c5)c5ccccc5n6-c5ccccc5)s4)s3)ccc21 ; N#CC(C#N)=C1c2cc(-c3cc4c(s3)c3sccc3n4-c3ccccc3)ccc2-c2ccc(-c3cc4c(s3)c3sccc3n4-c3ccccc3)cc21 ; N#CC(C#N)=C1c2ccccc2-c2cc(-c3ccc(-c4ccc5c(c4)c4c6ccccc6n(-c6ccccc6)c4n5-c4ccccc4)[se]3)ccc21 ; N#C/N=C1\c2ccccc2-c2cc(-c3ccc(-c4ccc(N(c5ccccc5)c5ccccc5)s4)s3)ccc21]

In one or more embodiments, a light-receiving device may include: a first electrode; a second electrode facing the first electrode; an interlayer between the first electrode and the second electrode and including a light-receiving layer; and
one or more of the condensed cyclic compounds according to one or more embodiments.

In the light-receiving device according to one or more embodiments,
the first electrode may be an anode,
the second electrode may be a cathode, and
the interlayer between the first electrode and the second electrode and including the light-receiving layer may further include:
i) a hole transport region between the first electrode and the light-receiving layer and including a hole injection layer, a hole transport layer, an auxiliary layer, an electron-blocking layer, or any combination thereof; and
ii) an electron transport region between the light-receiving layer and the second electrode and including a hole-blocking layer, an electron transport layer, an electron injection layer, or any combination thereof.

In the light-receiving device according to one or more embodiments, the light-receiving layer may include the condensed cyclic compound of the present disclosure.

In the light-receiving device according to one or more embodiments, the light-receiving layer may be to absorb green light or red light.

The light-receiving device according to one or more embodiments may include: a first capping layer arranged outside the first electrode and including the condensed cyclic compound according to one or more embodiments; a second capping layer arranged outside the second electrode and including the condensed cyclic compound according to one or more embodiments; or the first capping layer and the second capping layer.

In the light-receiving device according to one or more embodiments, the interlayer may further include a photoelectric conversion material.

In the light-receiving device according to one or more embodiments, the photoelectric conversion material may include a compound represented by Formula 2:

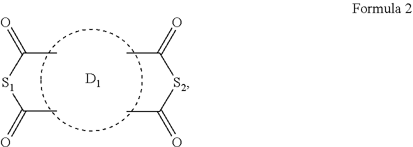

wherein, in Formula 2,
ring D₁ may include a C₃-C₆₀ carbocyclic group unsubstituted or substituted with at least one R₁₀ₐ, a C₁-C₆₀ heterocyclic group unsubstituted or substituted with at least one R₁₀ₐ, or a biphenyl group unsubstituted or substituted with at least one R₁₀ₐ,
S₁ and S₂ may each independently be C(R₁₁)(R₁₂), C(R₁₁)(H), N(R₁₁), N(H), or O,
R₁₁, R₁₂, and R₁₀ₐ may each independently be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a π electron-rich C₁-C₆₀ group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, or a π electron-rich C₁-C₆₀ group, each unsubstituted or substituted with deuterium, —F, a cyano group, a C₁-C₆₀ alkyl group, a C₁-C₆₀ alkoxy group, a phenyl group, a biphenyl group, or any combination thereof, and
two neighboring substituents selected from among Q₁₁ to Q₁₃, Q₂₁ to Q₂₃, and Q₃₁ to Q₃₃ may optionally be bonded to each other to form: a C₃-C₆₀ carbocyclic group unsubstituted or substituted with at least one R₁₀ₐ; a C₁-C₆₀ heterocyclic group unsubstituted or substituted with at least one R₁₀ₐ; or a π electron-rich C₁-C₆₀ group unsubstituted or substituted with at least one R₁₀ₐ.

In the light-receiving device according to one or more embodiments, the photoelectric conversion material may include a compound represented by one of Formulae 2-1 to 2-6:

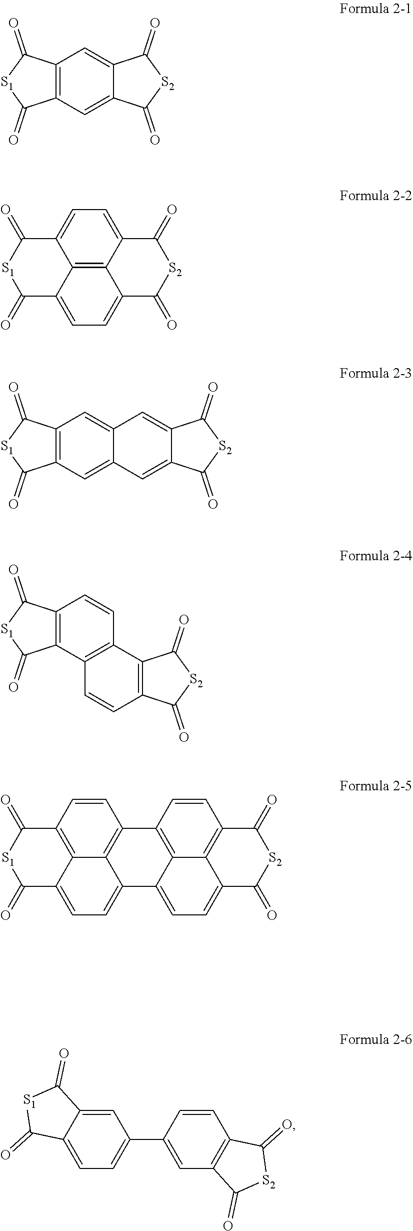

wherein, in Formulae 2-1 to 2-6,
S₁ and S₂ may respectively be the same as described with respect to S₁ and S₂ of Formula 2.

In the light-receiving device according to one or more embodiments, the photoelectric conversion material may be one selected from Compounds N1 to N43:

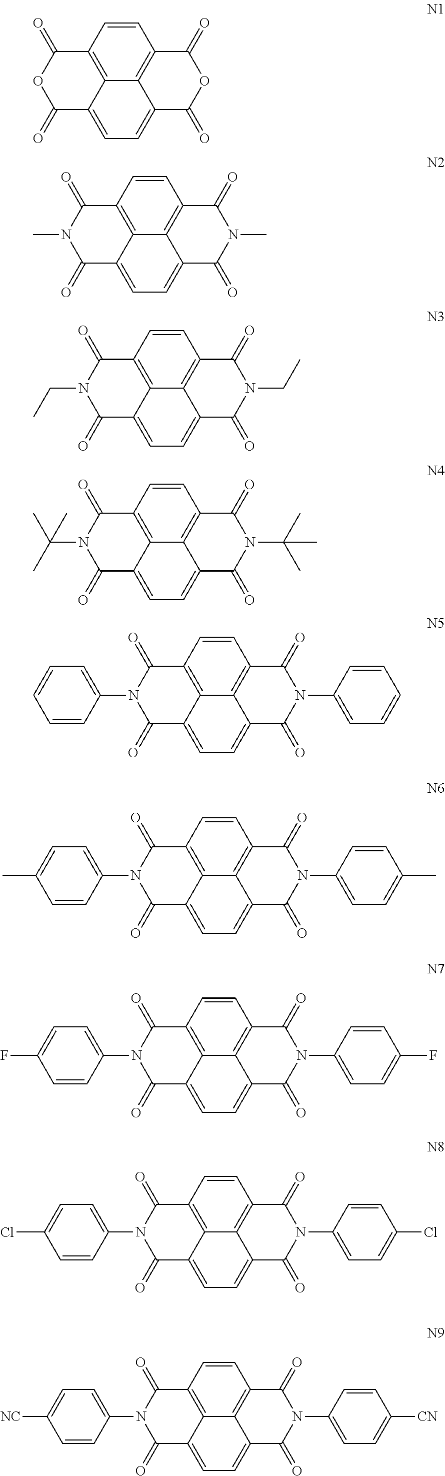

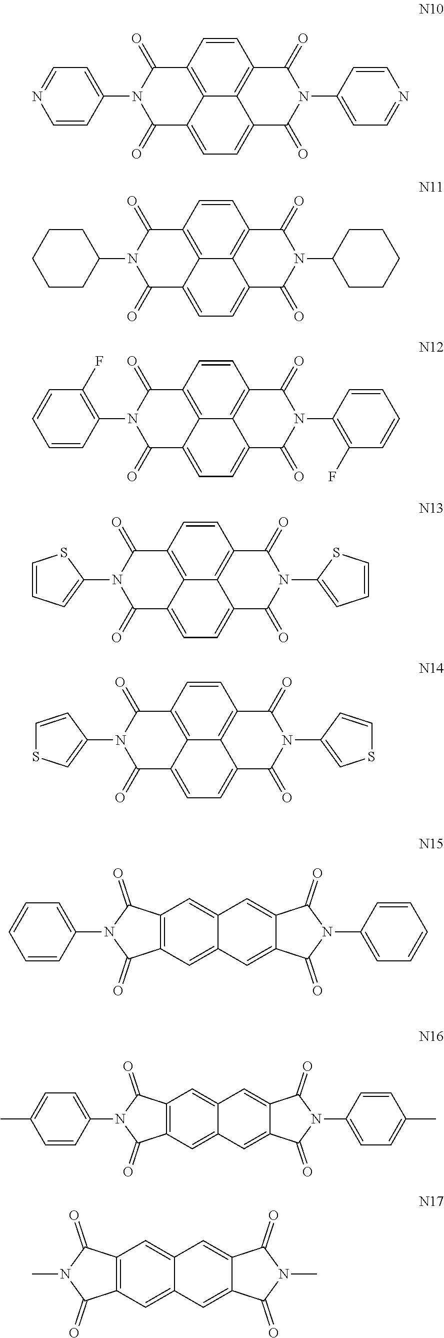

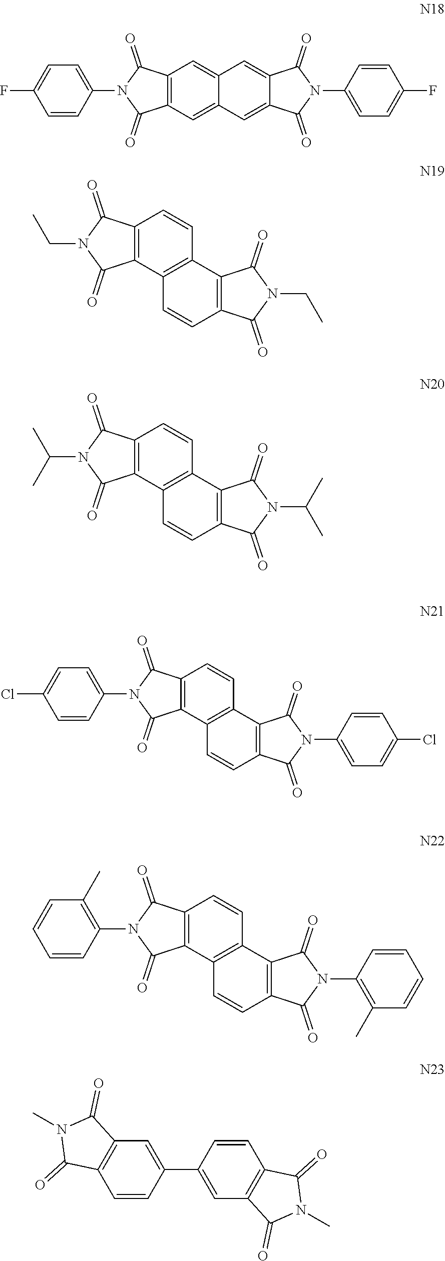

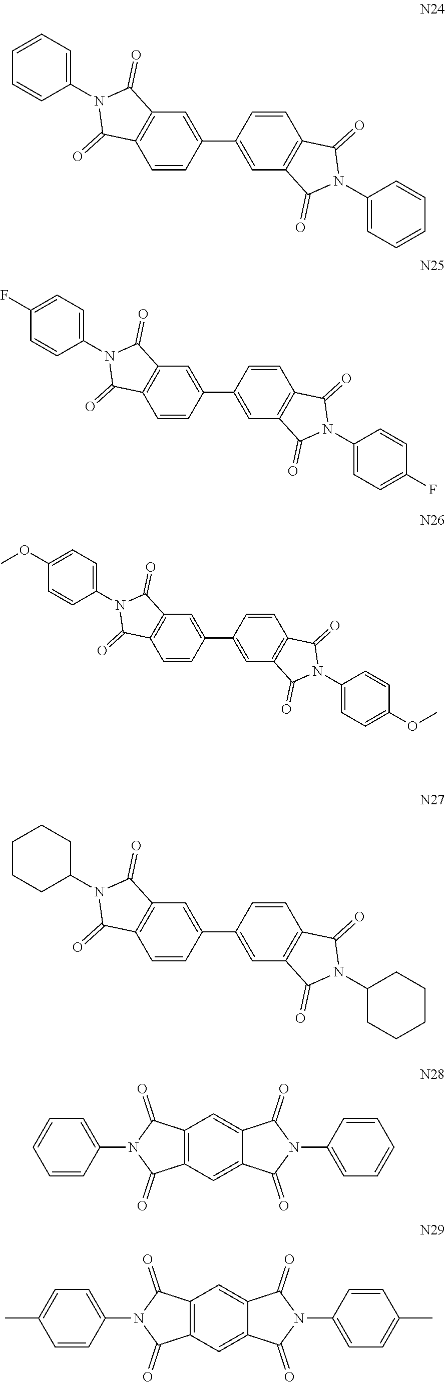

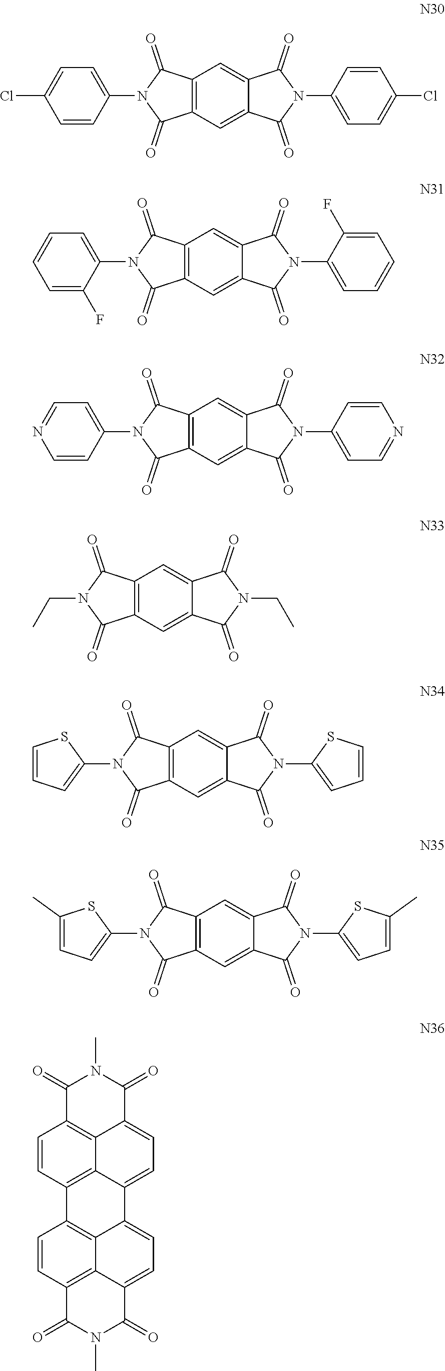

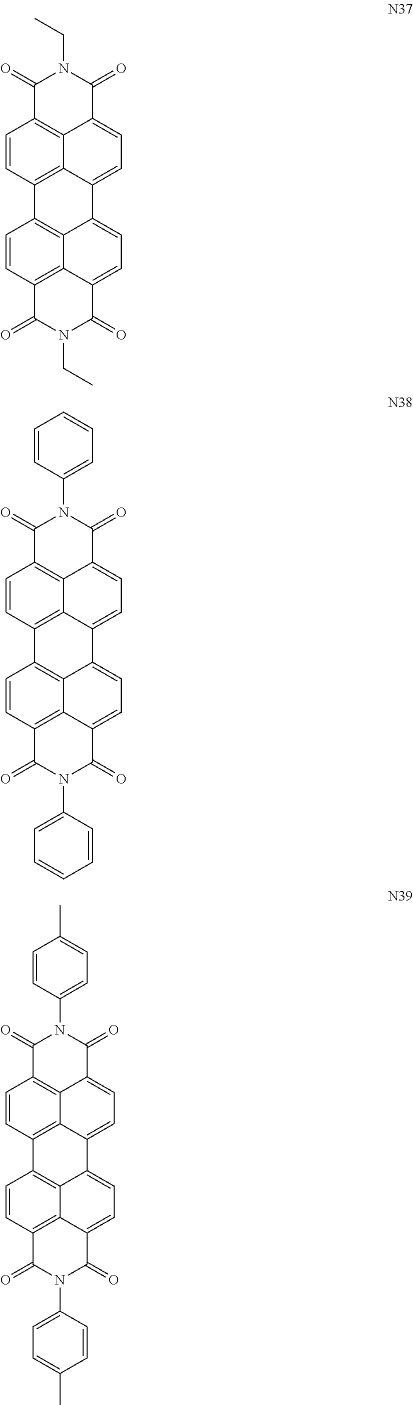

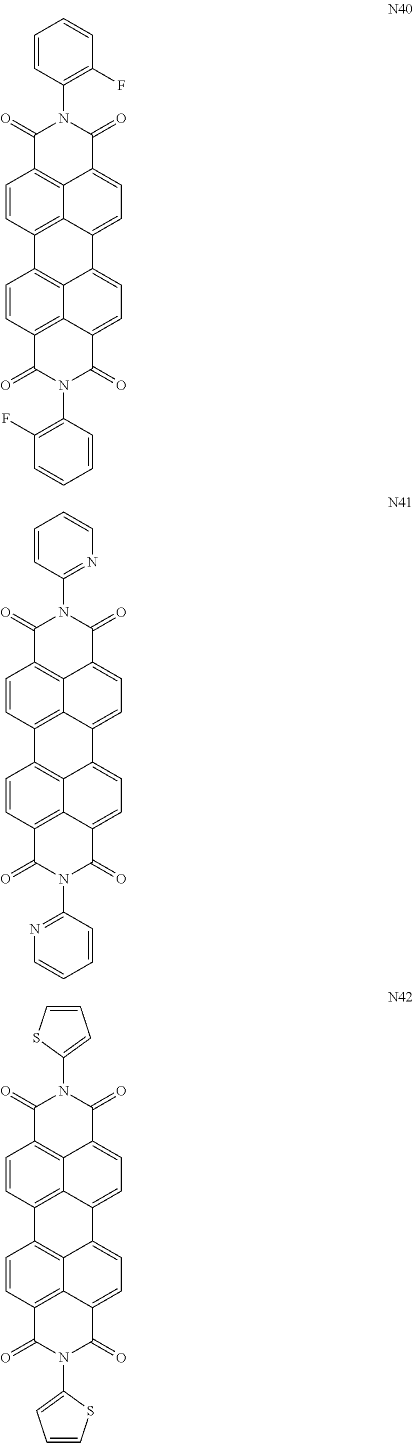

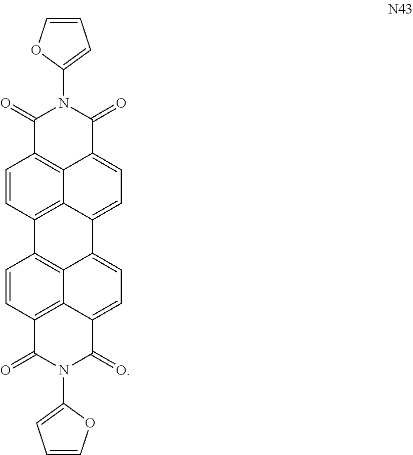 [SMILES: O=C1c2ccc3c4ccc5c6c(ccc(c7ccc(c2c37)C(=O)N1c1ccco1)c64)C(=O)N(c1ccco1)C5=O]

In the light-receiving device according to one or more embodiments,
the interlayer may further include an electron-acceptive layer,
the electron-acceptive layer may include the photoelectric conversion material, and
the electron-acceptive layer may be in direct contact with the light-receiving layer.

In one or more embodiments, an electronic apparatus may include the light-receiving device according to one or more embodiments of the present disclosure.

The electronic apparatus according to one or more embodiments may further include a thin-film transistor,
the thin-film transistor may include a source electrode and a drain electrode, and
the first electrode of the light-receiving device may be electrically connected to the source electrode or the drain electrode of the thin-film transistor.

The electronic apparatus according to one or more embodiments may further include a color filter, a color conversion layer, a touch screen layer, a polarizing layer, a light-guiding layer, a light-blocking layer, or any combination thereof. The light-guiding layer and the light-blocking layer may be formed on an identical layer, and may form an arbitrary light-guiding pattern and/or an arbitrary light-blocking pattern.

In one or more embodiments, an electronic device may include the light-receiving device according to one or more embodiments, and the electronic device may be a display including the light-receiving device. For example, the electronic device may be a flat panel display, a curved display, a computer monitor, a medical monitor, a television, a billboard, an indoor or outdoor lighting and/or signaling light, a head-up display, a fully or partially transparent display, a flexible display, a rollable display, a foldable display, a stretchable display, a microdisplay, a three-dimensional (3D) display, or a virtual reality or augmented reality display.

In some embodiments, the electronic device may be a product that may include a display, such as a laser printer, a telephone, a mobile phone, a tablet, a phablet, a personal digital assistant (PDA), a biometric authentication device, a wearable device, a laptop computer, a digital camera, a camcorder, a viewfinder, a vehicle, a video wall with multiple displays tiled together, a theater or stadium screen, a phototherapy device, or a signboard.

The condensed cyclic compound according to one or more embodiments may include at least one first repeating unit and at least one second repeating unit, and the first repeating unit may include at least one π electron-rich C₁-C₆₀ group. The second repeating unit may function as an electron-withdrawing group, and the π electron-rich C₁-C₆₀ group may function as an electron-donating group. In some embodiments, the first repeating unit may extend a conjugation system included in the second repeating unit and/or the π electron-rich C₁-C₆₀ group.

In some embodiments, due to the inclusion of the first repeating unit, direct bonding between the second repeating unit and the π electron-rich C₁-C₆₀ group may be limited, and thus, bonding of electrons and holes formed in the condensed cyclic compound may be delayed, and the condensed cyclic compound may provide improved stability.

In some embodiments, due to the inclusion of the first repeating unit between the second repeating unit and the π electron-rich C₁-C₆₀ group, the conjugation system included in the second repeating unit and/or the π electron-rich C₁-C₆₀ group may be extended. For example, as shown in Resonance structure 1, an unshared electron pair and/or a π electron pair of the π electron-rich C₁-C₆₀ group (indicated by EDG) may be delocalized in the second repeating unit, and the conjugation system formed in the second repeating unit may be extended by at least two allyl moieties. In Resonance Structure 1, EWG may correspond to R₇ and R₈ of Formulae 2-1 and 2-2.

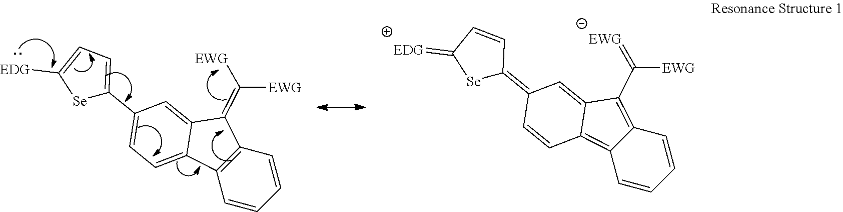

In some embodiments, by changing aspects of linkage between the first repeating unit and the second repeating unit included in the condensed cyclic compound of the present disclosure, a linking site of the first repeating unit and/or a linking site of the second repeating unit, and the type or kind and a linking site of the π electron-rich C₁-C₆₀ group, physical properties, such as the light-absorption wavelength, of the condensed cyclic compound may be adjusted, and the thermal stability and deposition stability thereof may be improved.

In particular, an increase in the length of a conjugation system may lead to an increase in the HOMO energy level and a decrease in the LUMO energy level of the condensed cyclic compound, and may further cause an increase in the maximum light-absorption wavelength (Amax) thereof.

For example, as shown in Resonance Structure 2, when the shortest conjugation system connecting an electron-donating group included in the π electron-rich C₁-C₆₀ group to R₇ of the second repeating unit includes 9 or more pairs of π electrons (including unshared electron pairs), the maximum light-absorption wavelength may be identified in the red wavelength region. It may be confirmed that the shortest conjugation system of the condensed cyclic compound shown in Resonance Structure 2 includes a total of 9 pairs of π electron pairs and unshared electron pairs.

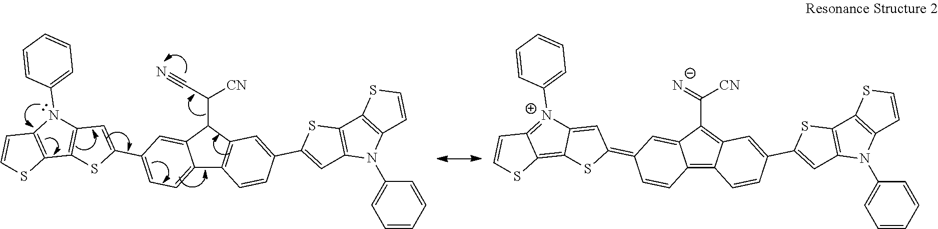 [SMILES: N#CC(C#N)C1c2cc(-c3cc4c(s3)c3sccc3n4-c3ccccc3)ccc2-c2ccc(-c3cc4c(s3)c3sccc3n4-c3ccccc3)cc21 ; N#CC(=[N-])C1=c2cc(-c3cc4c(s3)c3sccc3n4-c3ccccc3)ccc2=c2cc/c(=c3/cc4c(s3)=c3sccc3=[N+]4c3ccccc3)cc21]

In some embodiments, as shown in Resonance Structure 3, when the shortest conjugation system connecting the π electron-rich C₁-C₆₀ group to R₇ of the second repeating unit includes less than 9 pairs of π electrons (including unshared electron pairs), the maximum light-absorption wavelength may be identified in the green wavelength region. It may be confirmed that the shortest conjugation system of the condensed cyclic compound shown in Resonance Structure 3 includes a total of 8 pairs of π electron pairs and unshared electron pairs.

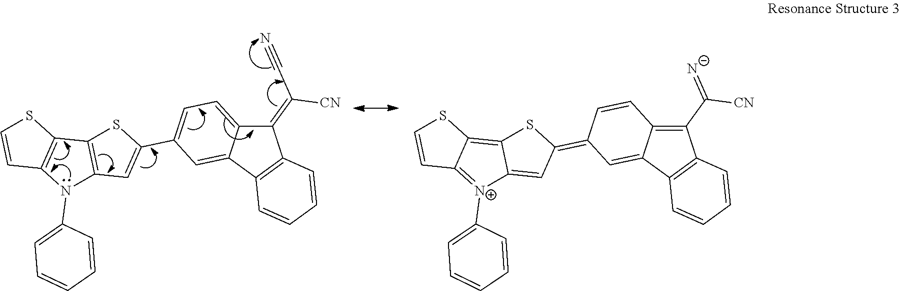 [SMILES: N#CC(C#N)=C1c2ccccc2-c2cc(-c3cc4c(s3)c3sccc3n4-c3ccccc3)ccc21 ; N#CC(=[N-])C1=c2cc/c(=c3/cc4c(s3)=c3sccc3=[N+]4c3ccccc3)cc2-c2ccccc21]

In some embodiments, the electron-donating group included in the π electron-rich C₁-C₆₀ group may include a delocalizable σ electron pair, for example, a delocalizable π electron pair or a delocalizable unshared electron pair. When the σ electron pair is the only delocalizable electron pair of the electron-donating group, the conjugation system may be extended by hyperconjugation. When the electron-donating group includes a π electron pair or an unshared electron pair, extension of the conjugation system may be confirmed through the resonance structure as described above.

In determination of the shortest conjugation system, an unshared electron pair, a π electron pair, and a σ electron pair may have decreasing priority in the stated order as the starting point of the conjugation system. For example, when the electron-donating group includes both (e.g., simultaneously) an unshared electron pair and a π electron pair, the unshared electron pair becomes the starting point of the shortest conjugation system. Likewise, when the electron-donating group includes only a π electron pair and a σ electron pair, the π electron pair becomes the starting point of the conjugation system.

In some embodiments, when the electron-donating group includes two or more electron pairs of the same priority, the shortest conjugation system may be one in which the number of allyl moieties involved in resonance-induced delocalization is smaller.

As described above, the condensed cyclic compound of the present disclosure may further include the first repeating unit, thereby diversifying aspects of linkage between the second repeating unit and the π electron-rich C₁-C₆₀ group. As a result, the light-absorption wavelength of the condensed cyclic compound may be controlled or selected, and the thermal stability and deposition stability thereof may be improved.

In one or more embodiments, the light-receiving device of the present disclosure may include a first electrode (anode), a second electrode (cathode), and an interlayer between the first electrode and the second electrode and including a light-receiving layer. Furthermore, in some embodiments, the light-receiving device of the present disclosure may include: i) a hole transport region between the first electrode and the light-receiving layer and including a hole injection layer, a hole transport layer, an auxiliary layer, an electron-blocking layer, or any combination thereof; and ii) an electron transport region between the light-receiving layer and the second electrode and including a hole-blocking layer, an electron transport layer, an electron injection layer, or any combination thereof.

In some embodiments, because the light-receiving device of the present disclosure further includes the electron transport region and the hole transport region, electrons and holes formed in the light-receiving layer may be transferred more efficiently, and deterioration of the light-receiving layer due to absorption of light energy may be suppressed or reduced. As a result, the external quantum efficiency of the light-receiving device may be improved, and the lifespan of the light-receiving device may be improved.

In particular, the light-receiving device of the present disclosure may be to absorb green light and/or red light belonging to a relatively long wavelength range, and because light energy of green light and red light may be converted into electrical energy, side reactions and decomposition in the light-receiving layer due to absorption of high energy wavelengths may be suppressed or reduced. As a result, the lifespan of the light-receiving device may be further improved.

Synthesis methods of the condensed cyclic compound including the first repeating unit and the second repeating unit may be recognizable by one of ordinary skill in the art by referring to Synthesis Examples and/or Examples provided herein.

At least one of the condensed cyclic compounds including the first repeating unit and the second repeating unit may be utilized in a light-receiving device (e.g., an organic light-receiving device). Accordingly, provided is a light-receiving device including: a first electrode; a second electrode facing the first electrode; an interlayer between the first electrode and the second electrode and including a light-receiving layer; and the condensed cyclic compound as described herein.

The expression “(interlayer and/or capping layer) includes a condensed cyclic compound” as utilized herein may be understood as “(interlayer and/or capping layer) may include one kind or type of condensed cyclic compound represented by Formula 1 or two different kinds or types of condensed cyclic compounds, each represented by Formula 1.”

For example, the interlayer and/or the capping layer may include Compound 1 only as the condensed cyclic compound. In this regard, Compound 1 may be present in the light-receiving layer of the light-receiving device. In one or more embodiments, the interlayer may include Compound 1 and Compound 2 as the condensed cyclic compound. In this regard, Compound 1 and Compound 2 may be present in an identical layer (e.g., Compound 1 and Compound 2 may both (e.g., simultaneously) be present in the light-receiving layer), or may be present in different layers (e.g., Compound 1 may be present in the light-receiving layer, and Compound 2 may be present in the electron transport region).

The term “interlayer” as utilized herein refers to a single layer and/or all of a plurality of layers between the first electrode and the second electrode of the light-receiving device.

According to one or more aspects of embodiments of the present disclosure, provided is an electronic apparatus including the light-receiving device as described above. The electronic apparatus may further include a thin-film transistor. For example, the electronic apparatus may further include a thin-film transistor including a source electrode and a drain electrode, and the first electrode of the light-receiving device may be electrically connected to the source electrode or the drain electrode. The electronic apparatus may further include a color filter, a color conversion layer, a touch screen layer, a polarizing layer, or any combination thereof. For more details on the electronic apparatus, related descriptions provided herein may be referred to.

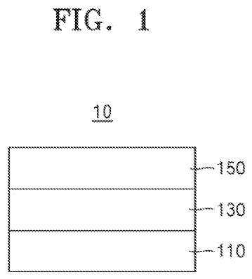

### DESCRIPTION OF FIG. 1

FIG. 1 is a schematic cross-sectional view of a light-receiving device 10 according to one or more embodiments of the present disclosure. The light-receiving device 10 may include a first electrode 110, an interlayer 130, and a second electrode 150.

Hereinafter, the structure of the light-receiving device 10 according to one or more embodiments and a method of manufacturing the light-receiving device 10 will be described with reference to FIG. 1.

### First Electrode 110

In FIG. 1, in some embodiments, a substrate may be additionally provided and arranged under the first electrode 110 or on the second electrode 150. As the substrate, a glass substrate or a plastic substrate may be utilized. In one or more embodiments, the substrate may be a flexible substrate, and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. In one or more embodiments, when the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure including (e.g., consisting of) a single layer or a multi-layered structure including a plurality of layers. For example, in some embodiments, the first electrode 110 may have a three-layered structure of ITO/Ag/ITO.

### Interlayer 130

The interlayer 130 may be on the first electrode 110. The interlayer 130 may include a light-receiving layer.

In one or more embodiments, the interlayer 130 may further include a hole transport region arranged between the first electrode 110 and the light-receiving layer and an electron transport region arranged between the light-receiving layer and the second electrode 150.

In one or more embodiments, the interlayer 130 may further include inorganic materials, such as quantum dots, in addition to one or more suitable organic materials.

In one or more embodiments, the interlayer 130 may include i) two or more light-receiving units sequentially stacked between the first electrode 110 and the second electrode 150, and ii) a transparent conductive layer arranged between the two or more light-receiving units. When the interlayer 130 includes the light-receiving units and the transparent conductive layer as described above, the light-receiving device 10 may be a tandem light-receiving device. For details on the transparent conductive layer, the description of the first electrode 110 may be referred to.

### Hole Transport Region in Interlayer 130

The hole transport region may have i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The hole transport region may include a hole injection layer, a hole transport layer, an auxiliary layer, an electron-blocking layer, or any combination thereof.

For example, in some embodiments, the hole transport region may have a multi-layered structure including a hole injection layer/hole transport layer structure, a hole injection layer/hole transport layer/auxiliary layer structure, a hole injection layer/auxiliary layer structure, a hole transport layer/auxiliary layer structure, or a hole injection layer/hole transport layer/electron-blocking layer structure, the layers of each structure being stacked sequentially from the first electrode 110 in the stated order.

The hole transport region may include a compound represented by Formula 201, a compound represented by Formula 202, or any combination thereof:

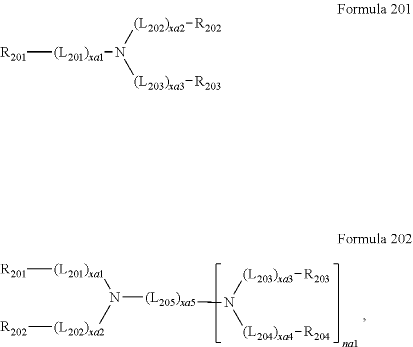

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀S may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (e.g., a carbazole group, etc.) unsubstituted or substituted with at least one R₁₀ₐ (e.g., Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, each of Formulae 201 and 202 may include at least one selected from groups represented by Formulae CY201 to CY217:

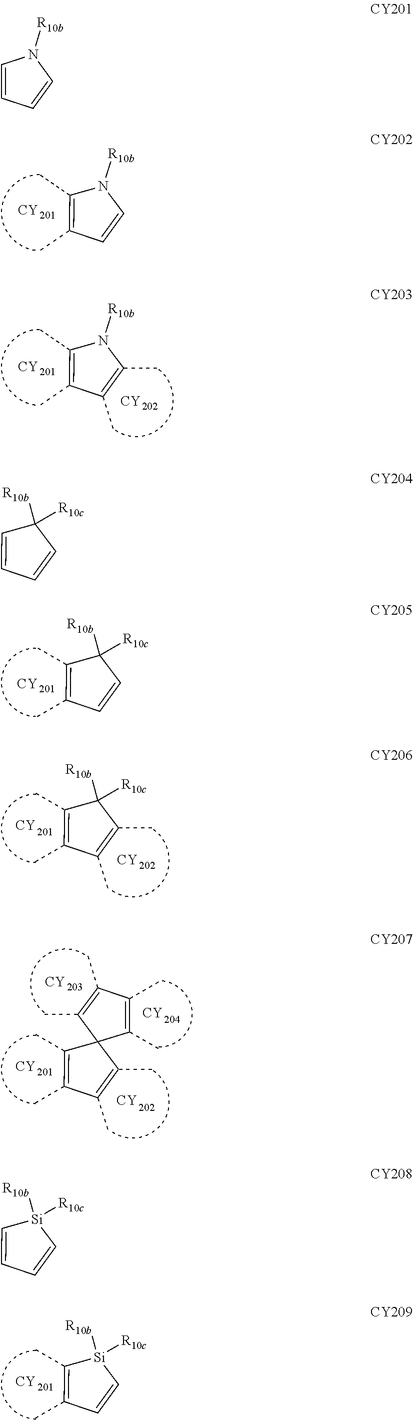

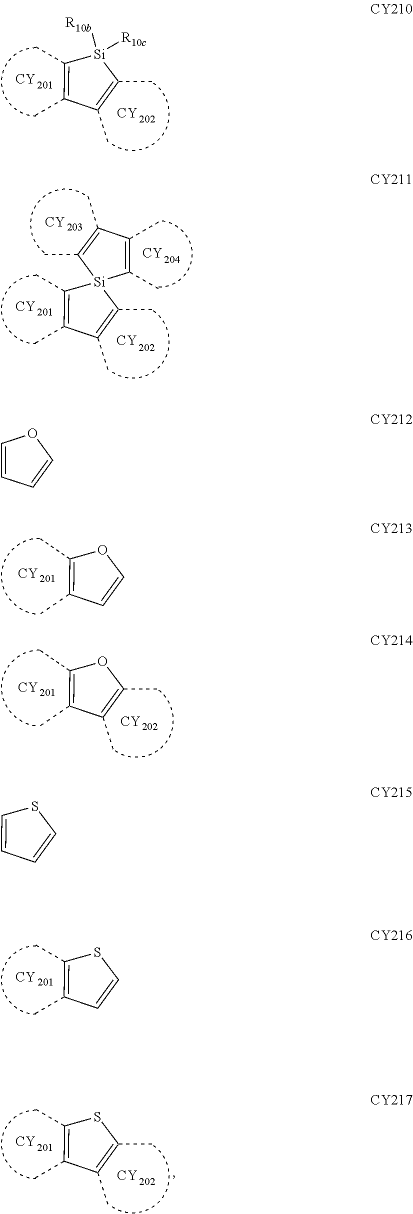

wherein, in Formulae CY201 to CY217, R_{10b} and R_{10c} may each be the same as described with respect to R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In one or more embodiments, ring CY201 to ring CY204 in Formulae CY201 to CY217 may each independently be a benzene group, a naphthalene group, a phenanthrene group, or an anthracene group.

In one or more embodiments, each of Formulae 201 and 202 may include at least one selected from groups represented by Formulae CY201 to CY203.

In one or more embodiments, Formula 201 may include at least one selected from the groups represented by Formulae CY201 to CY203 and at least one selected from groups represented by Formulae CY204 to CY217.

In one or more embodiments, in Formula 201, xa1 may be 1, R₂₀₁ may be a group represented by one selected from Formulae CY201 to CY203, xa2 may be 0, and R₂₀₂ may be a group represented by one selected from Formulae CY204 to CY207.

In one or more embodiments, each of Formulae 201 and 202 may not include (e.g., may exclude) a group represented by one selected from Formulae CY201 to CY203.

In one or more embodiments, each of Formulae 201 and 202 may not include (e.g., may exclude) a group represented by one selected from Formulae CY201 to CY203, and may include at least one selected from the groups represented by Formulae CY204 to CY217.

In one or more embodiments, each of Formulae 201 and 202 may not include (e.g., may exclude) a group represented by one selected from Formulae CY201 to CY217.

For example, in one or more embodiments, the hole transport region may include at least one selected from Compounds HT1 to HT46, 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), N,N′-di(1-naphthalene-1-yl)-N,N′-diphenyl-benzidine (NPB (NPD)), β-NPB, TPD, Spiro-TPD, Spiro-NPB, methylated NPB, 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), and/or any combination thereof:

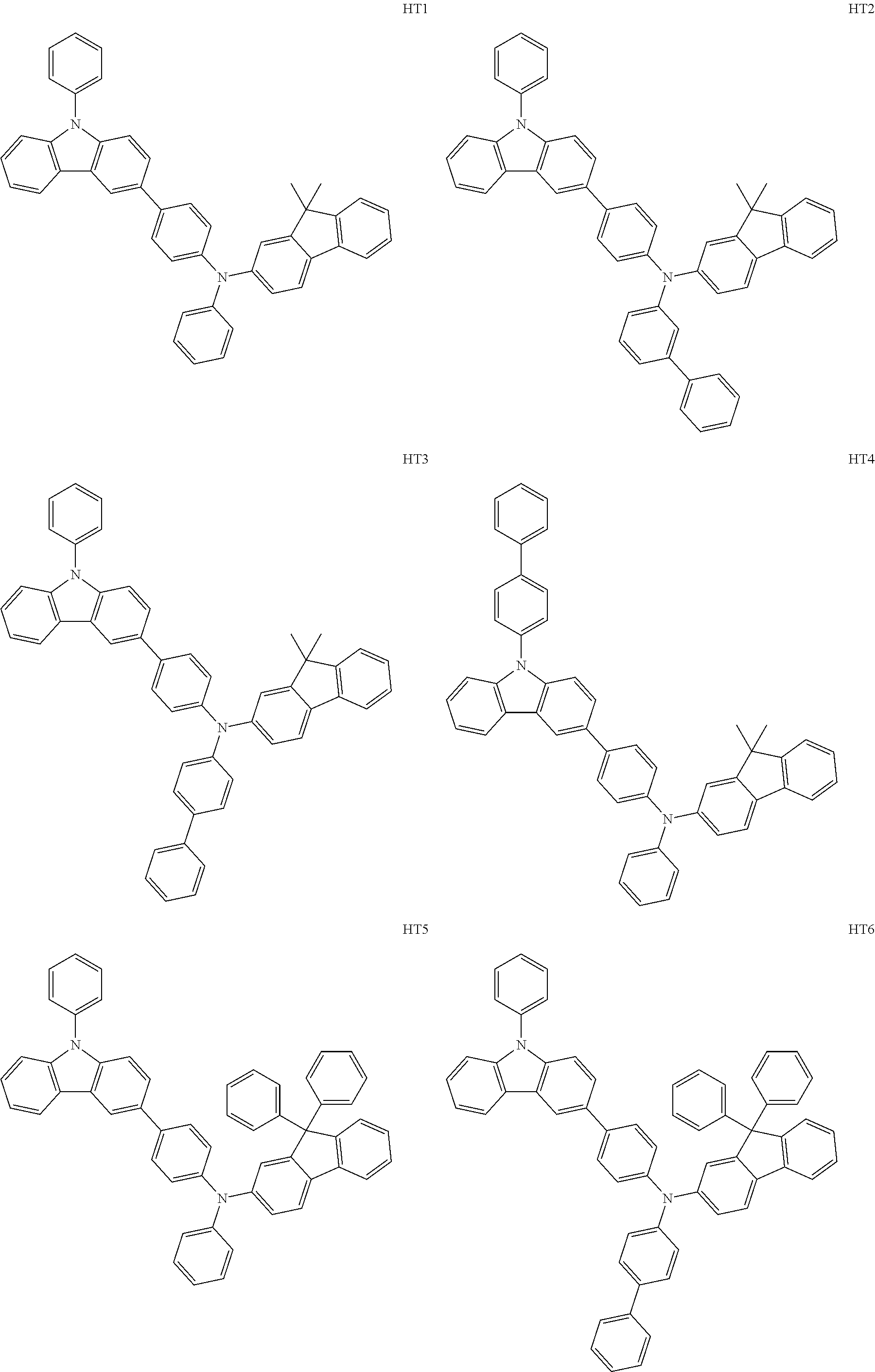

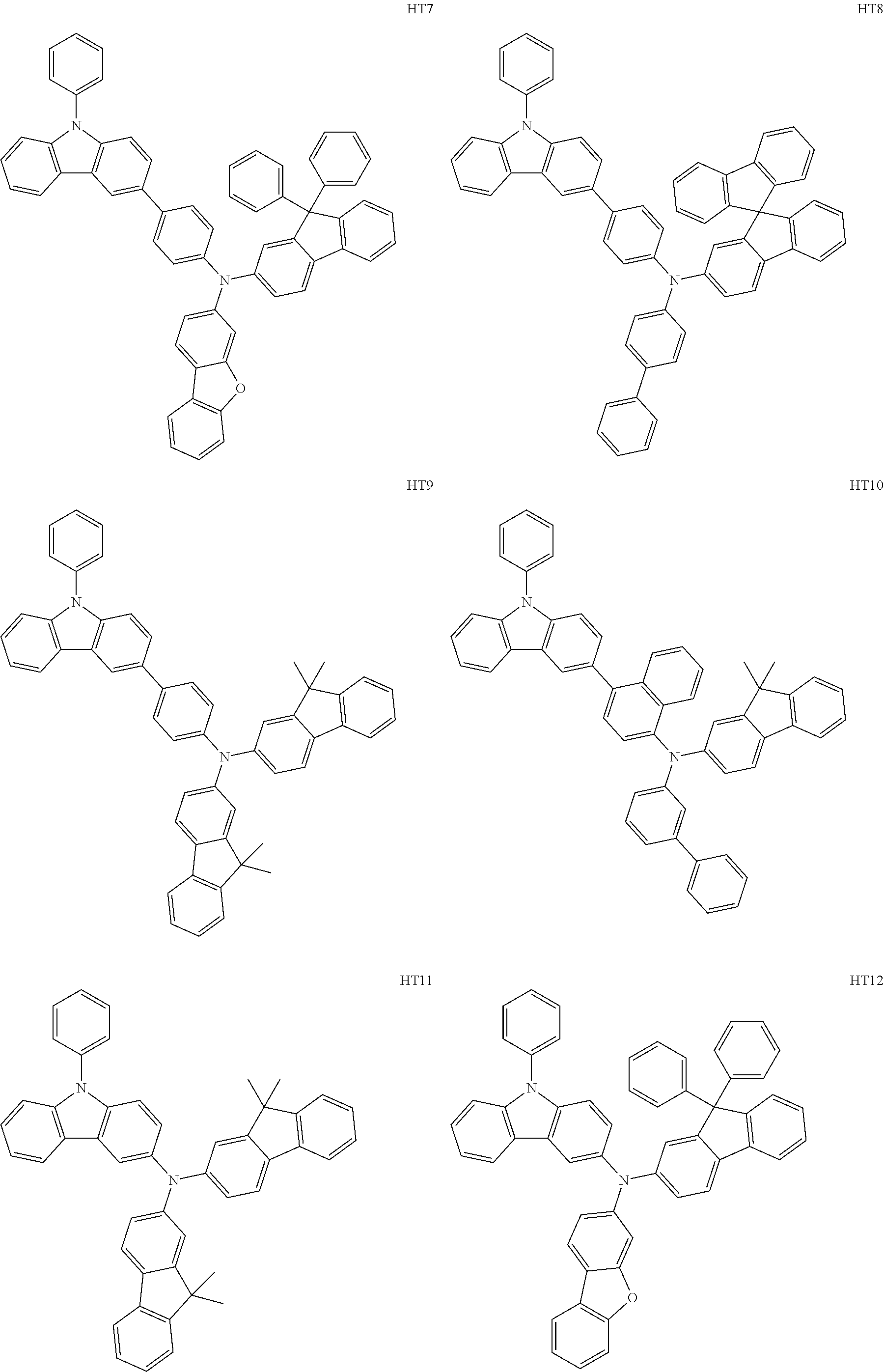

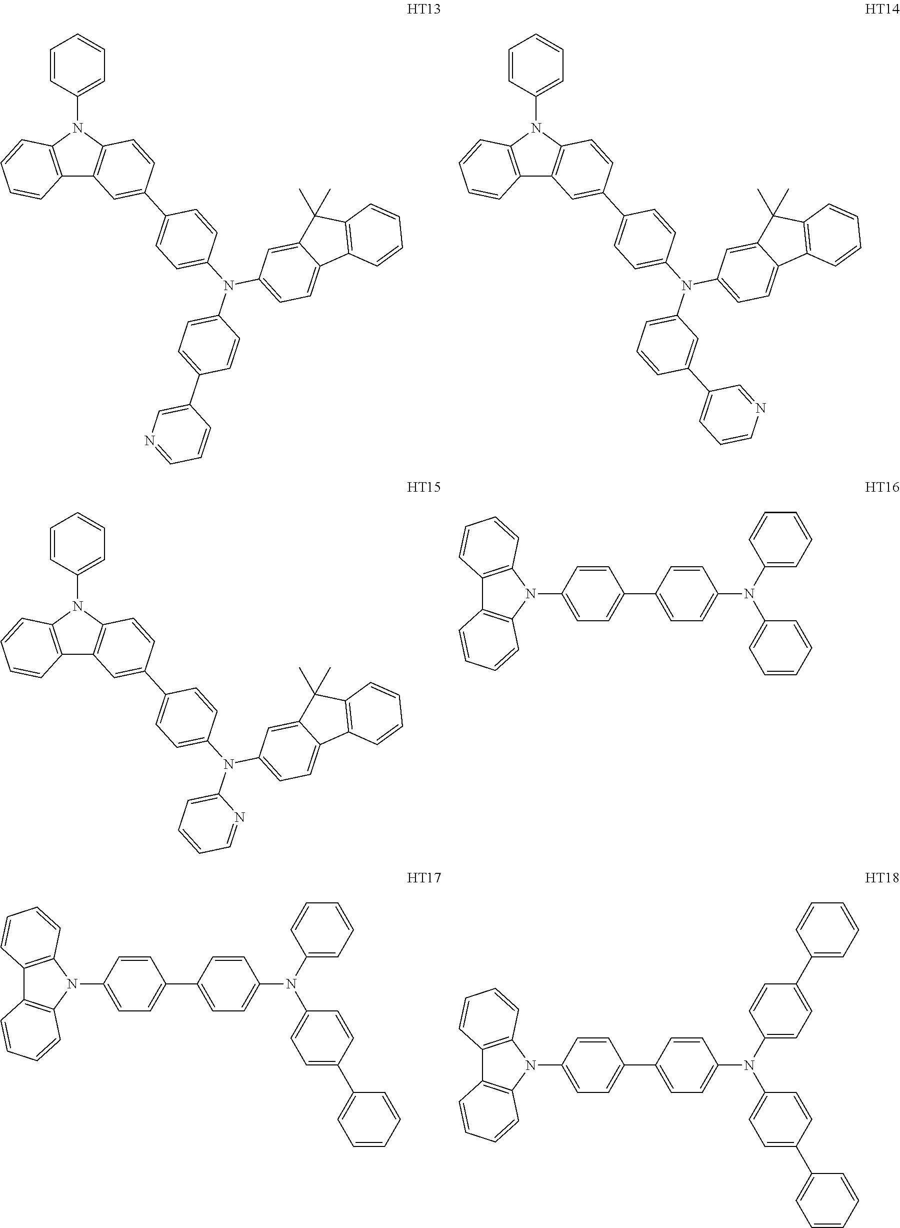 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

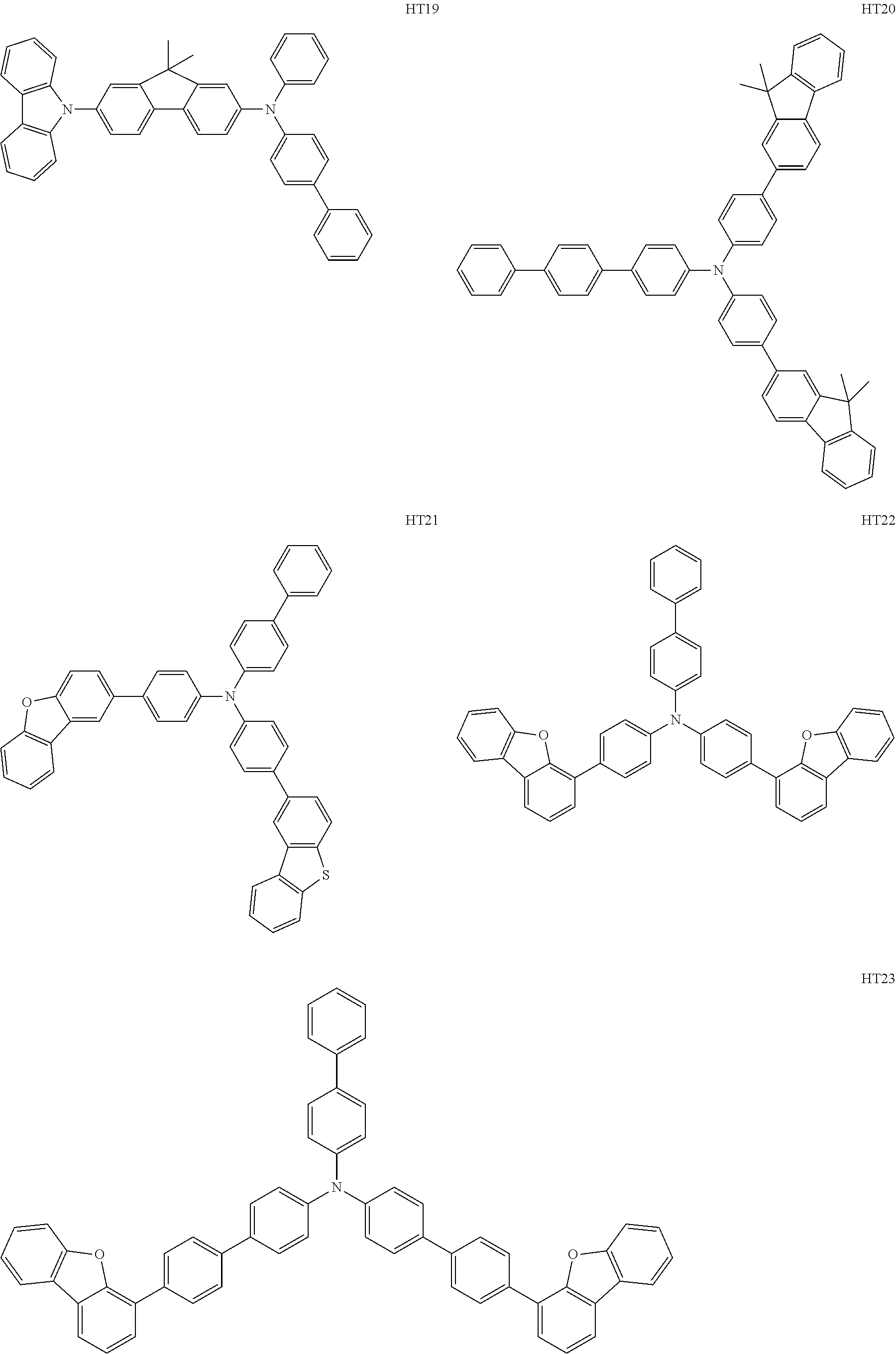 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

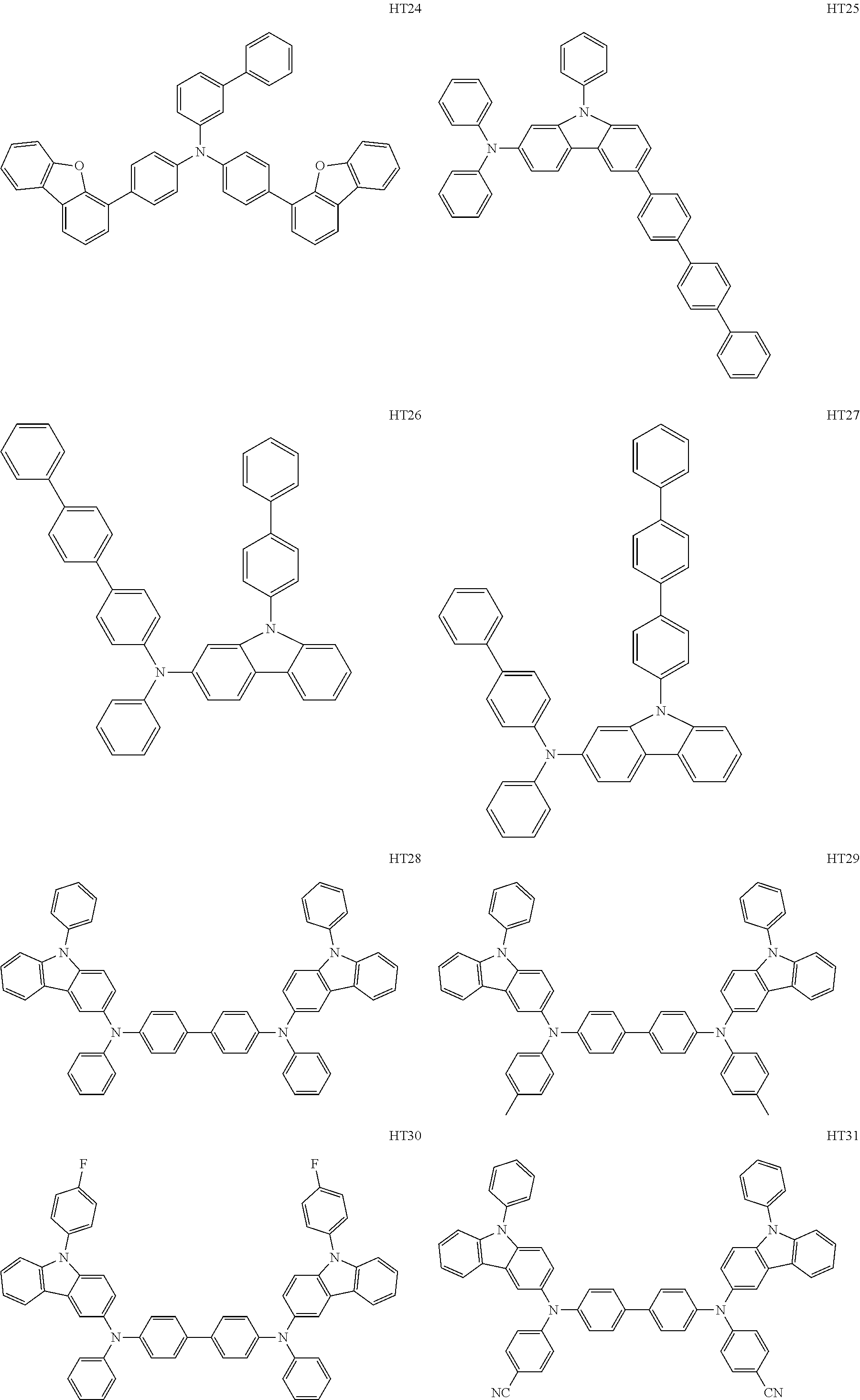

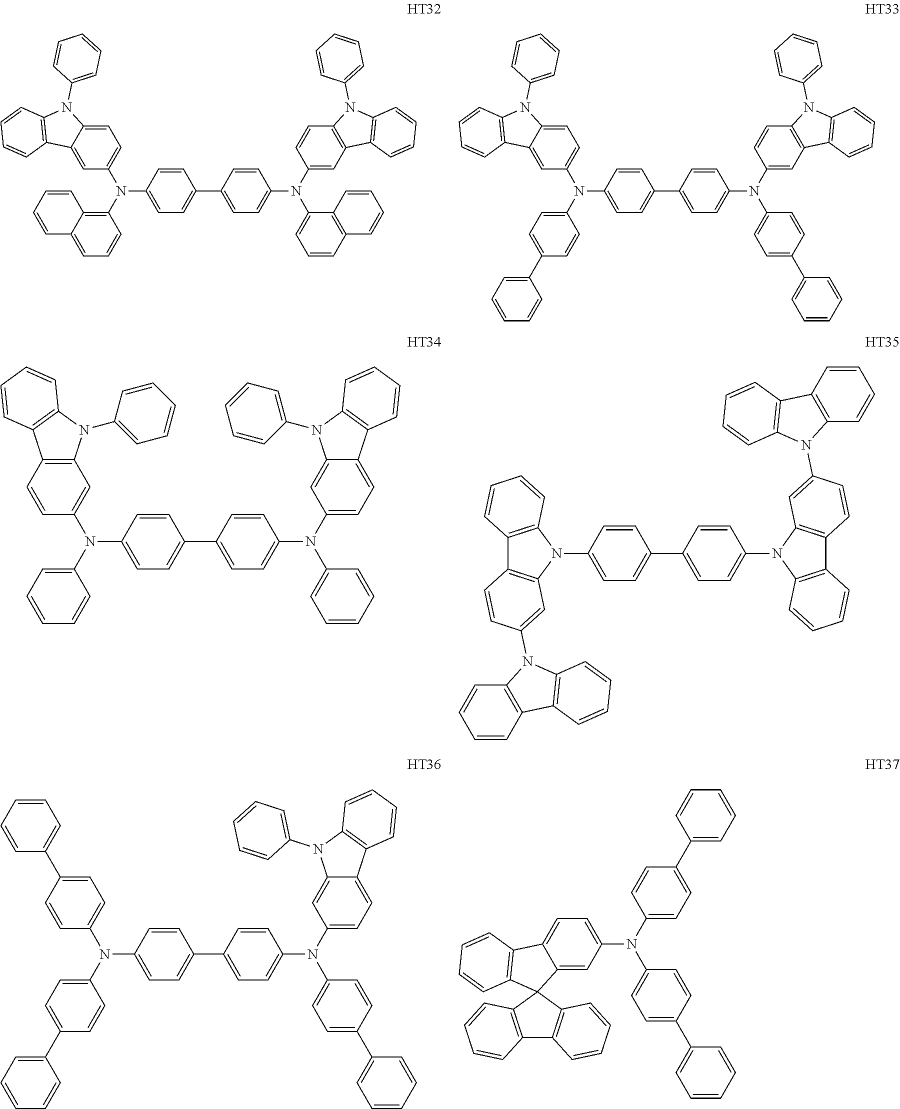

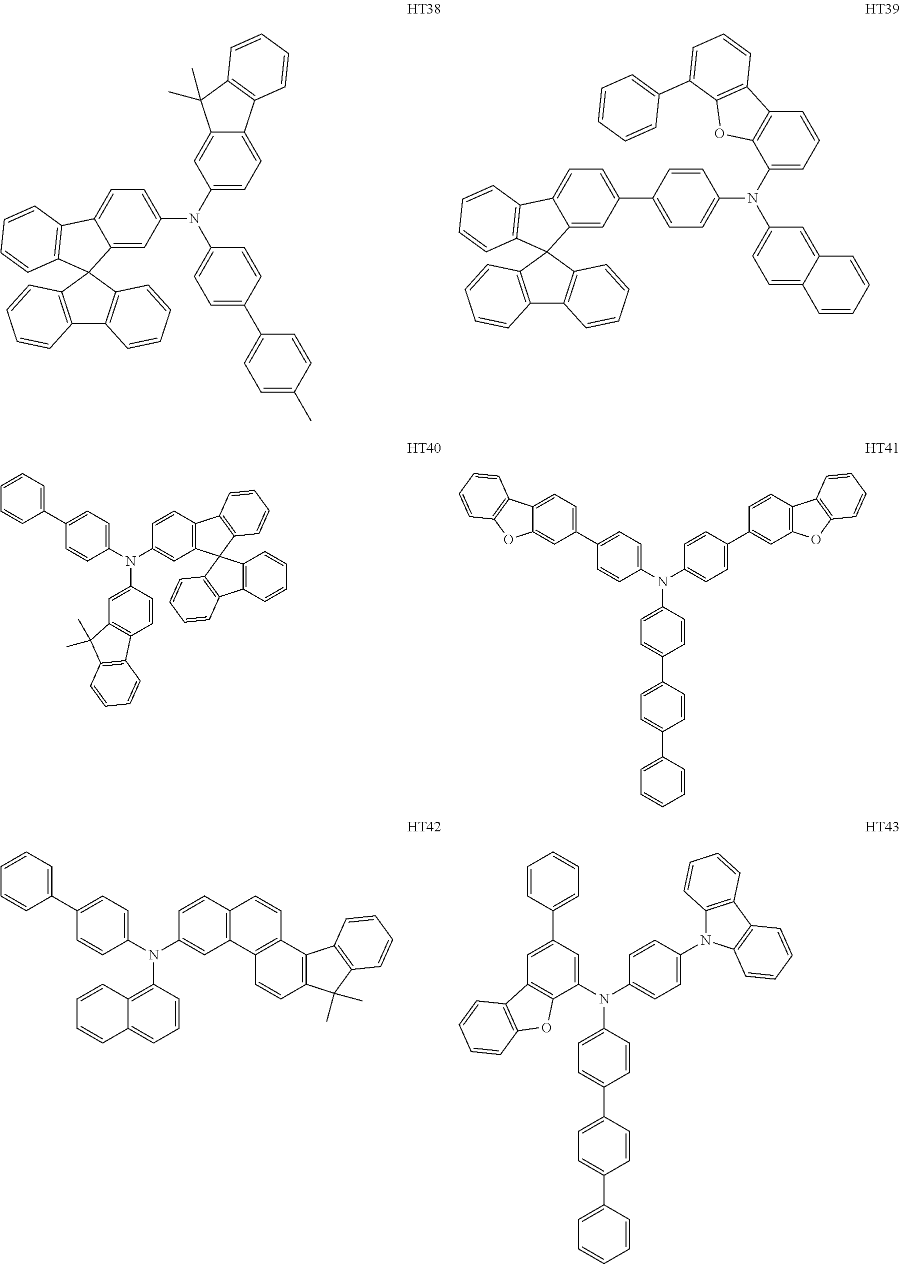

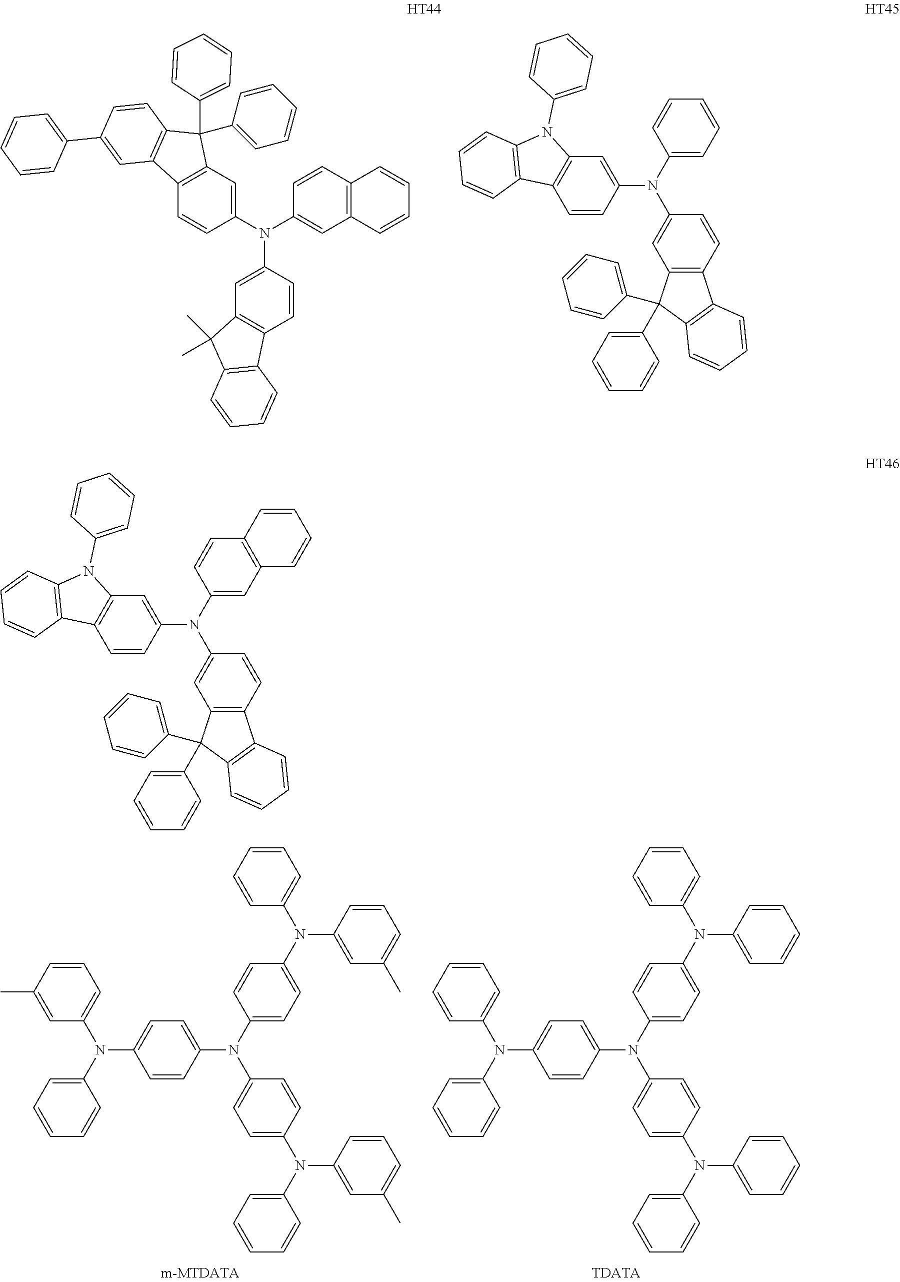

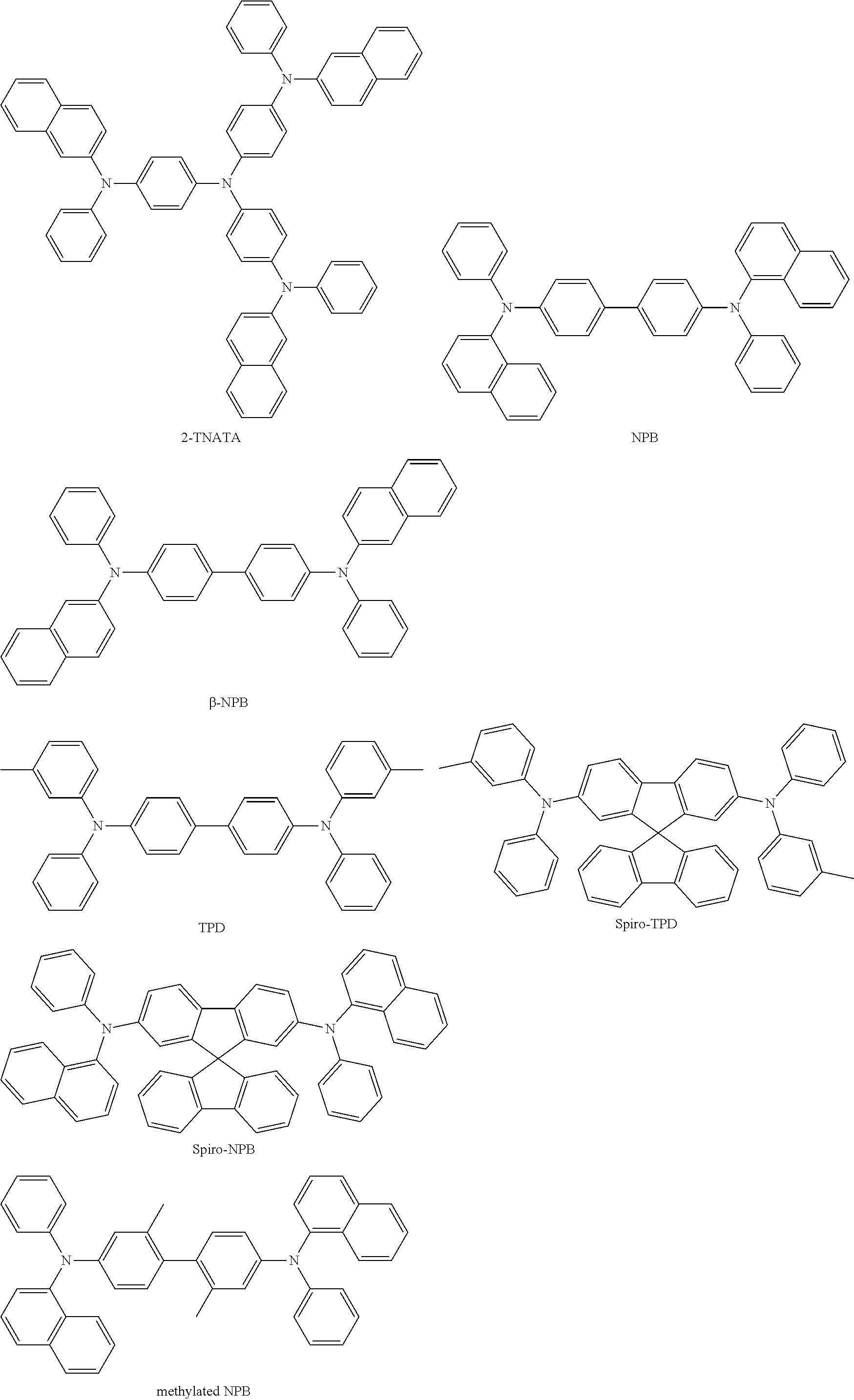

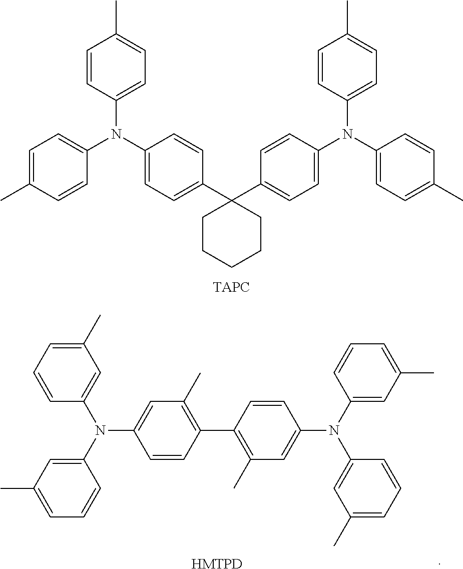

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The auxiliary layer may increase light-absorption efficiency by compensating for an optical resonance distance according to the wavelength of light absorbed by the light-receiving layer, and the electron-blocking layer may block or reduce the leakage of electrons from the light-receiving layer to the hole transport region. Materials that may be included in the hole transport region may be included in the auxiliary layer and the electron-blocking layer.

p-Dopant

In one or more embodiments, the hole transport region may further include, in addition to the materials as described above, a charge-generation material for improving conductive properties. The charge-generation material may be uniformly or non-uniformly dispersed in the hole transport region (e.g., in the form of a single layer including (e.g., consisting of) a charge-generation material).

The charge-generation material may be, for example, a p-dopant.

For example, the LUMO energy level of the p-dopant may be −3.5 eV or less.

In one or more embodiments, the p-dopant may include a quinone derivative, a cyano group-containing compound, a compound containing element EL1 and element EL2, or any combination thereof.

Non-limiting examples of the quinone derivative may include tetracyanoquinodimethane (TCNQ), 2,3,5,6-tetrafluoro-7,7,8,8-tetracyanoquinodimethane (F4-TCNQ), and/or the like.

Non-limiting examples of the cyano group-containing compound may include dipyrazino[2,3-f: 2′,3′-h] quinoxaline-2,3,6,7,10,11-hexacarbonitrile (HAT-CN), a compound represented by Formula 221, and/or the like:

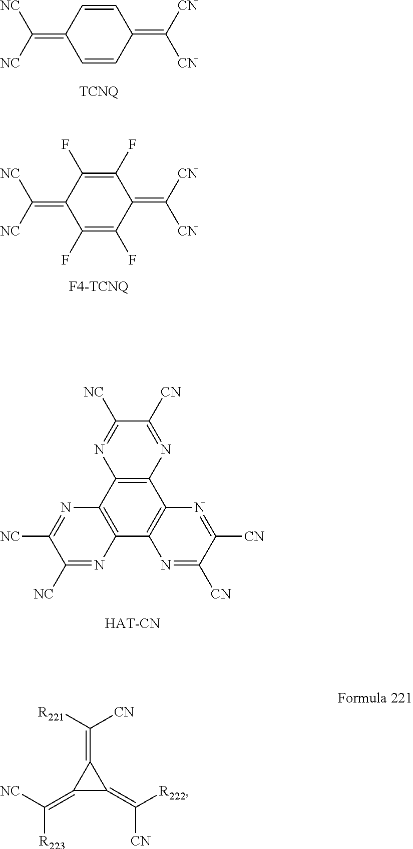

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one selected from among R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or any combination thereof, and element EL2 may be a non-metal, a metalloid, or any combination thereof.

Non-limiting examples of the metal may include: an alkali metal (e.g., lithium (Li), sodium (Na), potassium (K), rubidium (Rb), cesium (Cs), etc.); an alkaline earth metal (e.g., beryllium (Be), magnesium (Mg), calcium (Ca), strontium (Sr), barium (Ba), etc.); a transition metal (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (e.g., zinc (Zn), indium (In), tin (Sn), etc.); and/or a lanthanide metal (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Non-limiting examples of the metalloid may include silicon (Si), antimony (Sb), and/or tellurium (Te).

Non-limiting examples of the non-metal may include oxygen (O) and/or halogen (e.g., F, Cl, Br, I, etc.).

Non-limiting examples of the compound containing element EL1 and element EL2 may include a metal oxide, a metal halide (e.g., a metal fluoride, a metal chloride, a metal bromide, a metal iodide, etc.), a metalloid halide (e.g., a metalloid fluoride, a metalloid chloride, a metalloid bromide, a metalloid iodide, etc.), a metal telluride, and/or any combination thereof.

Non-limiting examples of the metal oxide may include a tungsten oxide (e.g., WO, W₂O₃, WO₂, WO₃, W₂O₅, etc.), a vanadium oxide (e.g., VO, V₂O₃, VO₂, V₂O₅, etc.), a molybdenum oxide (MoO, Mo₂O₃, MoO₂, MoO₃, Mo₂O₅, etc.), and/or a rhenium oxide (e.g., ReO₃, etc.).

Non-limiting examples of the metal halide may include an alkali metal halide, an alkaline earth metal halide, a transition metal halide, a post-transition metal halide, and/or a lanthanide metal halide.

Non-limiting examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and/or CsI.

Non-limiting examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, Bel₂, Mgl₂, Cal₂, SrI₂, and/or BaI₂.

Non-limiting examples of the transition metal halide may include a titanium halide (e.g., TiF₄, TiCl₄, TiBr₄, TiI₄, etc.), a zirconium halide (e.g., ZrF₄, ZrC₁₄, ZrBr₄, Zr₁₄, etc.), a hafnium halide (e.g., HfF₄, HfCl₄, HfBr₄, Hfl₄, etc.), a vanadium halide (e.g., VF₃, VCl₃, VBr₃, VI₃, etc.), a niobium halide (e.g., NbF₃, NbCl₃, NbBr₃, NbI₃, etc.), a tantalum halide (e.g., TaF₃, TaCl₃, TaBr₃, Ta₁₃, etc.), a chromium halide (e.g., CrF₃, CrCl₃, CrBr₃, CrI₃, etc.), a molybdenum halide (e.g., MoF₃, MoCl₃, MoBr₃, Mol₃, etc.), a tungsten halide (e.g., WF₃, WCl₃, WBr₃, WI₃, etc.), a manganese halide (e.g., MnF₂, MnCl₂, MnBr₂, MnI₂, etc.), a technetium halide (e.g., TcF₂, TcCl₂, TcBr₂, TcI₂, etc.), a rhenium halide (e.g., ReF₂, ReCl₂, ReBr₂, ReI₂, etc.), an iron halide (e.g., FeF₂, FeCl₂, FeBr₂, FeI₂, etc.), a ruthenium halide (e.g., RuF₂, RuCl₂, RuBr₂, RuI₂, etc.), an osmium halide (e.g., OsF₂, OsCl₂, OsBr₂, OsI₂, etc.), a cobalt halide (e.g., CoF₂, COCl₂, CoBr₂, CoI₂, etc.), a rhodium halide (e.g., RhF₂, RhCl₂, RhBr₂, RhI₂, etc.), an iridium halide (e.g., IrF₂, IrCl₂, IrBr₂, IrI₂, etc.), a nickel halide (e.g., NiF₂, NiCl₂, NiBr₂, NiI₂, etc.), a palladium halide (e.g., PdF₂, PdCl₂, PdBr₂, PdI₂, etc.), a platinum halide (e.g., PtF₂, PtCl₂, PtBr₂, PtI₂, etc.), a copper halide (e.g., CuF, CuCl, CuBr, CuI, etc.), a silver halide (e.g., AgF, AgCl, AgBr, Agl, etc.), and/or a gold halide (e.g., AuF, AuCl, AuBr, AuI, etc.).

Non-limiting examples of the post-transition metal halide may include a zinc halide (e.g., ZnF₂, ZnCl₂, ZnBr₂, ZnI₂, etc.), an indium halide (e.g., Ink₃, etc.), and/or a tin halide (e.g., SnI₂, etc.).

Non-limiting examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, and/or SmI₃.

Non-limiting examples of the metalloid halide may include an antimony halide (e.g., SbCl₅, etc.).

Non-limiting examples of the metal telluride may include an alkali metal telluride (e.g., Li₂Te, Na₂Te, K₂Te, Rb₂Te, Cs₂Te, etc.), an alkaline earth metal telluride (e.g., BeTe, MgTe, CaTe, SrTe, BaTe, etc.), a transition metal telluride (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (e.g., ZnTe, etc.), and/or a lanthanide metal telluride (e.g., LaTe, CeTe, PrTe, NdTe, PmTe, EuTe, GdTe, TbTe, DyTe, HoTe, ErTe, TmTe, YbTe, LuTe, etc.).

### Light-Receiving Layer in Interlayer 130

When the light-receiving device 10 is a full-color light-receiving device, the light-receiving layer may be patterned into a red light-receiving layer, a green light-receiving layer, and/or a blue light-receiving layer, according to a sub-pixel. In one or more embodiments, the light-receiving layer may have a stacked structure of two or more layers selected from a red light-receiving layer, a green light-receiving layer, and a blue light-receiving layer, in which the two or more layers contact each other or are separated from each other to receive (e.g., absorb) white light. In one or more embodiments, the light-receiving layer may include two or more materials selected from a red light-receiving material, a green light-receiving material, and a blue light-receiving material, in which the two or more materials are mixed with each other in a single layer to receive white light.

The light-receiving layer may include a light-receiving compound. The light-receiving compound may include an organic compound, an inorganic compound, or an organic compound and an inorganic compound.

In some embodiments, the light-receiving compound included in the light-receiving layer may be the condensed cyclic compound including the first repeating unit and the second repeating unit.

An amount of the light-receiving compound in the light-receiving layer may be in a range of about 0.01 part by weight to about 15 parts by weight based on total 100 parts by weight of the light-receiving layer.

In one or more embodiments, the light-receiving layer may further include a quantum dot.

In one or more embodiments, the light-receiving layer may further include a delayed fluorescence material. The delayed fluorescence material may act as a host or a dopant in the light-receiving layer.

A thickness of the light-receiving layer may be in a range of about 100 Å to about 1,000 Å, for example, about 200 Å to about 600 Å. When the thickness of the light-receiving layer is within these ranges, excellent or suitable light-receiving characteristics may be obtained without substantial deterioration due to light absorption.

### Host

In one or more embodiments, the host may include a compound represented by Formula 301:

[Ar₃₀₁]_{xb11}-[(L₃₀₁)_{xb1}-R₃₀₁]_{xb21}, Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each be the same as described with respect to Q₁.

For example, in some embodiments, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

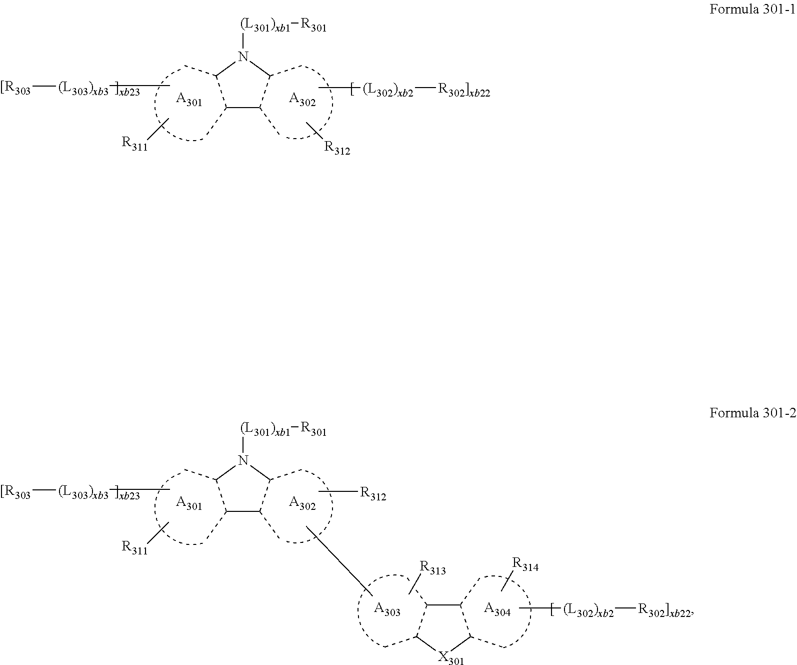

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)xb4-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be the same as described herein,
L₃₀₂ to L₃₀₄ may each independently be the same as described with respect to L₃₀₁,
xb2 to xb4 may each independently be the same as described with respect to xb1, and
R₃₀₂ to R₃₀₅ and R₃₁₁ to R₃₁₄ may each be the same as described with respect to R₃₀₁.

In one or more embodiments, the host may include an alkaline earth metal complex, a post-transition metal complex, or any combination thereof. For example, in some embodiments, the host may include a Be complex (e.g., Compound H55), an Mg complex, a Zn complex, or any combination thereof.

In one or more embodiments, the host may include at least one selected from among Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), and/or any combination thereof:

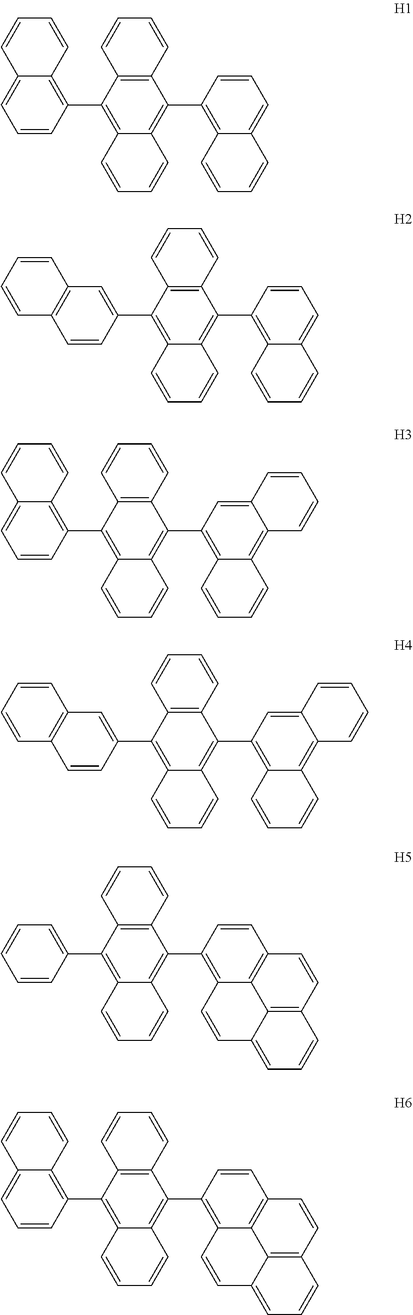

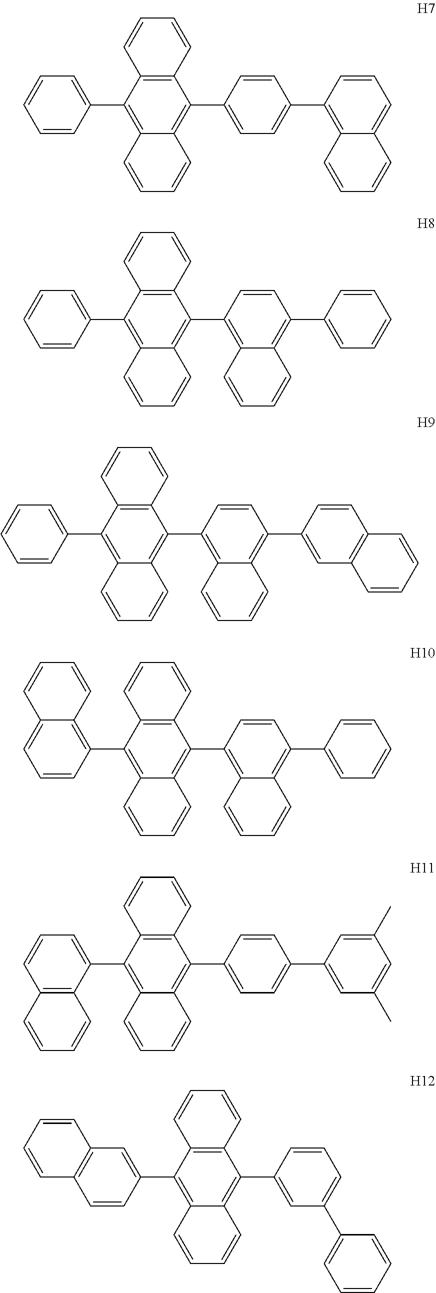

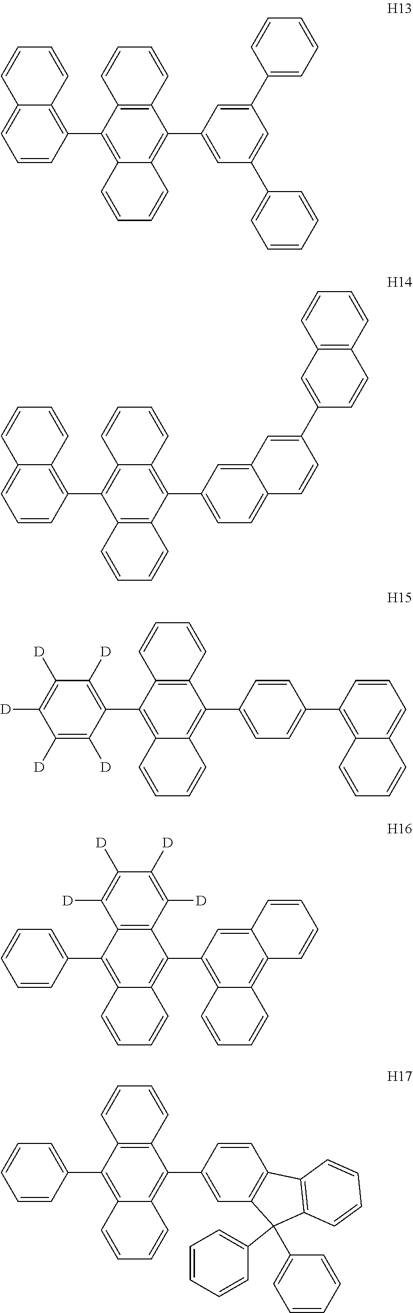

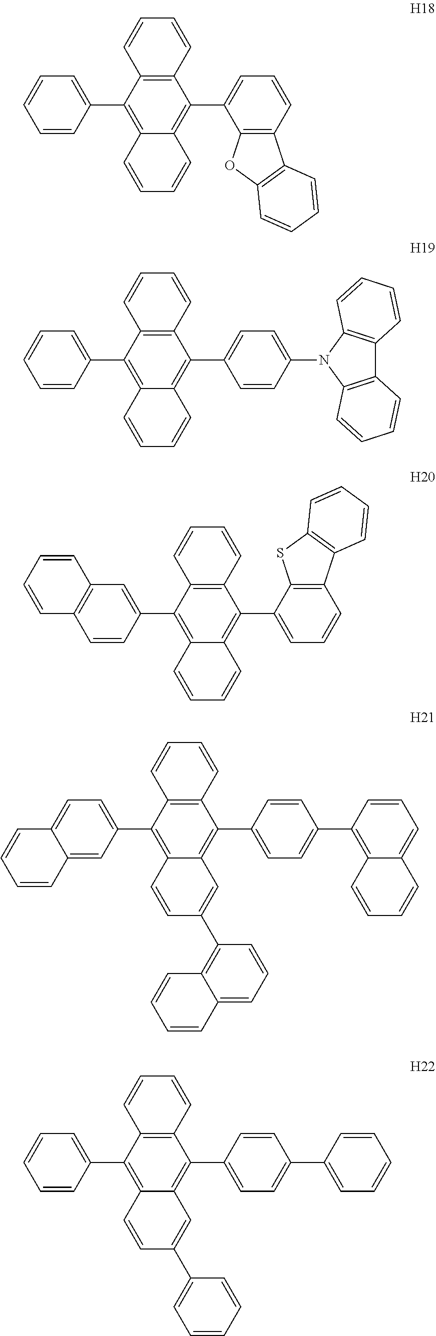

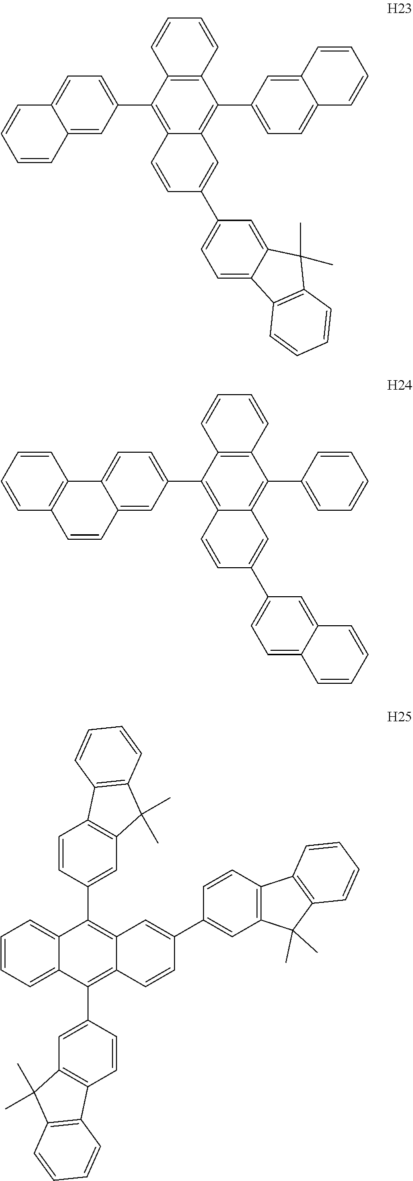

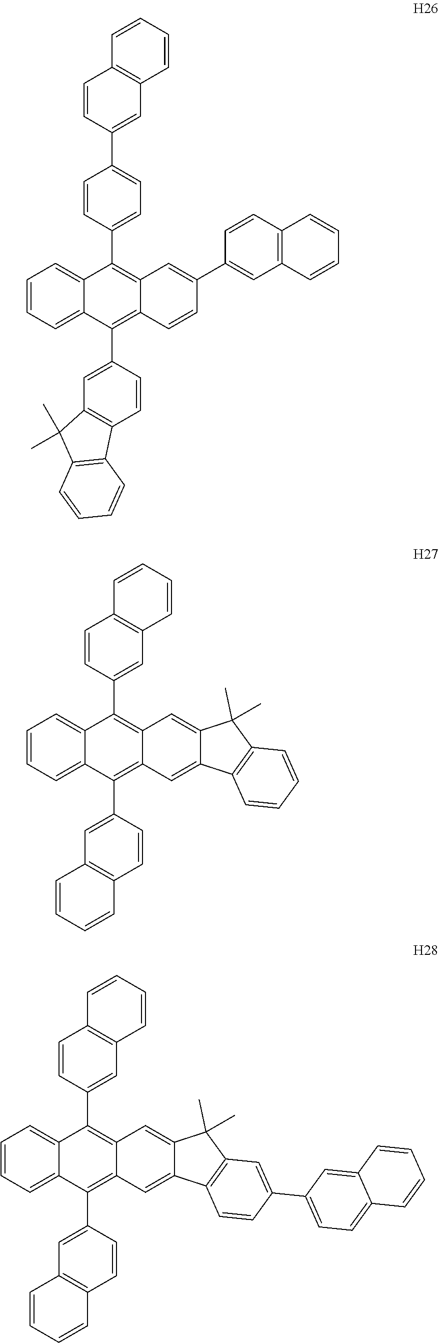

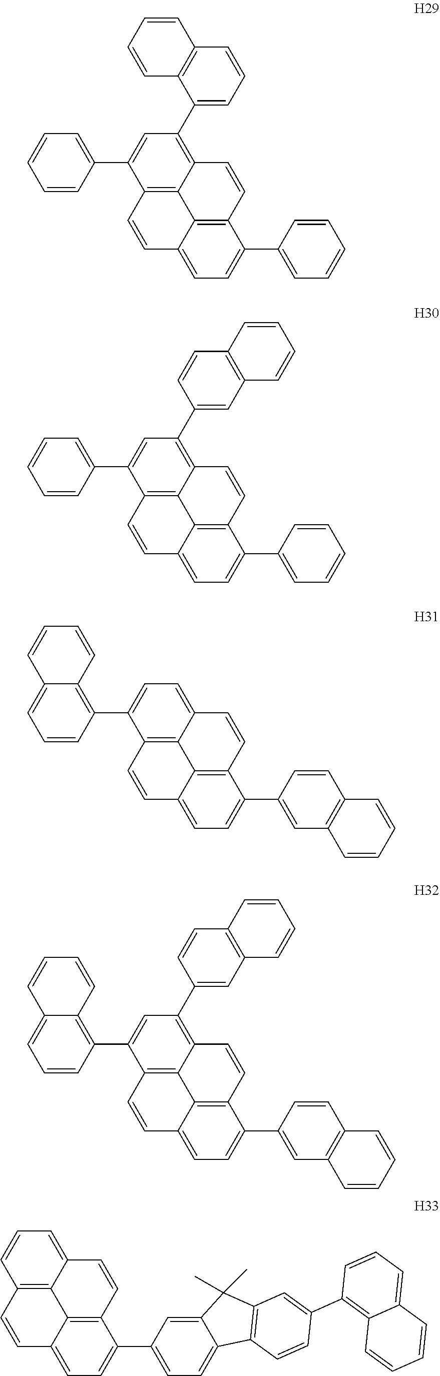

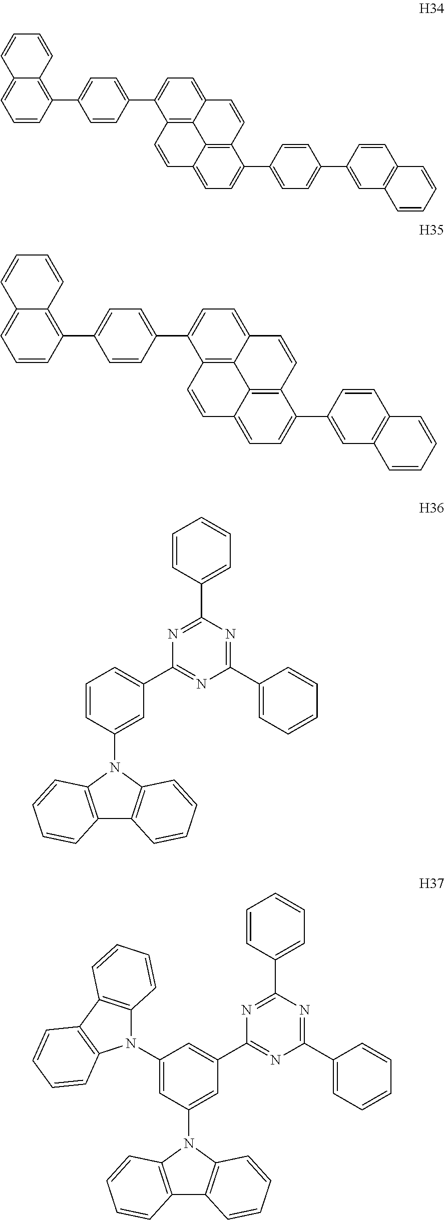

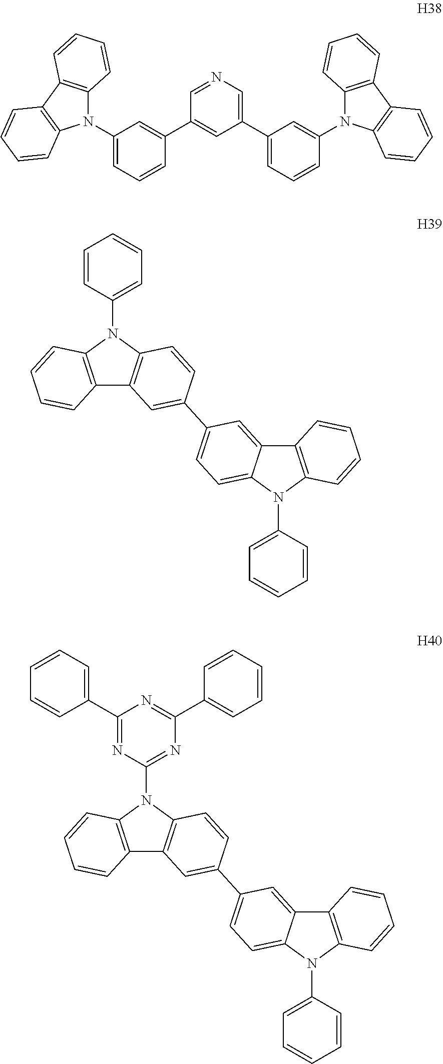 [SMILES: c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1]

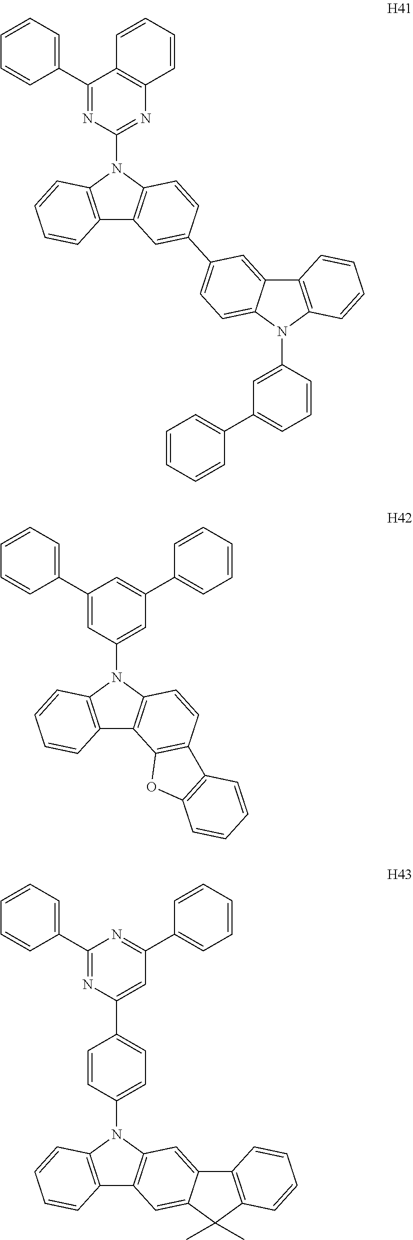 [SMILES: c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1 ; CC1(C)c2ccccc2-c2cc3c(cc21)c1ccccc1n3-c1ccc(-c2cc(-c3ccccc3)nc(-c3ccccc3)n2)cc1]

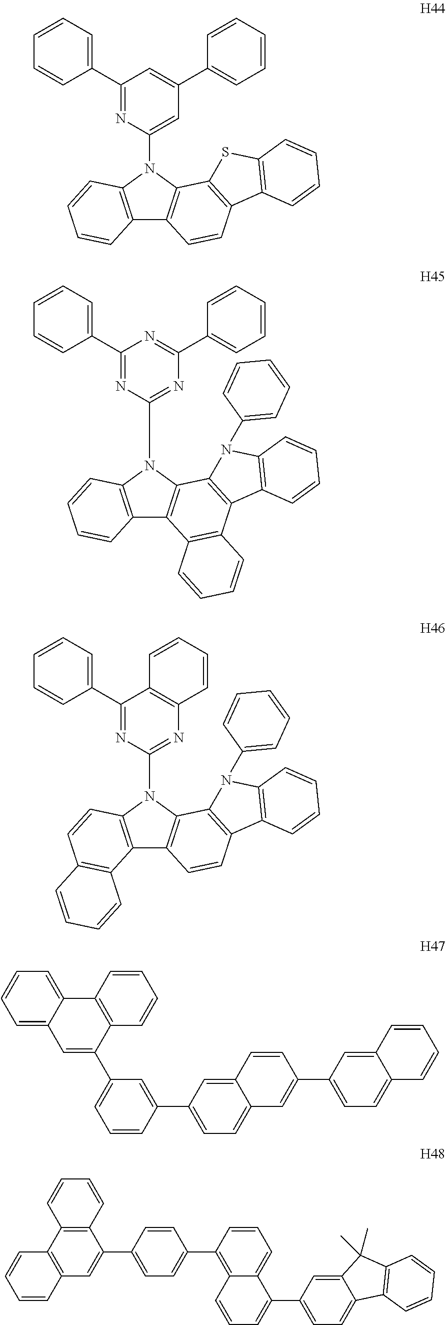

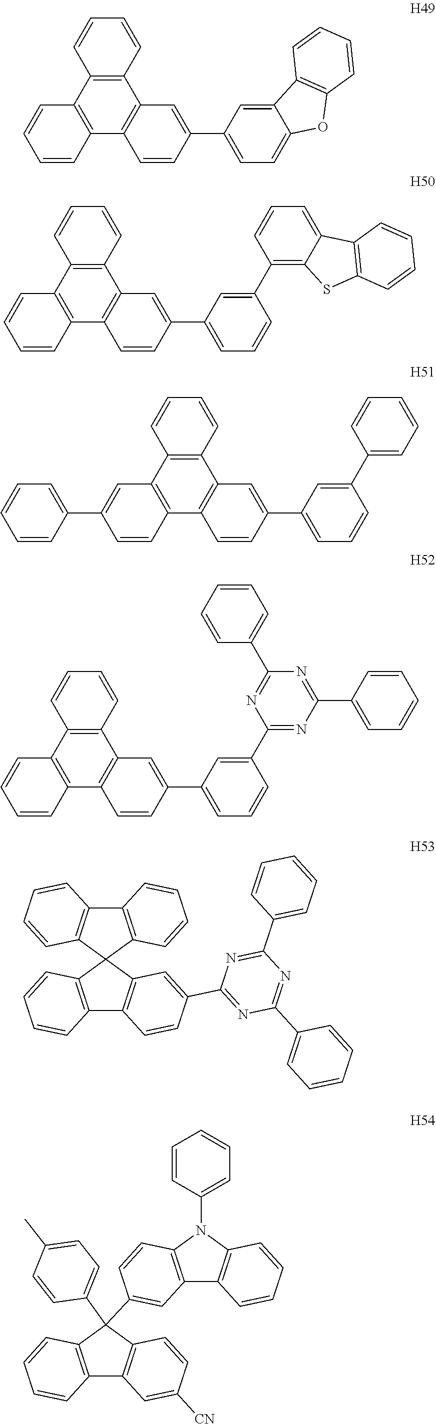

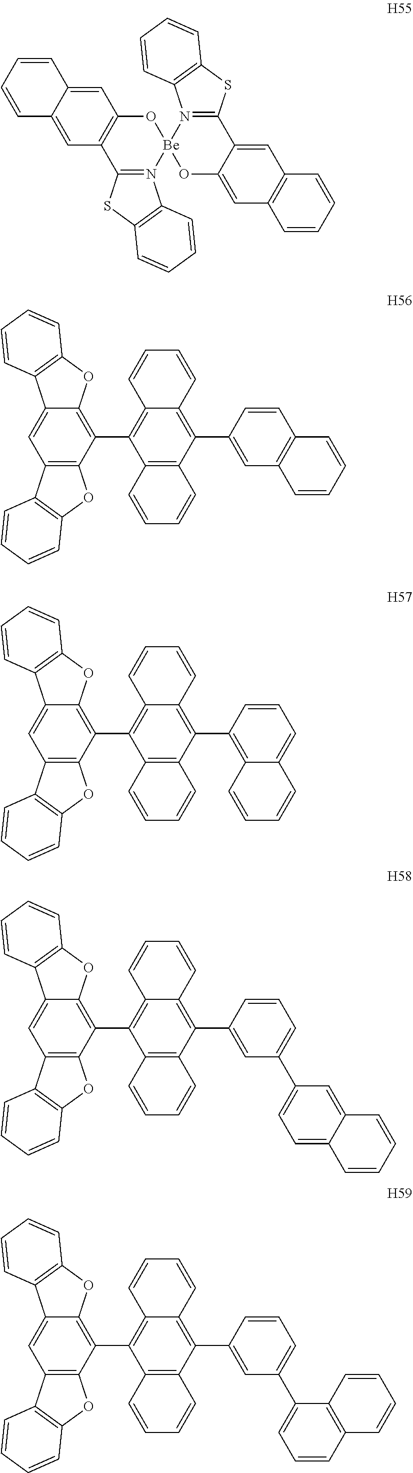

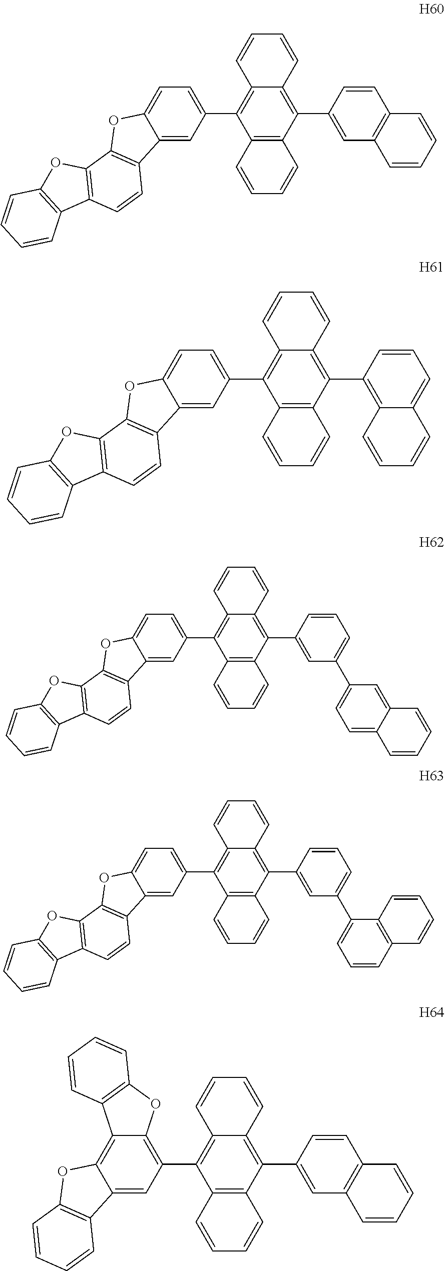 [SMILES: c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1]

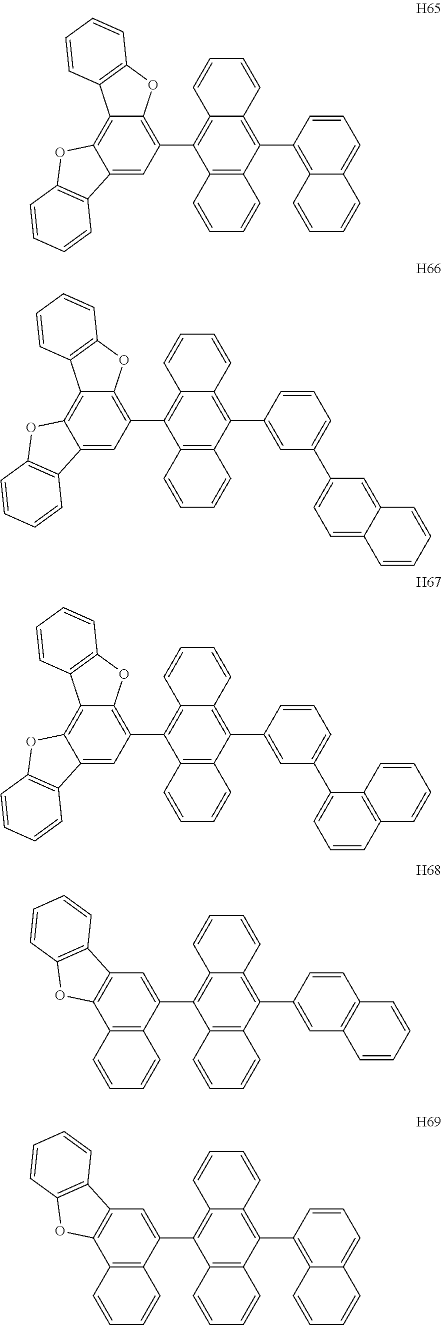

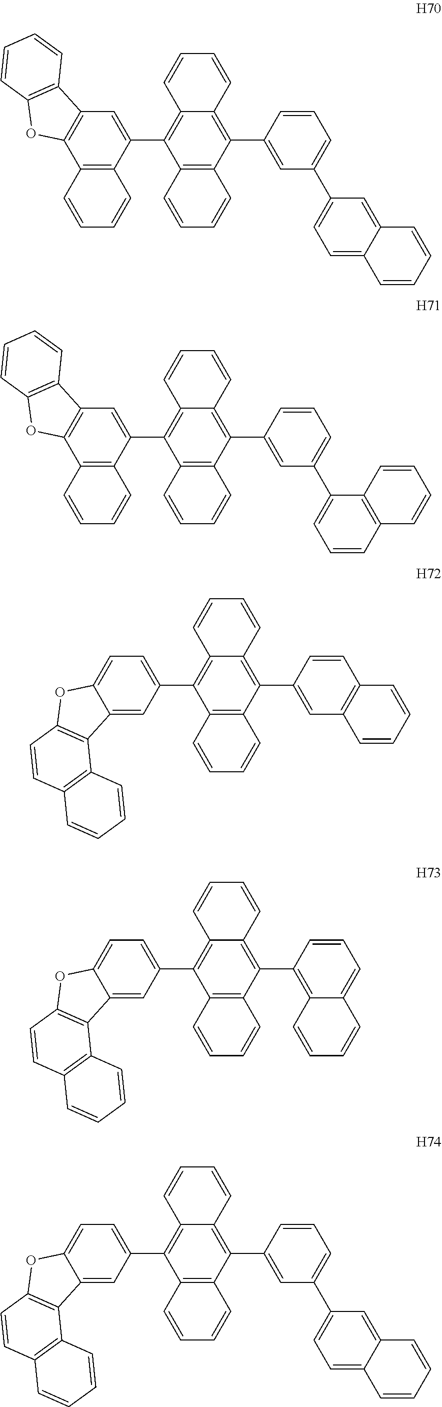 [SMILES: c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1 ; c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1]

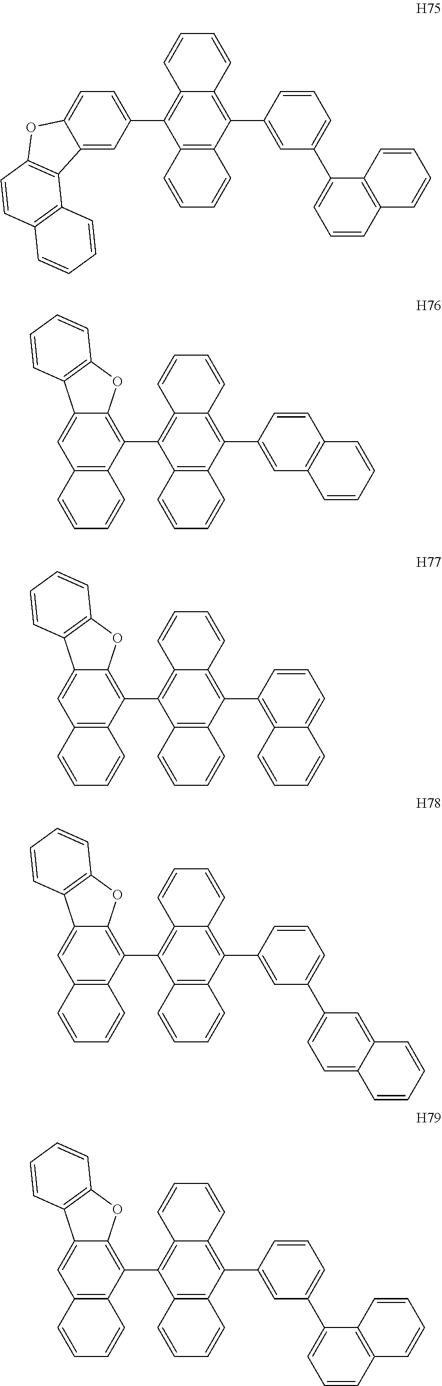

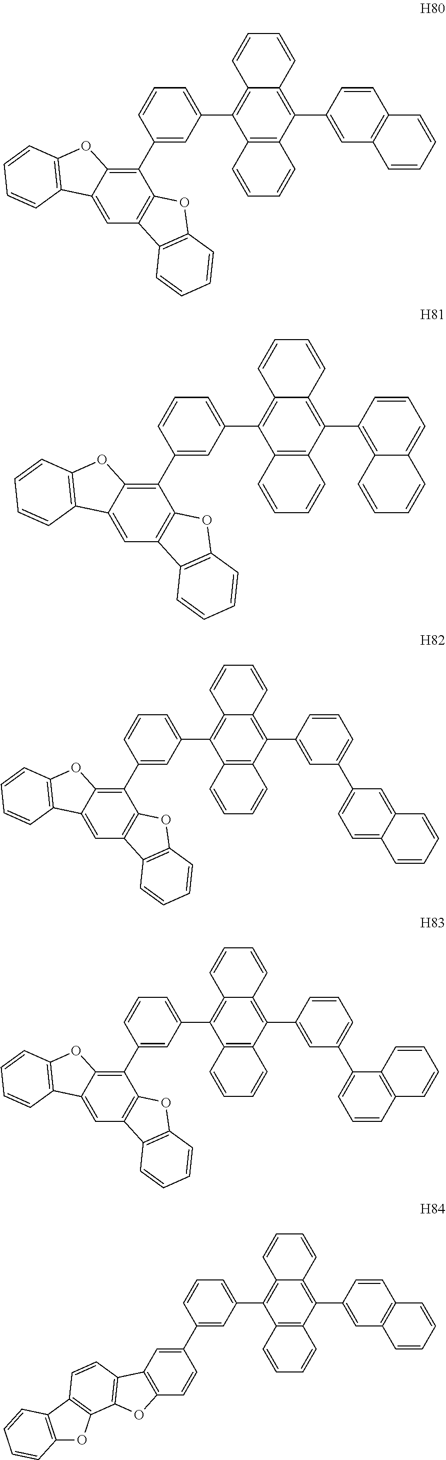 [SMILES: c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1]

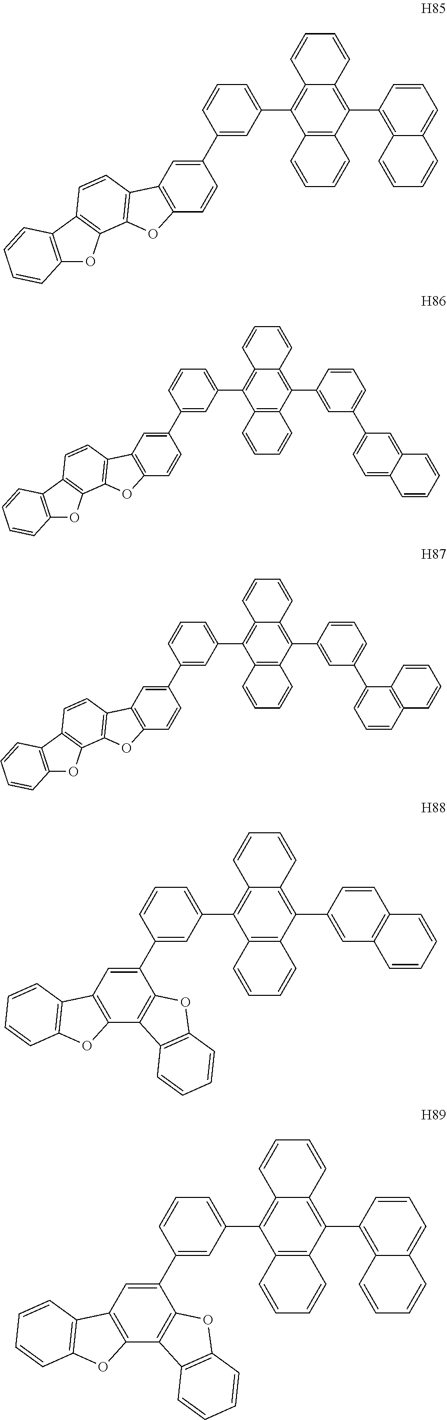 [SMILES: c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1]

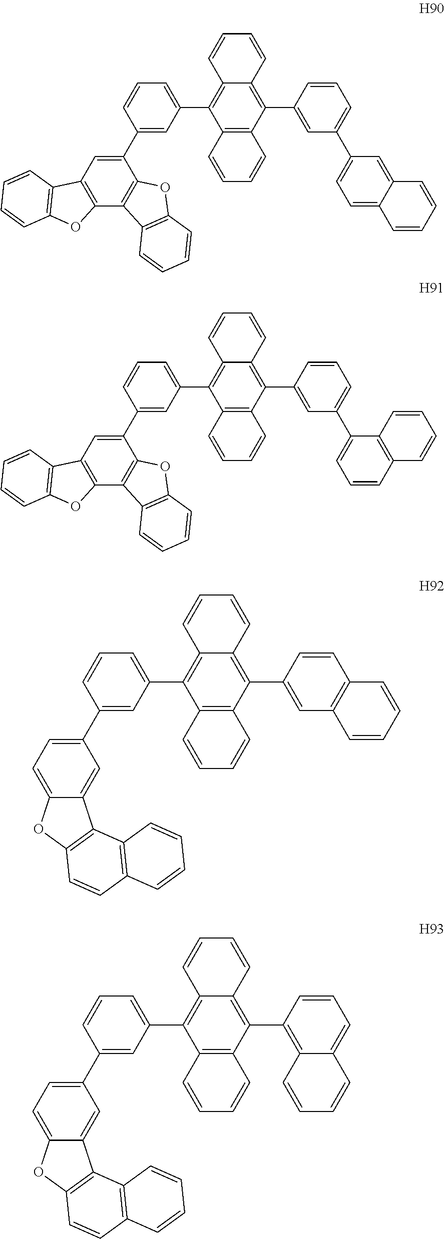 [SMILES: c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1]

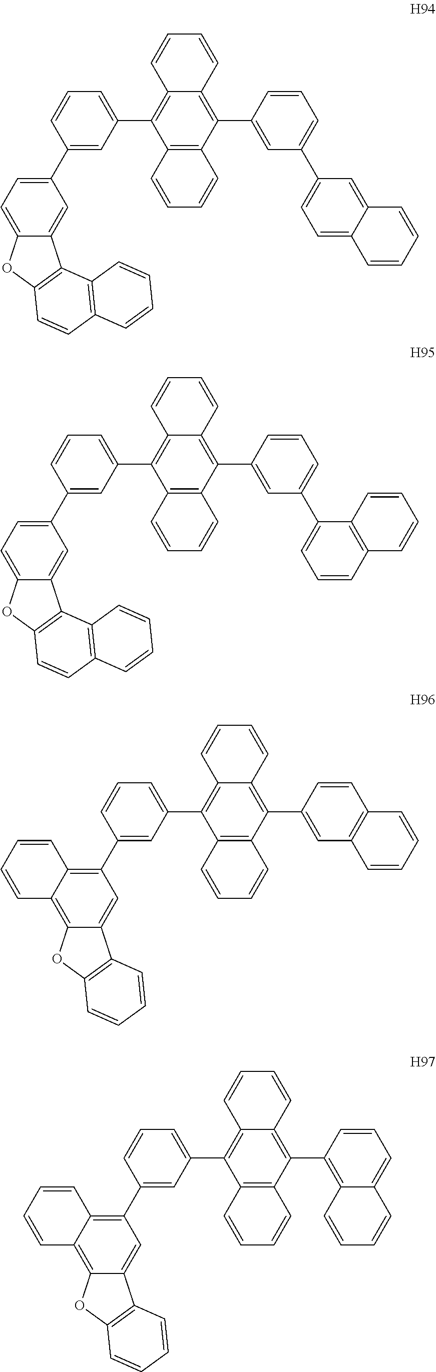 [SMILES: c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1]

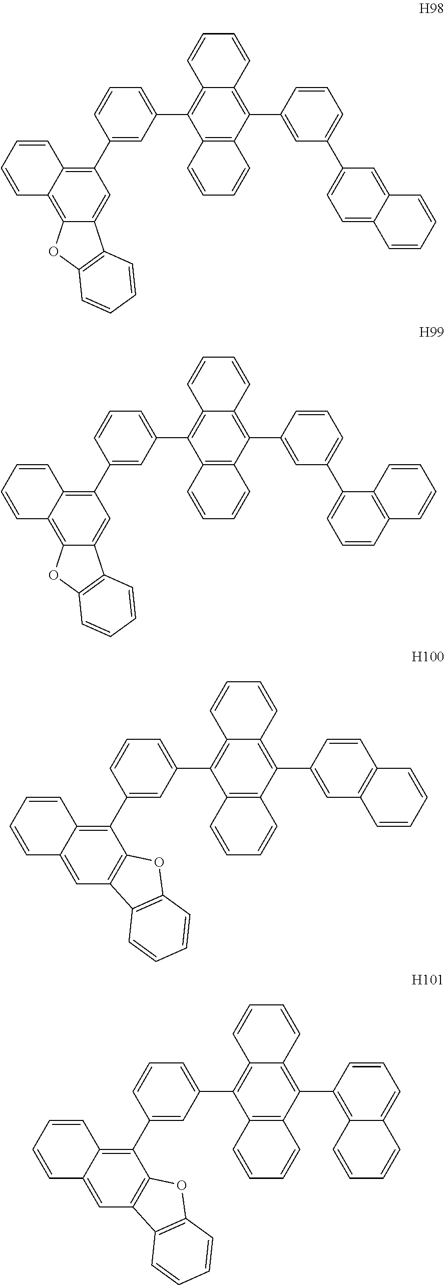 [SMILES: c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1]

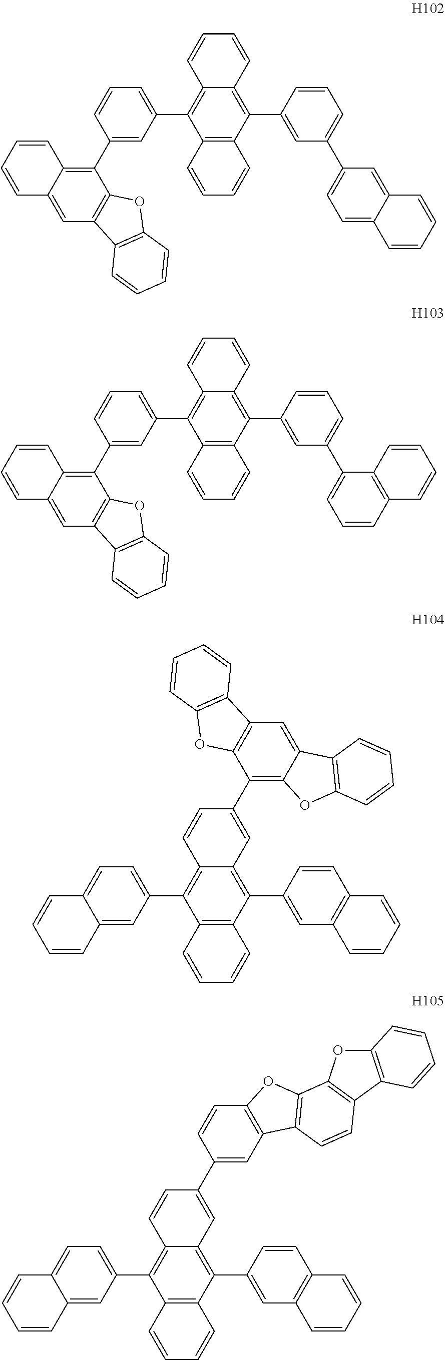 [SMILES: c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1]

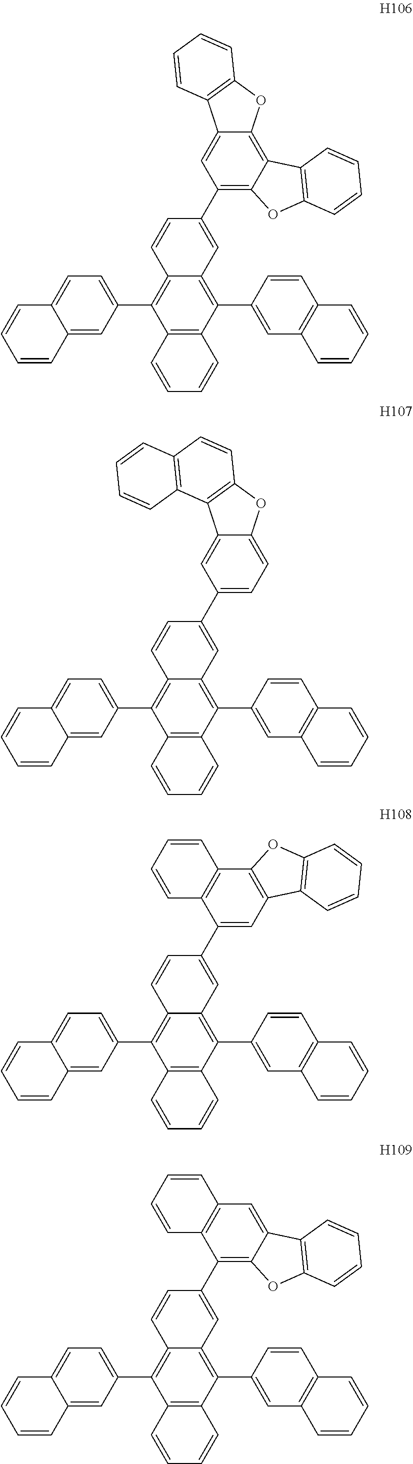

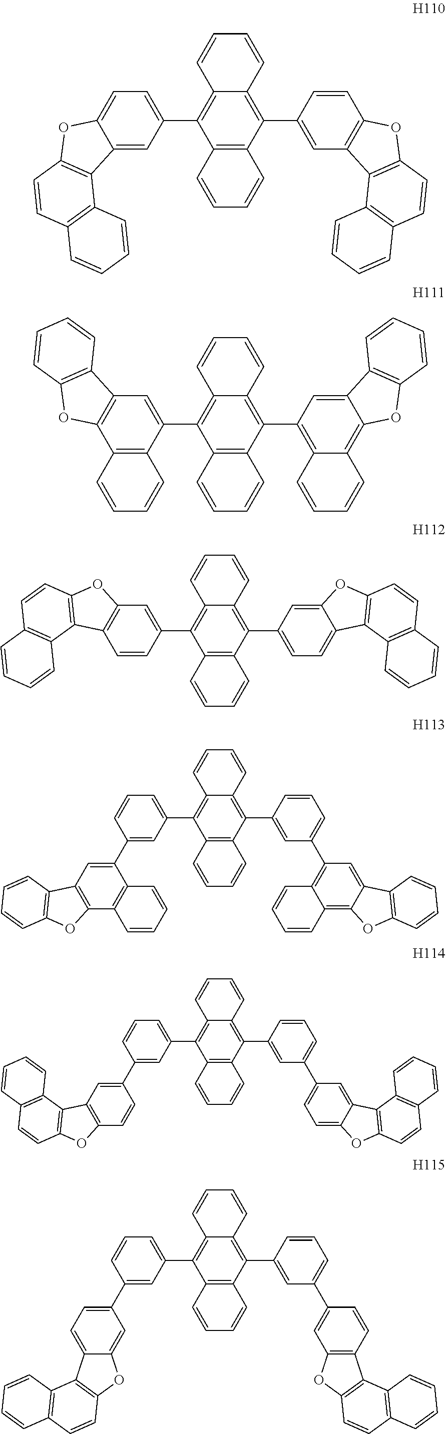

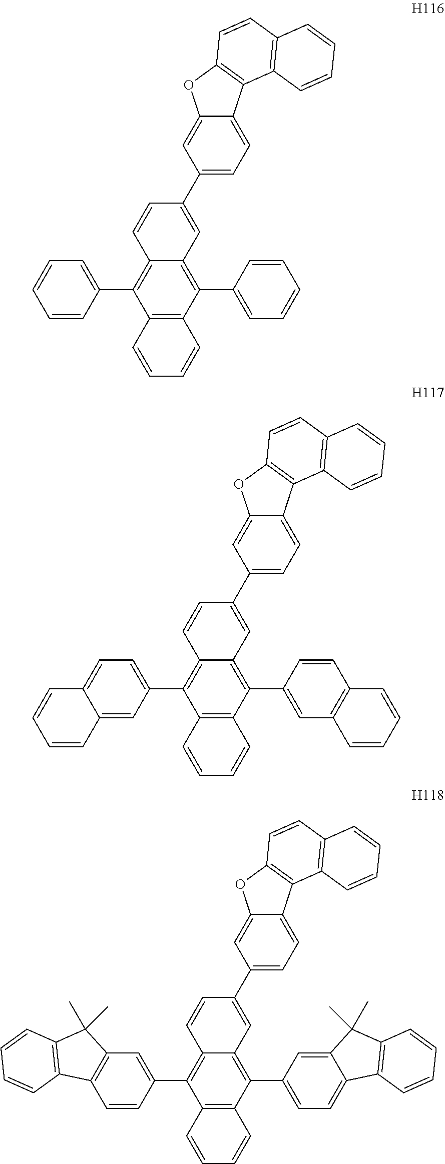 [SMILES: c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

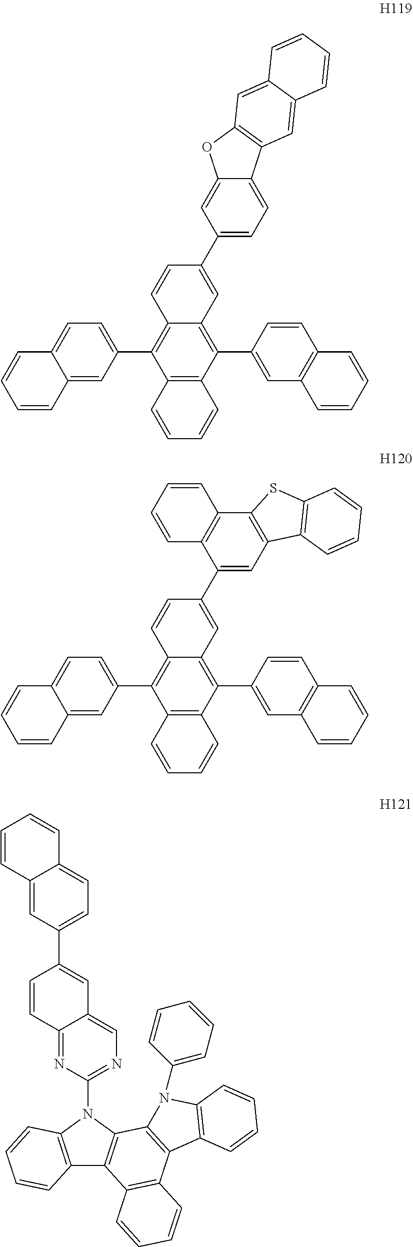

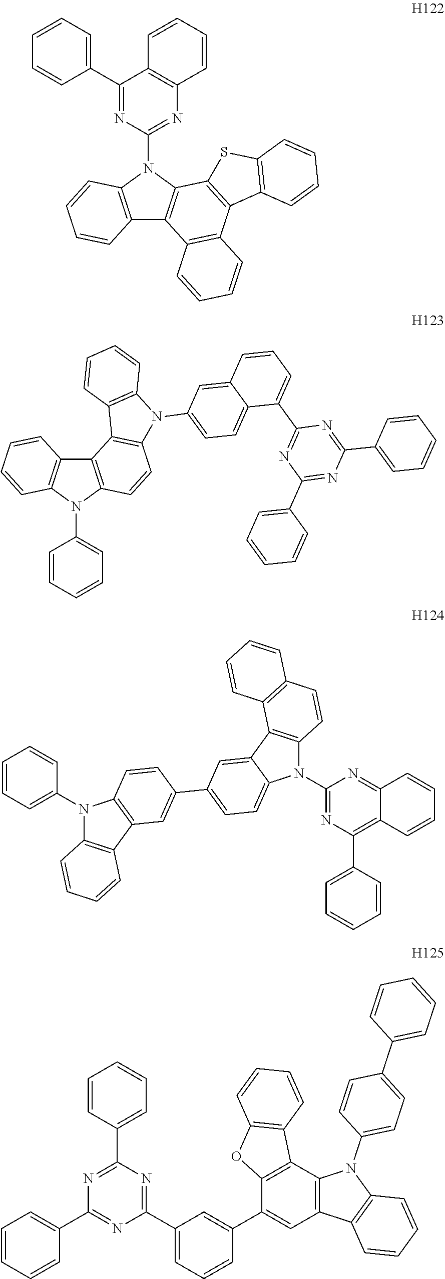 [SMILES: c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

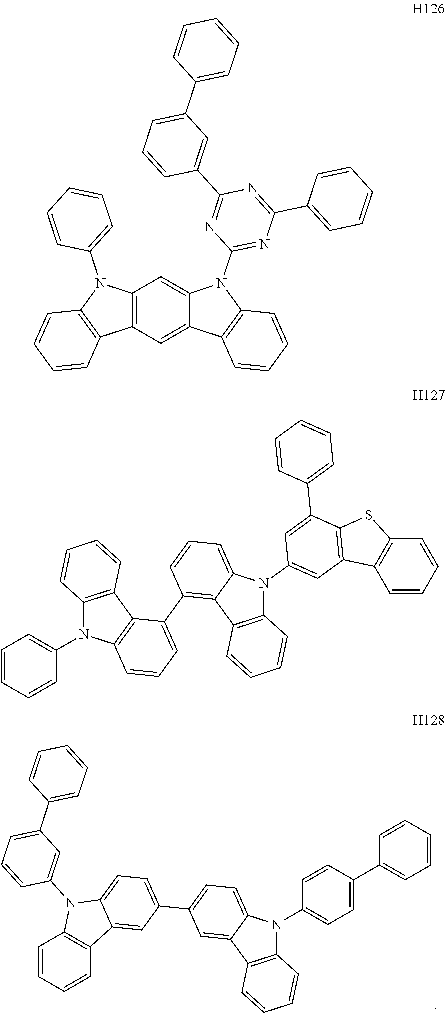 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Electron Transport Region in Interlayer 130

The electron transport region may have i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The electron transport region may include a buffer layer, a hole-blocking layer, an electron control layer, an electron transport layer, an electron injection layer, or any combination thereof.

For example, the electron transport region may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, the layers of each structure being sequentially stacked from the light-receiving layer in the stated order.

In one or more embodiments, the electron transport region (e.g., the buffer layer, the hole-blocking layer, the electron control layer, or the electron transport layer in the electron transport region) may include a metal-free compound including at least one π electron-deficient nitrogen-containing C₁-C₆₀ cyclic group.

For example, in some embodiments, the electron transport region may include a compound represented by Formula 601:

[Ar₆₀₁]ₓₑ₁₁-[(L₆₀₁)ₓₑ₁-R₆₀₁]ₓₑ₂₁, Formula 601
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be the same as described with respect to Q₁,
xe21 may be 1, 2, 3, 4, or 5, and
at least one selected from among Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In one or more embodiments, Ar₆₀₁ in Formula 601 may be a substituted or unsubstituted anthracene group.

In one or more embodiments, the electron transport region may include a compound represented by Formula 601-1:

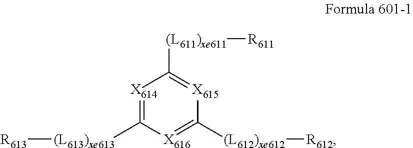

wherein, in Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ may each be the same as described with respect to L₆₀₁,
xe611 to xe613 may each be the same as described with respect to xe1,
R₆₁₁ to R₆₁₃ may each be the same as described with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

In one or more embodiments, the electron transport region may include at least one selected from among Compounds ET1 to ET45, 2,9-dimethyl-4,7-diphenyl-1,10-phenanthroline (BCP), 4,7-diphenyl-1,10-phenanthroline (Bphen), tris(8-hydroxyquinolinato)aluminium (Alq3), bis(2-methyl-8-quinolinolato-N1,O8)-(1,1′-biphenyl-4-olato)aluminum (BAIq), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), or any combination thereof:

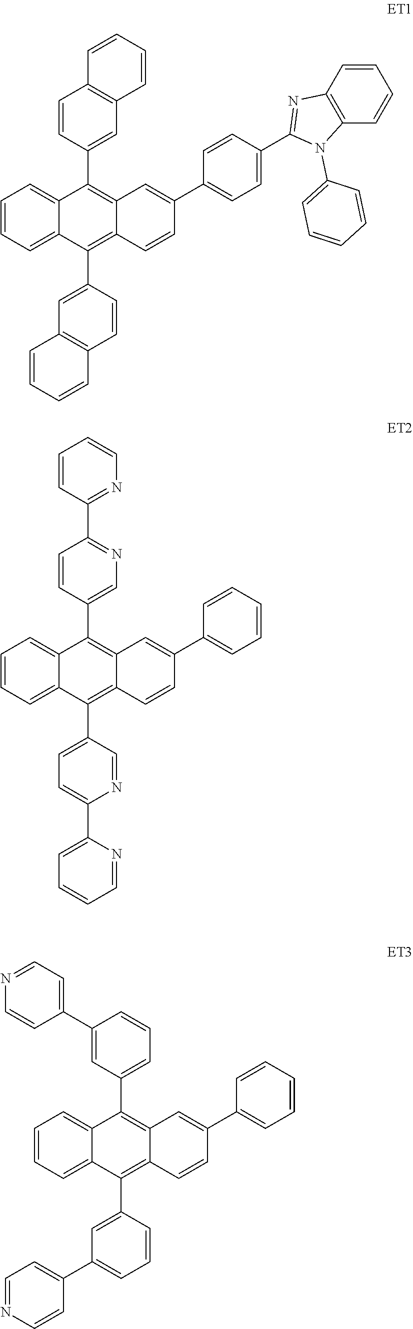 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

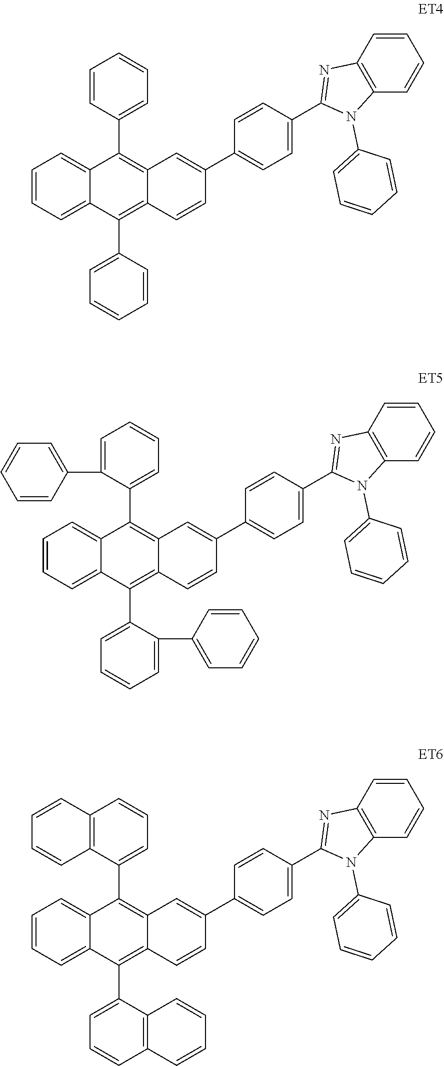 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

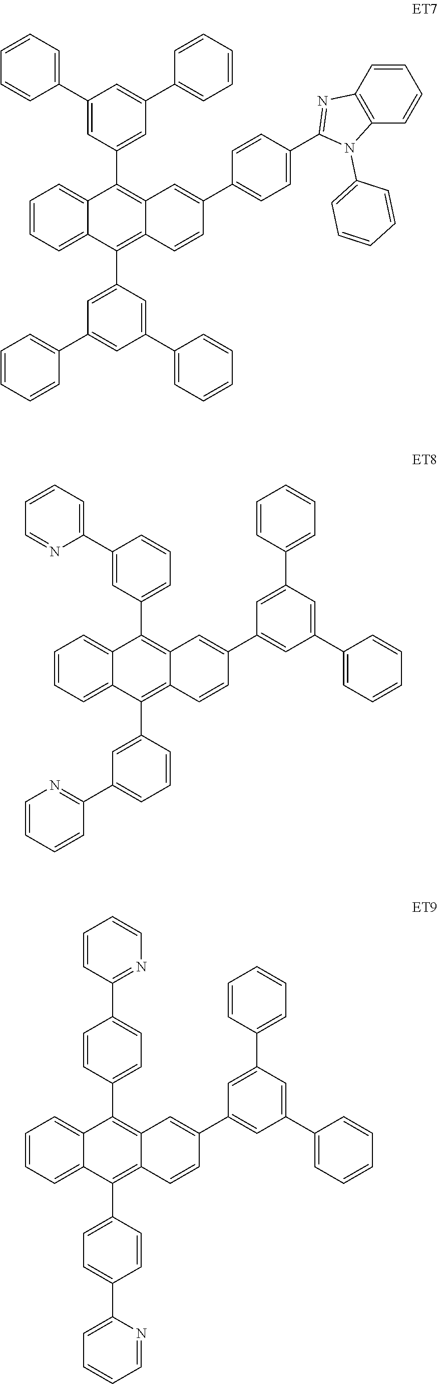 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

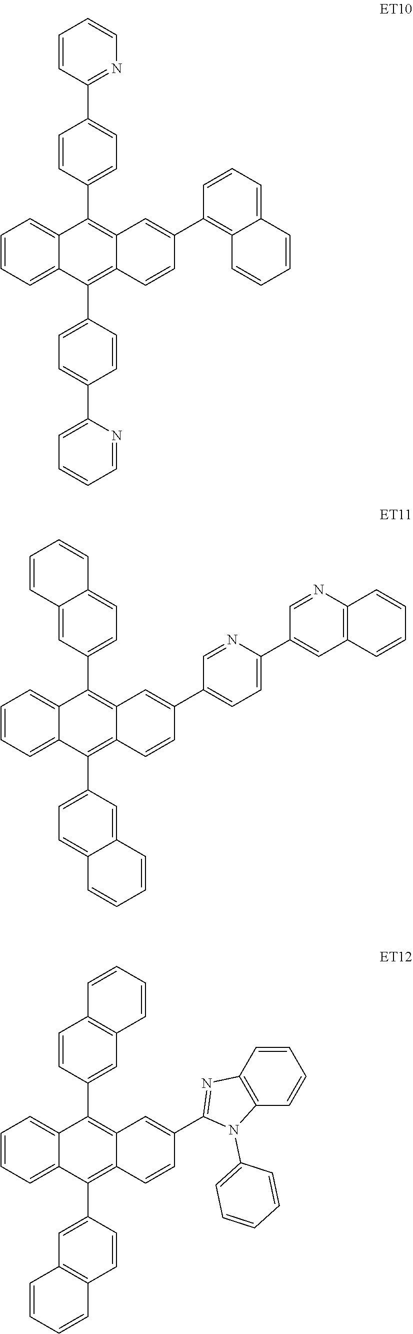 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

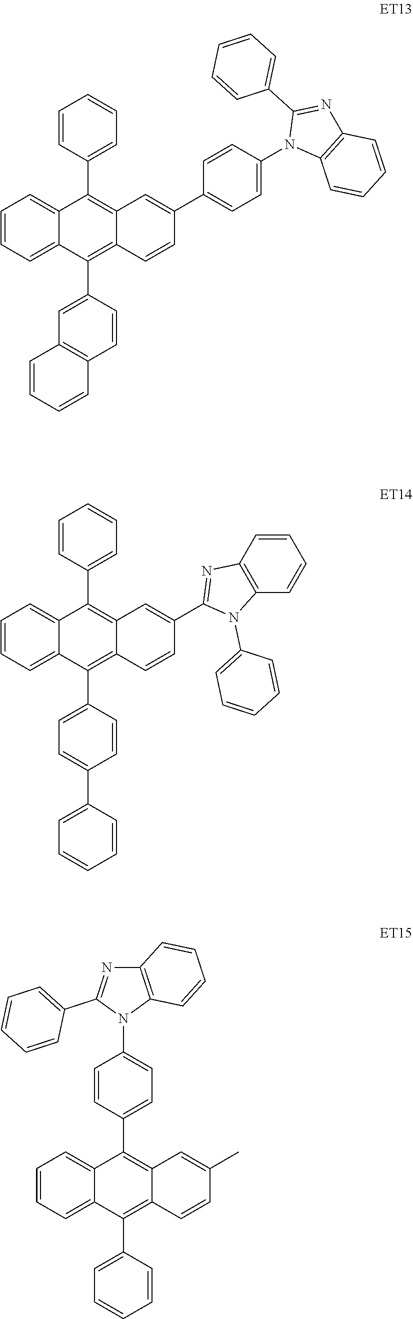 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

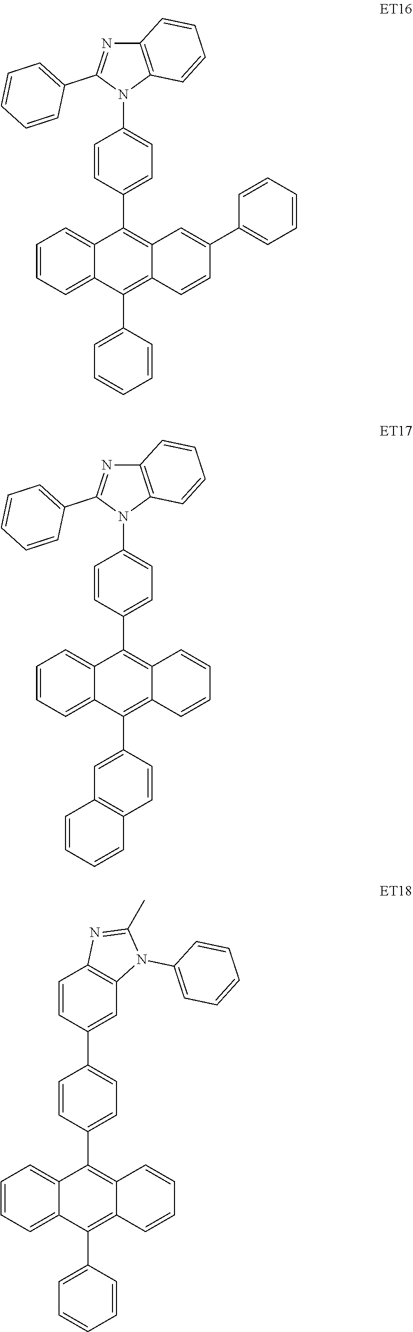 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

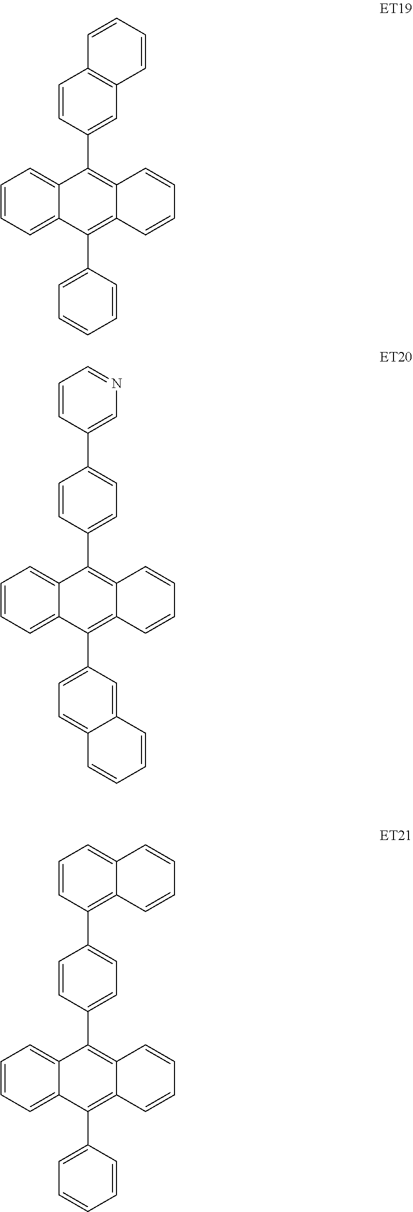

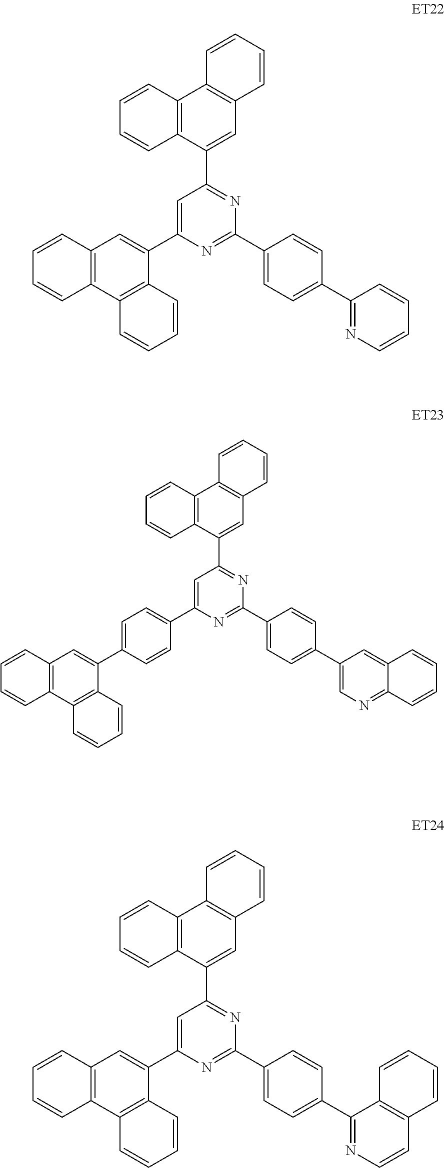 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

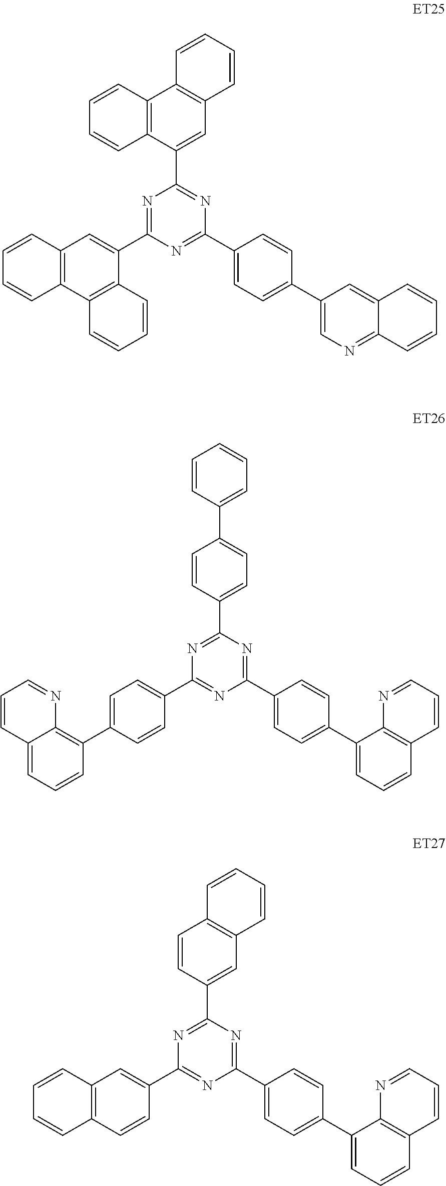 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

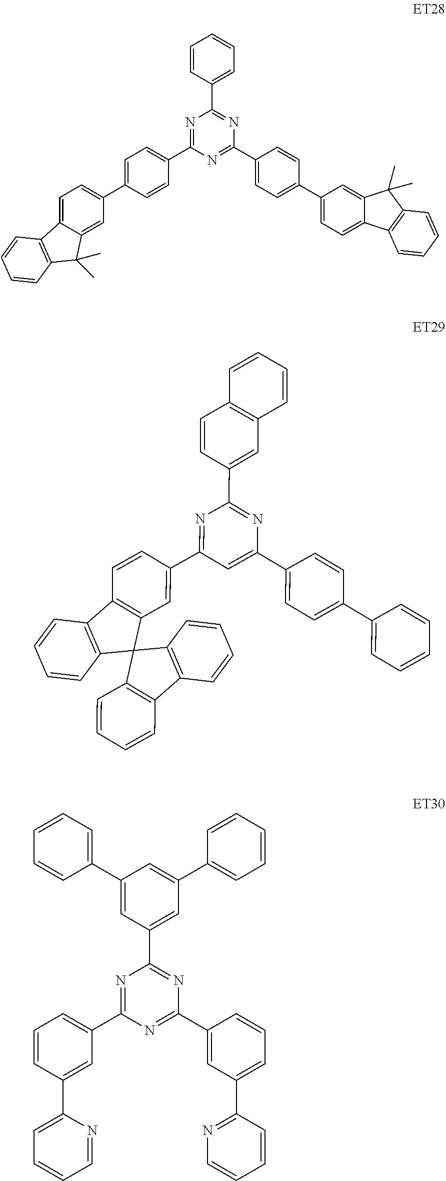 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

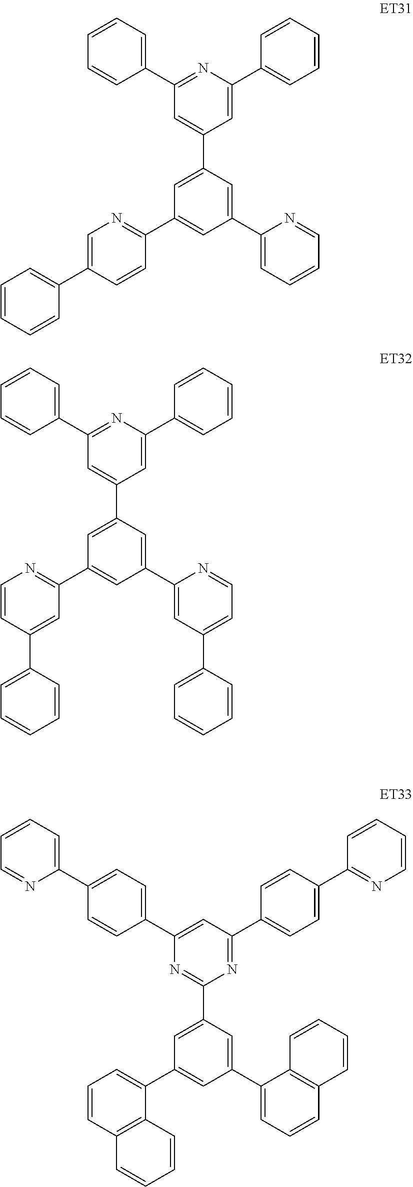

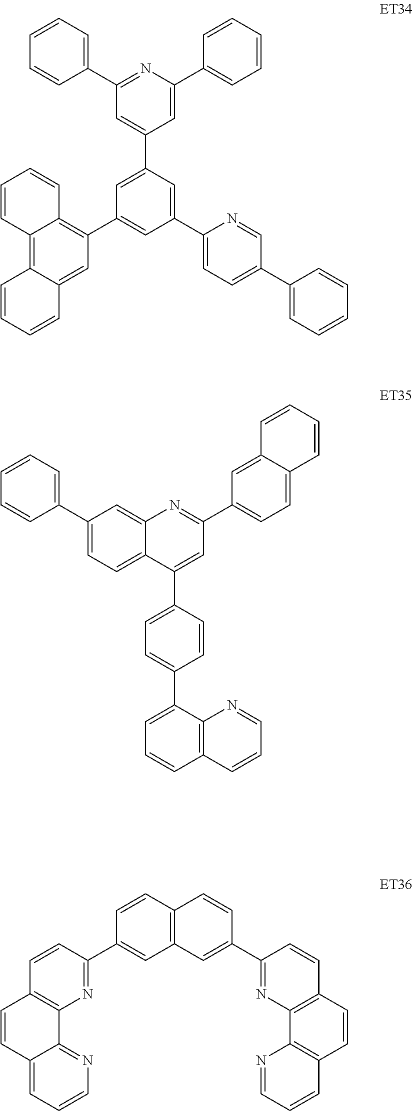 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

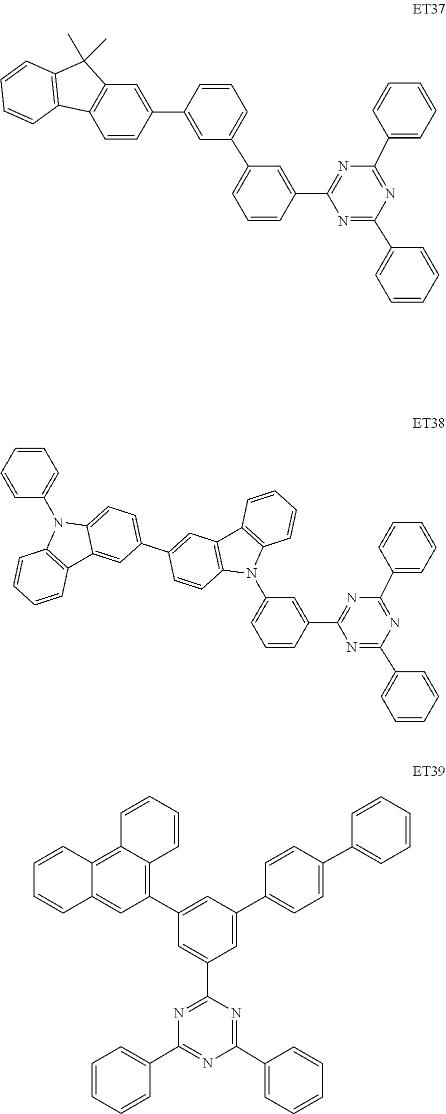 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

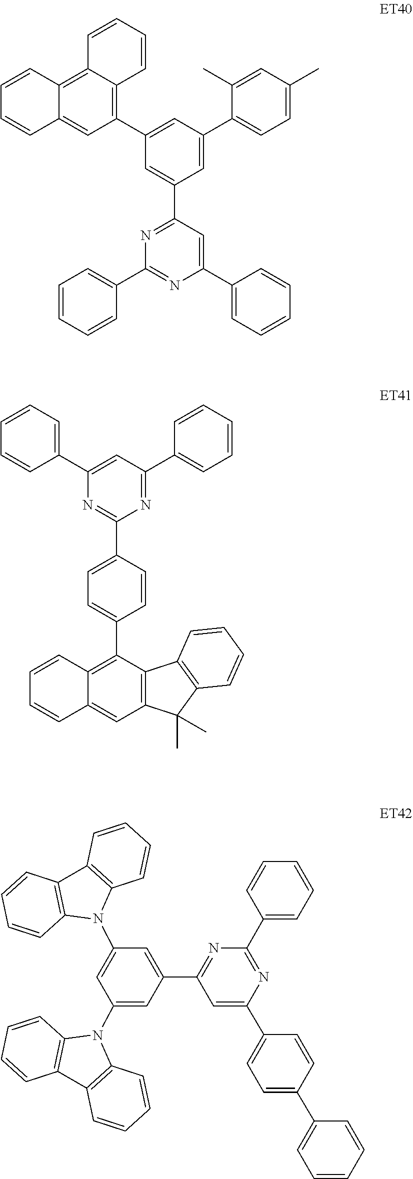 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

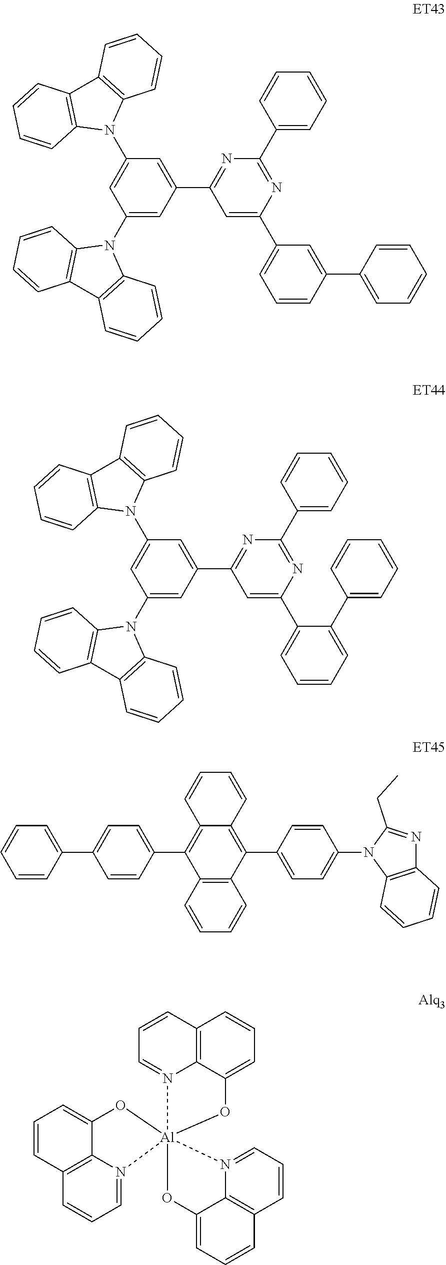

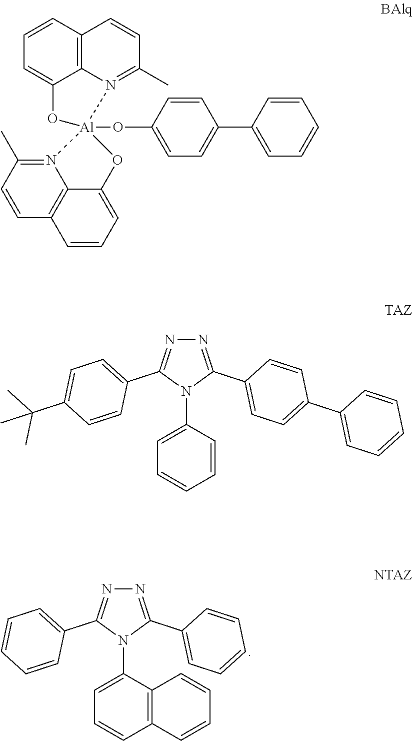

A thickness of the electron transport region may be in a range of about 100 Å to about 5,000 Å, for example, about 160 Å to about 4,000 Å. When the electron transport region includes a buffer layer, a hole-blocking layer, an electron control layer, an electron transport layer, or any combination thereof, a thickness of the buffer layer, the hole-blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole-blocking layer, the electron control layer, the electron transport layer, and/or the electron transport region are within these ranges, satisfactory electron-transporting characteristics may be obtained without a substantial increase in driving voltage.

In one or more embodiments, the electron transport region (e.g., the electron transport layer in the electron transport region) may further include a metal-containing material, in addition to the materials as described above.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, in some embodiments, the metal-containing material may include a Li complex. The Li complex may include, for example, Compound ET-D1 (LiQ) or ET-D2:

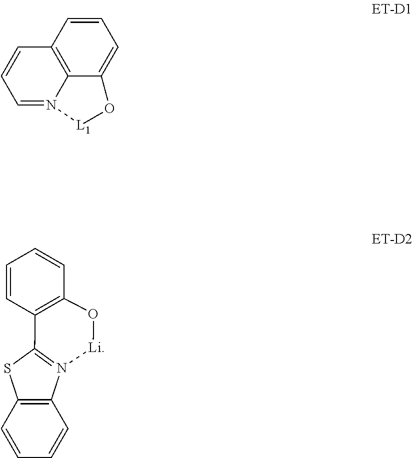

In one or more embodiments, the electron transport region may include an electron injection layer that facilitates the injection of electrons from the second electrode 150. The electron injection layer may be in direct contact with the second electrode 150.

The electron injection layer may have i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof.

The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (e.g., fluorides, chlorides, bromides, iodides, etc.), or tellurides of the alkali metal, the alkaline earth metal, and the rare earth metal, or any combination thereof.

The alkali metal-containing compound may include an alkali metal oxide, such as Li₂O, Cs₂O, or K₂O, an alkali metal halide, such as LiF, NaF, CsF, KF, LiI, NaI, CsI, or KI, or any combination thereof. The alkaline earth metal-containing compound may include an alkaline earth metal compound, such as BaO, SrO, CaO, BaₓSr₁₋ₓO (wherein x is a real number satisfying the condition of 0<x<1), or BaₓCa₁₋ₓO (wherein x is a real number satisfying the condition of 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include a lanthanide metal telluride. Non-limiting examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and/or Lu₂Te₃.

The alkali metal complex, the alkaline earth-metal complex, and the rare earth metal complex may include i) one of ions of the alkali metal, the alkaline earth metal, and the rare earth metal, respectively, and ii) a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (e.g., a compound represented by Formula 601).

In one or more embodiments, the electron injection layer may include (e.g., consist of) i) an alkali metal-containing compound (e.g., an alkali metal halide), or ii) a) an alkali metal-containing compound (e.g., an alkali metal halide); and b) an alkali metal, an alkaline earth metal, a rare earth metal, or any combination thereof. For example, in some embodiments, the electron injection layer may be a KI:Yb co-deposited layer, a RbI:Yb co-deposited layer, a LiF:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within these ranges, satisfactory electron injection characteristics may be obtained without a substantial increase in driving voltage.

### Second Electrode 150

The second electrode 150 may be on the interlayer 130. The second electrode 150 may be a cathode, which is an electron injection electrode, and as the material for the second electrode 150, a metal, an alloy, an electrically conductive compound, or any combination thereof, each having a low work function, may be utilized.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be arranged outside the first electrode 110, and/or a second capping layer may be arranged outside the second electrode 150. In some embodiments, the light-receiving device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the light-receiving layer of the interlayer 130 of the light-receiving device 10 may be extracted toward the outside through the first electrode 110, which is a semi-transmissive electrode or a transmissive electrode, and the first capping layer, or light generated in the light-receiving layer of the interlayer 130 of the light-receiving device 10 may be extracted toward the outside through the second electrode 150, which is a semi-transmissive electrode or a transmissive electrode, and the second capping layer.

The first capping layer and the second capping layer may increase external light-receiving efficiency according to the principle of constructive interference.

Each of the first capping layer and the second capping layer may include a material having a refractive index of 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

In some embodiments, the inorganic capping layer or the organic-inorganic composite capping layer may include the condensed cyclic compound including the first repeating unit and the second repeating unit.

At least one selected from among the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent containing O, N, S, Se, Si, F, Cl, Br, I, or any combination thereof. In one or more embodiments, at least one selected from among the first capping layer and the second capping layer may each independently include an amine group-containing compound.

For example, in some embodiments, at least one selected from among the first capping layer and the second capping layer may each independently include a compound represented by Formula 201, a compound represented by Formula 202, or any combination thereof.

In one or more embodiments, at least one selected from among the first capping layer and the second capping layer may each independently include at least one selected from among Compounds HT28 to HT33, at least one selected from among Compounds CP1 to CP6, β-NPB, and/or any combination thereof:

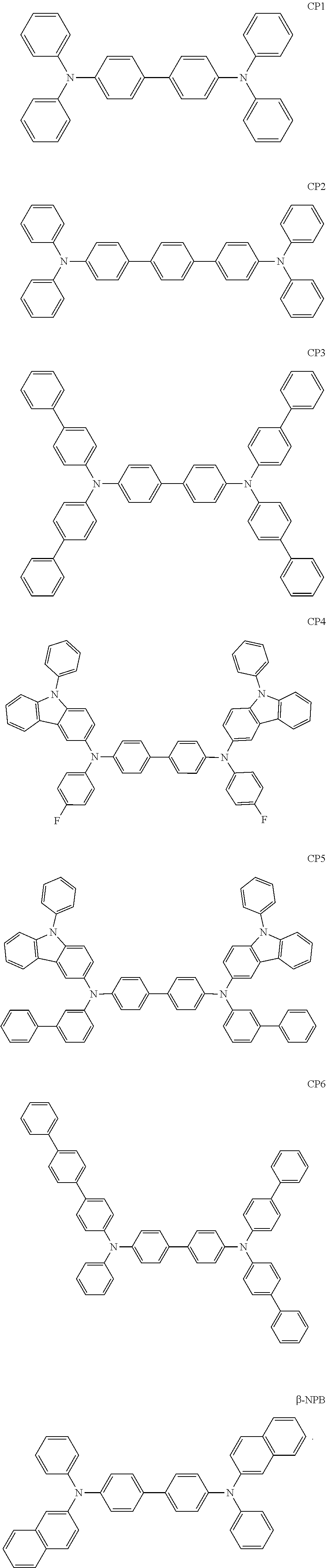

### Film

The condensed cyclic compound of the present disclosure as described herein may be included in one or more suitable films. Accordingly, one or more aspects of embodiments of the present disclosure ae directed toward a film including the condensed cyclic compound. The film may be, for example, an optical member (or a light control means) (e.g., a color filter, a color conversion member, a capping layer, a light-extraction efficiency improvement layer, a selective light-absorbing layer, a polarizing layer, a quantum dot-containing layer, etc.), a light-blocking member (e.g., a light-reflecting layer, a light-absorbing layer, etc.), a protection member (e.g., an insulating layer, a dielectric material layer, etc.), and/or the like.

### Electronic Apparatus

The light-receiving device may be included in one or more suitable electronic apparatuses. For example, in one or more embodiments, the electronic apparatus including the light-receiving device may be an electronic apparatus, an authentication apparatus, and/or the like.

In one or more embodiments, the electronic apparatus may further include, in addition to the light-receiving device, i) a color filter, ii) a color conversion layer, or iii) a color filter and a color conversion layer. The color filter and/or the color conversion layer may be arranged in at least one direction in which light received by the light-receiving device travels. For example, the light received by the light-receiving device may be blue light or white light. The light-receiving device may be the same as described above. In one or more embodiments, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The color filter areas (or the color conversion areas) may include a first area emitting first color light, a second area emitting second color light, and/or a third area emitting third color light, and the first color light, the second color light, and/or the third color light may have different maximum light-receiving wavelengths. For example, the first color light may be red light, the second color light may be green light, and the third color light may be blue light. For example, the color filter areas (or the color conversion areas) may include quantum dots. In detail, the first area may include a red quantum dot, the second area may include a green quantum dot, and the third area may not include (e.g., may exclude) a quantum dot. For details on the quantum dot, related descriptions provided herein may be referred to. The first area, the second area, and/or the third area may each further include a scatterer.

For example, the light-receiving device may be to receive first light, the first area may be to absorb the first light to emit first-first color light, the second area may be to absorb the first light to emit second-first color light, and the third area may be to absorb the first light to emit third-first color light. In this regard, the first-first color light, the second-first color light, and the third-first color light may have different maximum light-receiving wavelengths. In detail, the first light may be blue light, the first-first color light may be red light, the second-first color light may be green light, and the third-first color light may be blue light.

The electronic apparatus may further include a thin-film transistor, in addition to the light-receiving device as described above. The thin-film transistor may include a source electrode, a drain electrode, and an active layer, wherein one of the source electrode or the drain electrode may be electrically connected to the first electrode or the second electrode of the light-receiving device.

The thin-film transistor may further include a gate electrode, a gate insulating film, and/or the like.

The active layer may include crystalline silicon, amorphous silicon, an organic semiconductor, an oxide semiconductor, and/or the like.

The electronic apparatus may further include a sealing portion for sealing the light-receiving device. The sealing portion may be arranged between the color filter and/or the color conversion layer and the light-receiving device. The sealing portion may allow light for the light-receiving device to be absorbed from the outside, and may concurrently (e.g., simultaneously) prevent or reduce ambient air and moisture from penetrating into the light-receiving device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin-film encapsulation layer, the electronic apparatus may be flexible.

Various functional layers may be additionally arranged on the sealing portion, in addition to the color filter and/or the color conversion layer, according to the utilization of the electronic apparatus. Non-limiting examples of the functional layers may include a touch screen layer, a polarizing layer, and/or the like. The touch screen layer may be a pressure-sensitive touch screen layer, a capacitive touch screen layer, or an infrared touch screen layer. The authentication apparatus may be, for example, a biometric authentication apparatus that authenticates an individual by utilizing biometric information of a living body (e.g., fingertips, pupils, etc.).

The authentication apparatus may further include, in addition to the light-receiving device, a biometric information collector.

The electronic apparatus may be applied to one or more suitable displays, light sources, lighting, personal computers (e.g., a mobile personal computer), mobile phones, digital cameras, electronic organizers, electronic dictionaries, electronic game machines, medical instruments (e.g., electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (e.g., meters for a vehicle, an aircraft, and a vessel), projectors, and/or the like.

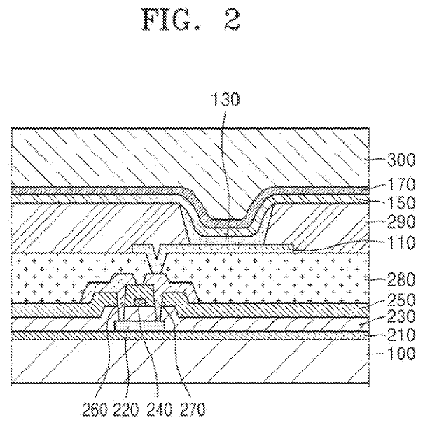

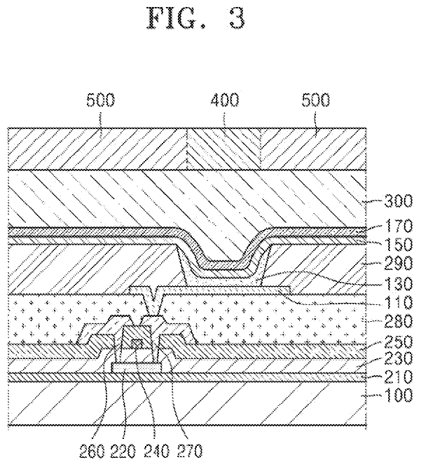

### Descriptions of FIGS. 2 and 3

FIG. 2 is a cross-sectional view of an electronic apparatus according to one or more embodiments of the present disclosure.

The electronic apparatus of FIG. 2 may include a substrate 100, a thin-film transistor (TFT), a light-receiving device, and an encapsulation portion 300 that seals the light-receiving device.

The substrate 100 may be a flexible substrate, a glass substrate, or a metal substrate. A buffer layer 210 may be arranged on the substrate 100. The buffer layer 210 may prevent or reduce penetration of impurities through the substrate 100 and may provide a flat surface on the substrate 100.

A TFT may be arranged on the buffer layer 210. The TFT may include an active layer 220, a gate electrode 240, a source electrode 260, and a drain electrode 270.

The active layer 220 may include an inorganic semiconductor, such as silicon or polysilicon, an organic semiconductor, or an oxide semiconductor, and may include a source region, a drain region, and a channel region.

A gate insulating film 230 for insulating the active layer 220 from the gate electrode 240 may be arranged on the active layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be arranged on the interlayer insulating film 250. The interlayer insulating film 250 and the gate insulating film 230 may be formed to expose the source region and the drain region of the active layer 220, and the source electrode 260 and the drain electrode 270 may be in contact with the exposed portions of the source region and the drain region of the active layer 220, respectively.

The TFT may be electrically connected to a light-receiving device to drive the light-receiving device, and may be covered and protected by a passivation layer 280. The passivation layer 280 may include an inorganic insulating film, an organic insulating film, or any combination thereof. A light-receiving device may be provided on the passivation layer 280. The light-receiving device may include a first electrode 110, an interlayer 130, and a second electrode 150.

The first electrode 110 may be arranged on the passivation layer 280. The passivation layer 280 may not completely cover the drain electrode 270 and may expose a portion of the drain electrode 270, and the first electrode 110 may be connected to the exposed portion of the drain electrode 270.

A pixel-defining layer 290 including an insulating material may be arranged on the first electrode 110. The pixel-defining layer 290 may expose a portion of the first electrode 110, and the interlayer 130 may be formed in the exposed portion of the first electrode 110. The pixel-defining layer 290 may be a polyimide or polyacrylic organic film. In some embodiments, at least some layers of the interlayer 130 may extend beyond the upper portion of the pixel-defining layer 290 to be arranged in the form of a common layer.

The second electrode 150 may be arranged on the interlayer 130, and a capping layer 170 may be additionally formed on the second electrode 150. The capping layer 170 may be formed to cover the second electrode 150.

The encapsulation portion 300 may be arranged on the capping layer 170. The encapsulation portion 300 may be arranged on a light-receiving device to protect the light-receiving device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (e.g., polymethyl methacrylate, polyacrylic acid, etc.), an epoxy-based resin (e.g., aliphatic glycidyl ether (AGE), etc.), or any combination thereof; or a combination of the inorganic film and the organic film.

FIG. 3 is a cross-sectional view of an electronic apparatus according to one or more embodiments of the present disclosure.

The electronic apparatus of FIG. 3 may be substantially the same as the electronic apparatus of FIG. 2, except that a light-shielding pattern 500 and a functional region 400 are additionally arranged on the encapsulation portion 300. The functional region 400 may be i) a color filter area, ii) a color conversion area, iii) a light-guiding area, or iv) a combination of the color filter area, the color conversion area, and the light-guiding area. In one or more embodiments, the light-receiving device included in the electronic apparatus of FIG. 3 may be a tandem light-receiving device.

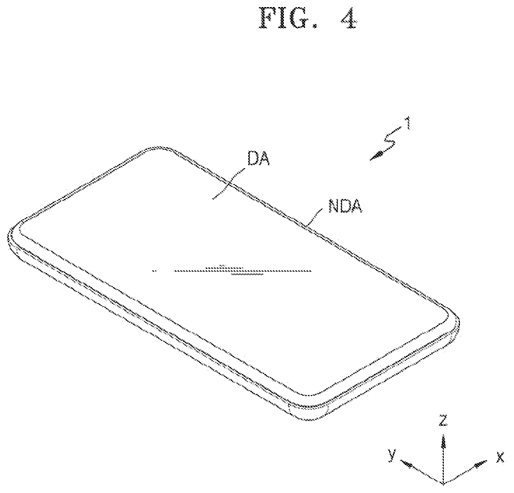

### Description of FIG. 4

FIG. 4 is a schematic perspective view of an electronic device 1 including a light-receiving device, according to one or more embodiments. The electronic device 1 may be a device including an optical sensor, and may include a portable electronic device, such as a mobile phone, a smart phone, a tablet personal computer (PC), a mobile communication terminal, an electronic notebook, an electronic book, a portable multimedia player (PMP), a navigation device, or an ultra mobile PC (UMPC), as well as one or more suitable products, such as a television, a laptop, a monitor, a billboard, or an internet of things (IOT) device, or a part thereof. In one or more embodiments, the electronic device 1 may be a wearable device, such as a smart watch, a watch phone, a glasses-type or kind display, or a head mounted display (HMD), or a part thereof. FIG. 4 shows an embodiment in which the electronic device 1 is a smart phone, for convenience of explanation.

The electronic device 1 may include a display area DA and a non-display area NDA outside the display area DA. A display apparatus of the electronic device 1 may implement an image through an array of a plurality of pixels that are two-dimensionally arranged in the display area DA. The light-receiving device may be to absorb incident light through the display area DA, or may be to absorb incident light through the non-display area NDA.

The non-display area NDA may be an area in which an image is not displayed, and may entirely surround the display area DA. A driver for providing electrical signals or power to display elements arranged in the display area DA may be arranged in the non-display area NDA. A pad, which is an area to which an electronic element or a printed circuit board may be electrically connected, may be arranged in the non-display area NDA.

In the electronic device 1, a length in the x-axis direction and a length in the y-axis direction may be different from each other. For example, as shown in FIG. 4, the length in the x-axis direction may be shorter than the length in the y-axis direction. In one or more embodiments, the length in the x-axis direction may be substantially the same as the length in the y-axis direction. In one or more embodiments, the length in the x-axis direction may be longer than the length in the y-axis direction.

### Manufacturing Method

Respective layers included in the hole transport region, the light-receiving layer, and respective layers included in the electron transport region may be formed in a certain region by utilizing one or more suitable methods, such as vacuum deposition, spin coating, casting, Langmuir-Blodgett (LB) deposition, ink-jet printing, laser-printing, and/or laser-induced thermal imaging (LITI).

When layers constituting the hole transport region, the light-receiving layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as utilized herein refers to a cyclic group including (e.g., consisting of) carbon only as a ring-forming atom and having 3 to 60 carbon atoms, and the term “C₁-C₆₀ heterocyclic group” as utilized herein refers to a cyclic group that has 1 to 60 carbon atoms and further has, in addition to carbon, a heteroatom as a ring-forming atom. The C₃-C₆₀ carbocyclic group and the C₁-C₆₀ heterocyclic group may each be a monocyclic group including (e.g., consisting of) one ring or a polycyclic group in which two or more rings are condensed with each other. For example, the C₁-C₆₀ heterocyclic group may have 3 to 61 ring-forming atoms.

The term “cyclic group” as utilized herein may include the C₃-C₆₀ carbocyclic group and the C₁-C₆₀ heterocyclic group.

The term “π electron-rich C₃-C₆₀ cyclic group” as utilized herein refers to a cyclic group that has 3 to 60 carbon atoms and does not include *—N═*′ as a ring-forming moiety, and the term “π electron-deficient nitrogen-containing C₁-C₆₀ cyclic group” as utilized herein refers to a heterocyclic group that has 1 to 60 carbon atoms and includes *—N═*′ as a ring-forming moiety.

For example,
the C₃-C₆₀ carbocyclic group may be i) a group T1 or ii) a condensed cyclic group in which two or more groups T1 are condensed with each other (e.g., a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a group T2, ii) a condensed cyclic group in which two or more groups T2 are condensed with each other, or iii) a condensed cyclic group in which at least one group T2 and at least one group T1 are condensed with each other (e.g., a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) a group T1, ii) a condensed cyclic group in which two or more groups T1 are condensed with each other, iii) a group T3, iv) a condensed cyclic group in which two or more groups T3 are condensed with each other, or v) a condensed cyclic group in which at least one group T3 and at least one group T1 are condensed with each other (e.g., the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.), and
the π electron-deficient nitrogen-containing C₁-C₆₀ cyclic group may be i) a group T4, ii) a condensed cyclic group in which two or more group T4 are condensed with each other, iii) a condensed cyclic group in which at least one group T4 and at least one group T1 are condensed with each other, iv) a condensed cyclic group in which at least one group T4 and at least one group T3 are condensed with each other, or v) a condensed cyclic group in which at least one group T4, at least one group T1, and at least one group T3 are condensed with one another (e.g., a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
wherein the group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,
the group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the group T3 may be a furan group, a thiophene group, a 1H-pyrrole group, a silole group, or a borole group, and
the group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as utilized herein refer to a group condensed to any cyclic group, a monovalent group, or a polyvalent group (e.g., a divalent group, a trivalent group, a tetravalent group, etc.) according to the structure of a formula for which the corresponding term is utilized. For example, the “benzene group” may be a benzo group, a phenyl group, a phenylene group, and/or the like, which may be easily understood by one of ordinary skill in the art according to the structure of a formula including the “benzene group.”

Non-limiting examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Non-limiting examples of the divalent C₃-C₆₀ carbocyclic group and the divalent C₁-C₆₀ heterocyclic group may include a C₃-C₁ cycloalkylene group, a C₁-C₁₀ heterocycloalkylene group, a C₃-C₁ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as utilized herein refers to a linear or branched aliphatic hydrocarbon monovalent group that has 1 to 60 carbon atoms, and non-limiting examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as utilized herein refers to a divalent group having the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of the C₂-C₆₀ alkyl group, and non-limiting examples thereof may include an ethenyl group, a propenyl group, and a butenyl group. The term “C₂-C₆₀ alkenylene group” as utilized herein refers to a divalent group having the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of the C₂-C₆₀ alkyl group, and non-limiting examples thereof may include an ethynyl group and a propynyl group. The term “C₂-C₆₀ alkynylene group” as utilized herein refers to a divalent group having the same structure as the C₂-C₆₀ alkynyl group.

The term “C₁-C₆₀ alkoxy group” as utilized herein refers to a monovalent group represented by —OA₁₀₁ (wherein A₁₀₁ is the C₁-C₆₀ alkyl group), and non-limiting examples thereof may include a methoxy group, an ethoxy group, and an isopropyloxy group.

The term “C₃-C₁₀ cycloalkyl group” as utilized herein refers to a monovalent saturated hydrocarbon cyclic group having 3 to 10 carbon atoms, and non-limiting examples thereof may include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as utilized herein refers to a divalent group having the same structure as the C₃-C₁₀ cycloalkyl group.

The term “C₁-C₁ heterocycloalkyl group” as utilized herein refers to a monovalent cyclic group that further includes, in addition to a carbon atom, at least one heteroatom as a ring-forming atom and has 1 to 10 carbon atoms, and non-limiting examples thereof may include a 1,2,3,4-oxatriazolidinyl group, a tetrahydrofuranyl group, and a tetrahydrothiophenyl group. The term “C₁-C₁ heterocycloalkylene group” as utilized herein refers to a divalent group having the same structure as the C₁-C₁₀ heterocycloalkyl group.

The term “C₃-C₁₀ cycloalkenyl group” utilized herein refers to a monovalent cyclic group that has 3 to 10 carbon atoms and at least one carbon-carbon double bond in the ring thereof and no aromaticity, and non-limiting examples thereof may include a cyclopentenyl group, a cyclohexenyl group, and a cycloheptenyl group. The term “C₃-C₁₀ cycloalkenylene group” as utilized herein refers to a divalent group having the same structure as the C₃-C₁₀ cycloalkenyl group.

The term “C₁-C₁₀ heterocycloalkenyl group” as utilized herein refers to a monovalent cyclic group of 1 to 10 carbon atoms that further includes, in addition to carbon atoms, at least one heteroatom as a ring-forming atom and has at least one double bond in its ring. Non-limiting examples of the C₁-C₁₀ heterocycloalkenyl group may include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as utilized herein refers to a divalent group having the same structure as the C₁-C₁₀ heterocycloalkenyl group.

The term “C₆-C₆₀ aryl group” as utilized herein refers to a monovalent group having a carbocyclic aromatic system of 6 to 60 carbon atoms, and the term “C₆-C₆a arylene group” as utilized herein refers to a divalent group having a carbocyclic aromatic system of 6 to 60 carbon atoms. Non-limiting examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the two or more rings may be condensed with each other.

The term “C₁-C₆₀ heteroaryl group” as utilized herein refers to a monovalent group having a heterocyclic aromatic system that has, in addition to a carbon atom, at least one heteroatom as a ring-forming atom, and 1 to 60 carbon atoms, and the term “C₁-C₆₀ heteroarylene group” as utilized herein refers to a divalent group having a heterocyclic aromatic system that has, in addition to a carbon atom, at least one heteroatom as a ring-forming atom, and 1 to 60 carbon atoms. Non-limiting examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the two or more rings may be condensed with each other.

The term “monovalent non-aromatic condensed polycyclic group” as utilized herein refers to a monovalent group (e.g., having 8 to 60 carbon atoms) having two or more rings condensed with each other, only carbon atoms as ring-forming atoms, and no aromaticity in its entire molecular structure as a whole. Non-limiting examples of the monovalent non-aromatic condensed polycyclic group may include an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as utilized herein refers to a divalent group having the same structure as the monovalent non-aromatic condensed polycyclic group.

The term “monovalent non-aromatic condensed heteropolycyclic group” as utilized herein refers to a monovalent group (e.g., having 1 to 60 carbon atoms) having two or more rings condensed with each other, at least one heteroatom other than carbon atoms, as a ring-forming atom, and no aromaticity in its entire molecular structure as a whole. Non-limiting examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as utilized herein refers to a divalent group having the same structure as the monovalent non-aromatic condensed heteropolycyclic group.

The term “C₆-C₆₀ aryloxy group” as utilized herein refers to —OA₁₀₂ (wherein A₁₀₂ is the C₆-C₆₀ aryl group), and the term “C₆-C₆₀ arylthio group” as utilized herein refers to —SA₁₀₃ (wherein A₁₀₃ is the C₆-C₆₀ aryl group).

The term “C₇-C₆₀ arylalkyl group” as utilized herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as utilized herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as utilized herein refers to:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ as utilized herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as utilized herein refers to any atom other than a carbon atom. Non-limiting examples of the heteroatom may include O, S, N, P, Si, B, Ge, Se, or any combination thereof.

The term “third-row transition metal” utilized herein includes hafnium (Hf), tantalum (Ta), tungsten (W), rhenium (Re), osmium (Os), iridium (Ir), platinum (Pt), gold (Au), and/or the like.

“Ph” as utilized herein refers to a phenyl group, “Me” as utilized herein refers to a methyl group, “Et” as utilized herein refers to an ethyl group, “tert-Bu” or “Bu^{t}” as utilized herein refers to a tert-butyl group, and “OMe” as utilized herein refers to a methoxy group.

The term “biphenyl group” as utilized herein refers to “a phenyl group substituted with a phenyl group.” In other words, the “biphenyl group” is a substituted phenyl group having a C₆-C₆₀ aryl group as a substituent.

The term “terphenyl group” as utilized herein refers to “a phenyl group substituted with a biphenyl group.” In other words, the “terphenyl group” is a substituted phenyl group having, as a substituent, a C₆-C₆₀ aryl group substituted with a C₆-C₆₀ aryl group.

* and *′ as utilized herein, unless defined otherwise, each refer to a binding site to a neighboring atom in a corresponding formula or moiety.

The x-axis, y-axis, and z-axis as utilized herein are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, compounds according to embodiments and light-receiving devices according to embodiments will be described in more detail with reference to Synthesis Examples and Examples. The wording “B was utilized instead of A” utilized in describing Synthesis Examples refers to that an identical molar equivalent of B was utilized in place of A.

### Examples

### Synthesis Example 1: Synthesis of Compound 1

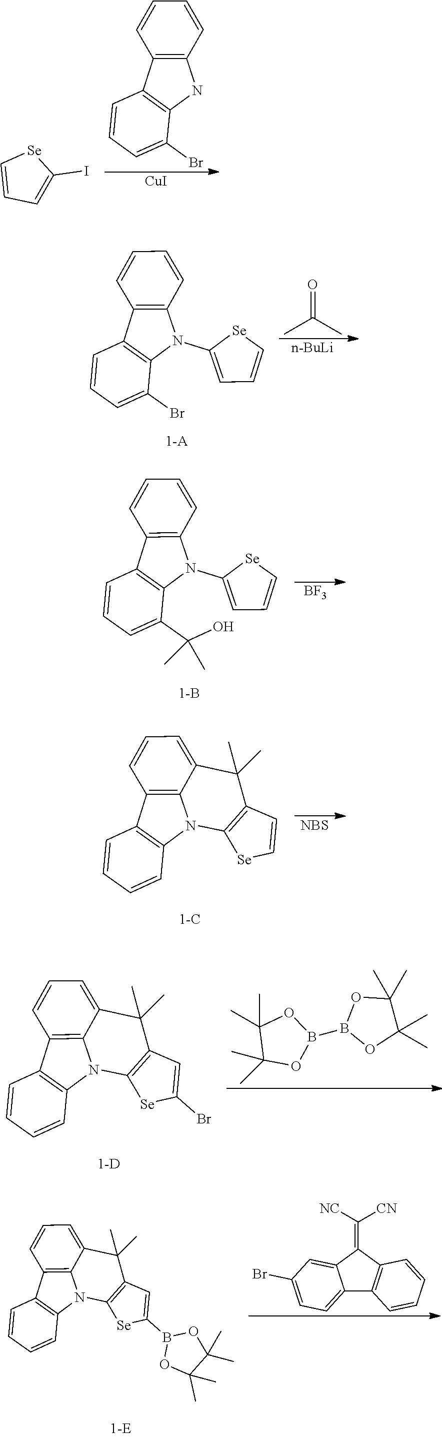

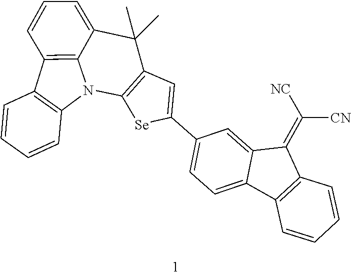 [SMILES: CC1(C)c2cc(-c3ccc4c(c3)C(=C(C#N)C#N)c3ccccc3-4)[se]c2-n2c3ccccc3c3cccc1c32]

### Synthesis of Intermediate 1-A

10 g (38.9 mmol) of 2-iodoselenophene and 7.8 g (32 mmol) of 1-bromo-9H-carbazole were dissolved in 40 mL of dioxane. 0.35 g (1.8 mmol) of copper (I) iodide, 0.70 g (6.09 mmol) of trans-1,2-cyclohexanediamine, and 12.9 g (61.0 mmol) of tripotassium phosphate were added thereto, and the resultant reaction solution was refluxed while heating for 30 hours. The resultant product was separated and purified by silica gel column chromatography (volume ratio of hexane:ethyl acetate as an eluent=5:1), and 8.4 g (yield: 70%) of Intermediate 1-A was obtained. The obtained compound was identified by MS/FAB.

C₁₆H₁₀BrNSe: calc. 375.14, found 375.18 .

### Synthesis of Intermediate I-B

8.4 g (22.0 mmol) of Intermediate 1-A was dissolved in 250 mL of dehydrated diethyl ether. 8 mL (32.0 mmol) of a hexane solution including 2.76 M n-butyl lithium (n-BuLi) was added dropwise thereto at −50° C., and the resultant reaction solution was stirred at room temperature for 1 hour. 1.3 g (25 mmol) of dehydrated acetone (or dimethyl ketone) (CH₃COCH₃) was added thereto at −50° C., and the resultant reaction solution was stirred at room temperature for 2 hours. An organic layer extracted therefrom utilizing dehydrated diethyl ether was washed with an aqueous sodium chloride solution and dried with anhydrous magnesium sulfate. The resultant product was separated and purified by silica gel column chromatography (performed while changing the volume ratio of solvent, hexane:dichloromethane as an eluent=100:0 to 50:50), and 5.1 g (yield: 66%) of Intermediate 1-B was obtained. The obtained compound was identified by MS/FAB.

C₁₆H₁₇NOSe: calc. 354.32, found 355.18 .

### Synthesis of Intermediate 1-C

5.1 g (14.3 mmol) of Intermediate 1-B was dissolved in 180 mL of dichloromethane. 4.98 g (35.5 mmol) of a boron trifluoride-ethyl ether complex was added dropwise thereto at 0° C., and the resultant reaction solution was stirred for 2 hours. An organic layer extracted from dichloromethane was washed with an aqueous sodium chloride solution and dried with anhydrous magnesium sulfate. The resultant product was separated and purified by silica gel column chromatography (volume ratio of hexane:dichloromethane as an eluent=50:50), and 4.09 g (yield: 85%) of Intermediate 1-C was obtained. The obtained compound was identified by MS/FAB.

C₁₉H15NSe: calc. 336.31, found 336.42 .

### Synthesis of Intermediate 1-D

4.09 g (12.1 mmol) of Intermediate 1-C, 3.76 g (13.3 mmol) of 4-bromoiodobenzene, 1.16 g (1.00 mmol) of tetrakis(triphenylphosphine)palladium (Pd(PPh₃)₄), and 2.48 g (17.92 mmol) of K₂CO₃ were dissolved in 120 mL of a mixed solution of THF/H₂O (volume ratio of 2:1), and the resultant reaction solution was stirred at 70° C. for 5 hours. The reaction solution was cooled to room temperature, 40 mL of water was added to thereto, and an organic layer was extracted therefrom three times utilizing 50 mL of ethyl ether. The collected organic layer was dried with magnesium sulfate, the residue obtained by evaporating the solvent therefrom was separated and purified by silica gel column chromatography, and 3.72 g (yield: 74%) of Intermediate 1-D was obtained. The obtained compound was identified by MS/FAB.

C₁₉H₁₄BrNSe: calc. 415.20, found 415.39 .

### Synthesis of Intermediate 1-E

3.72 g (8.96 mmol) of Intermediate 1-D, 2.28 g (8.96 mmol) of bis(pinacolato)diboron, 0.36 g (0.5 mmol) of PdCl₂(dppf)₂, and 2.94 g (30.0 mmol) of KOAc were dissolved in 40 mL of DMSO, and the resultant reaction solution was stirred at 80° C. for 6 hours. The reaction solution was cooled to room temperature, and an organic layer was extracted therefrom three times utilizing 50 mL of water and 50 mL of diethyl ether. The obtained organic layer was dried with magnesium sulfate, the residue obtained by evaporating the solvent therefrom was separated and purified by silica gel column chromatography, and 3.3 g (yield: 80%) of Intermediate 1-E was obtained. The obtained compound was identified by MS/FAB.

C₂₅H₂₆BNO₂Se: calc. 462.27, found 462.35 .

### Synthesis of Compound 1

3.3 g (7.14 mmol) of Intermediate 1-E, 2.19 g (7.14 mmol) of 2-(2-bromo-9H-fluoren-9-ylidene)propanedinitrile, 116 g (1.00 mmol) of Pd(PPh₃)₄, and 2.48 g (17.92 mmol) of K₂CO₃ were dissolved in 120 mL of a mixed solution of THF/H₂O (volume ratio of 2:1), and the resultant reaction solution was stirred at 70° C. for 5 hours. The reaction solution was cooled to room temperature, 60 mL of water was added to thereto, and an organic layer was extracted therefrom three times utilizing 80 mL of ethyl ether. The collected organic layer was dried with magnesium sulfate, the residue obtained by evaporating the solvent therefrom was separated and purified by silica gel column chromatography, and 2.97 g (yield: 74%) of Compound 1 was obtained. The obtained compound was identified by ¹H NMR (CDCl₃, 400 MHz) and MS/FAB.

¹H NMR: δ=8.57-8.55 (m, 1H), 8.29-8.11 (m, 3H), 7.71-7.67 (m, 2H), 7.53-7.41 (m, 4H), 7.36-7.08 (m, 4H), 6.86 (s, 1H), 1.46 (s, 6H)

C₃₅H₂₁N₃Se: calc. 562.55, found 562.75 .

### Synthesis Example 2: Synthesis of Compound 3

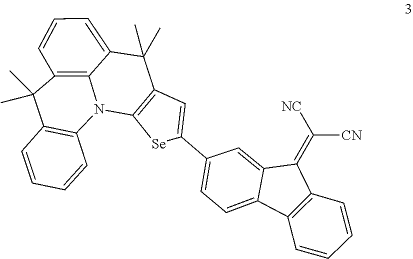 [SMILES: CC1(C)c2ccccc2N2c3[se]c(-c4ccc5c(c4)C(=C(C#N)C#N)c4ccccc4-5)cc3C(C)(C)c3cccc1c32]

Compound 3 was synthesized in substantially the same manner as utilized to synthesize Compound 1, except that 4-bromo-9H-acridine was utilized instead of 1-bromo-9H-carbazole during the synthesis of Intermediate 1-A in Synthesis Example 1. The obtained compound was identified by ¹H NMR (CDCl₃, 400 MHz) and MS/FAB.

¹H NMR: δ=8.29-8.25 (m, 1H), 7.53-7.41 (m, 5H), 7.37-7.14 (m, 7H), 6.95-6.86 (m, 2H), 1.69 (s, 6H), 1.46 (s, 6H)

C₃₈H₂₇N₃Se: calc. 604.63, found 604.75 .

### Synthesis Example 3: Synthesis of Compound 5

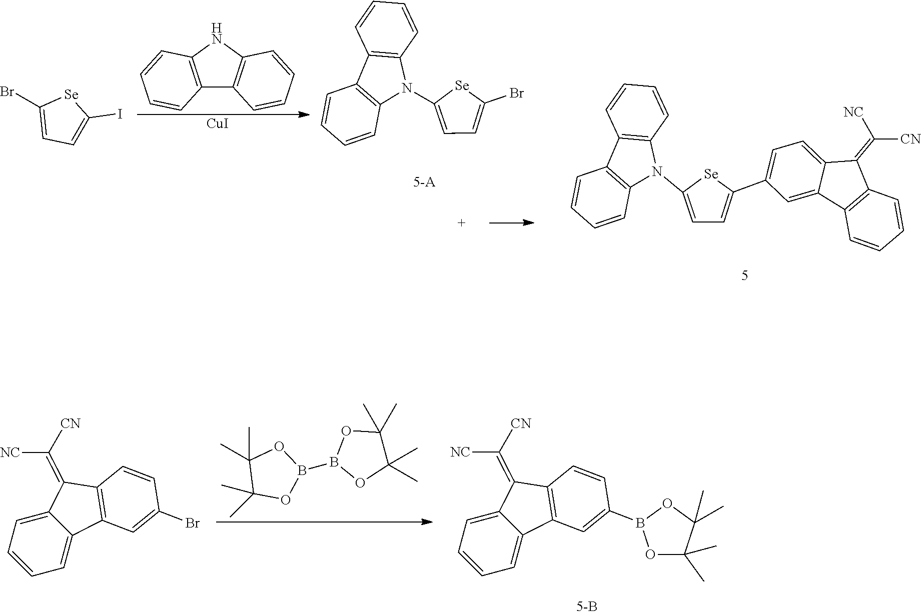

### Synthesis of Intermediate 5-A

Intermediate 5-A was synthesized in substantially the same manner as utilized to synthesize Intermediate 1-A, except that 2-bromo-5-iodoselenophene and carbazole were utilized instead of 2-iodoselenophene and 1-bromo-9H-carbazole during the synthesis of Intermediate 1-A in Synthesis Example 1. The obtained compound was identified by MS/FAB.

C₁₆H₁₀BrNSe: calc. 375.14, found 375.31 .

### Synthesis of Intermediate 5-B

Intermediate 5-B was synthesized in substantially the same manner as utilized to synthesize Intermediate 1-E, except that 2-(3-bromo-9H-fluoren-9-ylidene)propanedinitrile was utilized instead of Intermediate 1-D during the synthesis of Intermediate 1-E in Synthesis Example 1. The obtained compound was identified by MS/FAB.

C₂₂H₁₉BN₂O₂: calc. 354.22, found 354.31 .

### Synthesis of Compound 5

Compound 5 was synthesized in substantially the same manner as utilized to synthesize Compound 1, except that Intermediate 5-A and Intermediate 5-B were respectively utilized instead of 2.19 g (7.14 mmol) of 2-(2-bromo-9H-fluoren-9-ylidene)propanedinitrile and Intermediate 1-E during the synthesis of Compound 1 in Synthesis Example 1. The obtained compound was identified by ¹H NMR (CDCl₃, 400 MHz) and MS/FAB.

¹H NMR: δ=8.56 (d, 1H), 8.29-8.11 (m, 3H), 7.90 (s, 1H), 7.53-7.36 (m, 8H), 7.20-7.10 (m, 4H)

C₃₂H₁₇N₃Se: calc. 522.48, found 523.56 .

### Synthesis Example 4: Synthesis of Compound 7

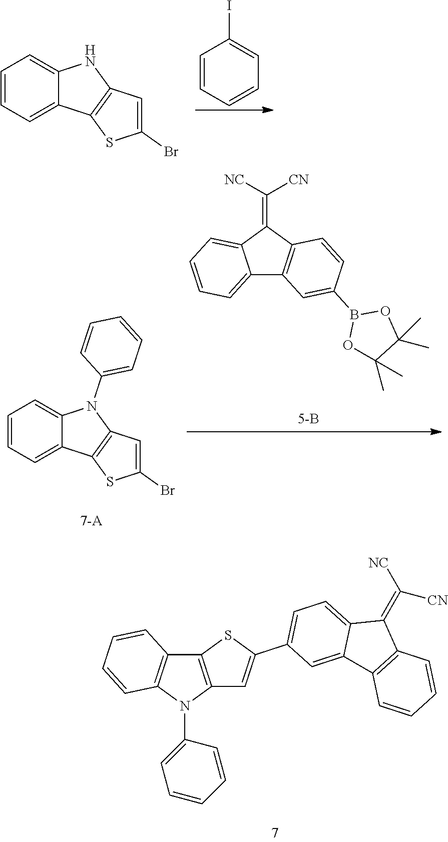 [SMILES: Brc1cc2[nH]c3ccccc3c2s1 ; Ic1ccccc1 ; Brc1cc2c(s1)c1ccccc1n2-c1ccccc1 ; CC1(C)OB(c2ccc3c(c2)-c2ccccc2C3=C(C#N)C#N)OC1(C)C ; N#CC(C#N)=C1c2ccccc2-c2cc(-c3cc4c(s3)c3ccccc3n4-c3ccccc3)ccc21]

### Synthesis of Intermediate 7-A

Intermediate 7-A was synthesized in substantially the same manner as utilized to synthesize Intermediate 1-A, except that iodobenzene and 2-bromo-4H-thieno[3,2-b]indole were respectively utilized instead of 2-iodoselenophene and 1-bromo-9H-carbazole during the synthesis of Intermediate 1-A in Synthesis Example 1. The obtained compound was identified by MS/FAB.

C₁₆H₁₀BrNS: calc. 328.23, found 328.41 .

### Synthesis of Compound 7

Compound 7 was synthesized in substantially the same manner as utilized to synthesize Compound 1, except that Intermediate 7-A and Intermediate 5-B were respectively utilized instead of 2.19 g (7.14 mmol) of 2-(2-bromo-9H-fluoren-9-ylidene)propanedinitrile and Intermediate 1-E during the synthesis of Compound 1 in Synthesis Example 1. The obtained compound was identified by ¹H NMR (CDCl₃, 400 MHz) and MS/FAB.

¹H NMR: δ=8.43-8.40 (m, 1H), 8.29-8.27 (m, 1H), 7.89-7.24 (m, 15H)

C₃₂H₁₇N₃S: calc. 475.57, found 476.02 .

### Synthesis Example 5: Synthesis of Compound 8

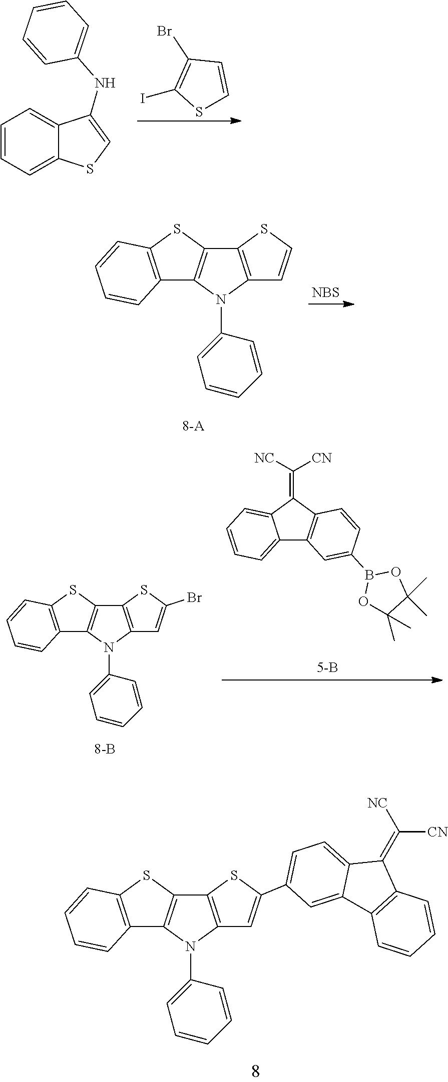

### Synthesis of Intermediate 8-A

4.50 g (20.0 mmol) of N-phenyl-1-benzothiophen-3-amine, 5.78 g (20.0 mmol) of 3-bromo-2-iodothiophene, 0.37 g (0.4 mmol) of Pd₂(dba)₃, 0.08 g (0.4 mmol) of P(t-Bu)₃, and 5.76 g (60.0 mmol) of t-BuOK were dissolved in 90 mL of toluene, and the resultant reaction solution was stirred at 120° C. for 24 hours. The reaction solution was cooled to room temperature, and an organic layer was extracted therefrom three times utilizing 50 mL of water and 50 mL of diethyl ether. The collected organic layer was dried with magnesium sulfate, the residue obtained by evaporating the solvent therefrom was separated and purified by silica gel column chromatography, and 13.36 g (yield: 55%) of Intermediate 8-A was obtained. The obtained compound was identified by MS/FAB.

C₁₈H₁₁NS₂: calc. 305.41, found 305.62 .

### Synthesis of Intermediate 8-B

Intermediate 8-B was synthesized in substantially the same manner as utilized to synthesize Intermediate 1-D, except that Intermediate 8-A was utilized instead of Intermediate 1-C during the synthesis of Intermediate 1-D in Synthesis Example 1. The obtained compound was identified by MS/FAB.

C₁₈H₁₀BrNS₂: calc. 384.31, found 384.33 .

### Synthesis of Compound 8

Compound 8 was synthesized in substantially the same manner as utilized to synthesize Compound 1, except that Intermediate 8-B and Intermediate 5-B were respectively utilized instead of 2-(2-bromo-9H-fluoren-9-ylidene)propanedinitrile and Intermediate 1-E during the synthesis of Compound 1 in Synthesis Example 1. The obtained compound was identified by ¹H NMR (CDCl₃, 400 MHz) and MS/FAB.

¹H NMR: δ=8.27-8.25 (m, 1H), 8.07-8.05 (m, 1H), 7.93-7.10 (m, 15H)

C₃₄H₁₇N₃S2: calc. 531.65, found 531.73 .

### Synthesis Example 6: Synthesis of Compound 10

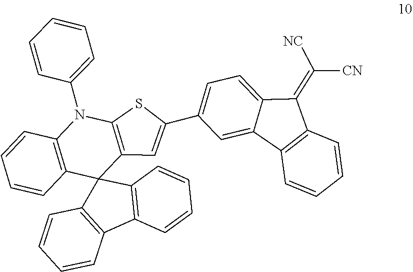 [SMILES: N#CC(C#N)=C1c2ccccc2-c2cc(-c3cc4c(s3)N(c3ccccc3)c3ccccc3C43c4ccccc4-c4ccccc43)ccc21]

Compound 10 was synthesized in substantially the same manner as utilized to synthesize Compound 1, except that, in Synthesis Example 1, 2-iodothiophene was utilized instead of 2-iodoselenophene during the synthesis of Intermediate 1-A, 9H-fluoren-9-one was utilized instead of acetone during the synthesis of Intermediate 1-B, and 2-(3-bromo-9H-fluoren-9-ylidene)propanedinitrile was utilized instead of 2-(2-bromo-9H-fluoren-9-ylidene)propanedinitrile during the synthesis of Compound 1. The obtained compound was identified by ¹H NMR (CDCl₃, 400 MHz) and MS/FAB.

¹H NMR: δ=8.29-8.25 (m, 1H), 7.90-7.82 (m, 5H), 7.72-7.70 (m, 1H), 7.53-6.95 (m, 18H)

C₄₅H₂₅N₃S: calc. 522.48, found 523.56 .

### Synthesis Example 7: Synthesis of Compound 13

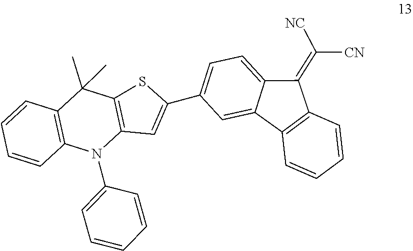 [SMILES: CC1(C)c2ccccc2N(c2ccccc2)c2cc(-c3ccc4c(c3)-c3ccccc3C4=C(C#N)C#N)sc21]

Compound 13 was synthesized in substantially the same manner as utilized to synthesize Compound 1, except that 2-iodothiophene and 2-bromo-N-phenylaniline were respectively utilized instead of 2-iodoselenophene and 1-bromo-9H-carbazole during the synthesis of Intermediate 1-A in Synthesis Example 1. The obtained compound was identified by ¹H NMR (CDCl₃, 400 MHz) and MS/FAB.

¹H NMR: δ=8.29-8.25 (m, 2H), 7.89-7.72 (m, 3H), 7.53-7.37 (m, 3H), 7.24-6.95 (m, 9H), 1.72 (s, 6H)

C₃₅H₂₃N₃S: calc. 517.65, found 518.72 .

### Synthesis Example 8: Synthesis of Compound 16

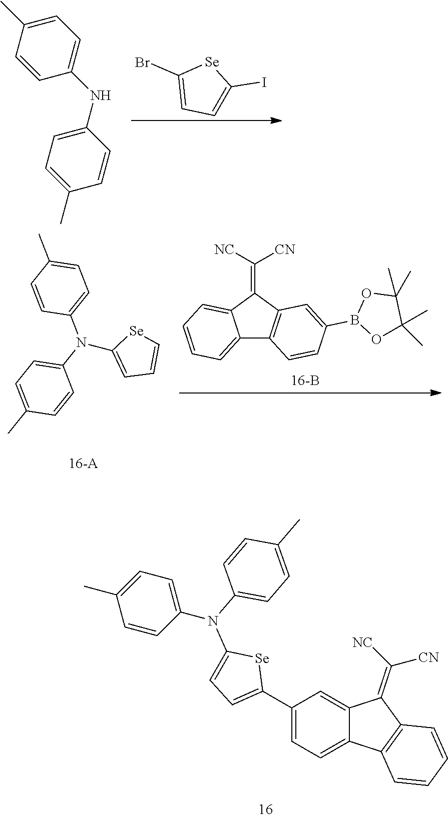

### Synthesis of Intermediate 16-A

Intermediate 16-A was synthesized in substantially the same manner as utilized to synthesize Intermediate 8-A, except that 4,4′-dimethyldiphenylamine and 2-bromo-5-iodothiophene were respectively utilized instead of N-phenyl-1-benzothiophen-3-amine and 3-bromo-2-iodothiophene during the synthesis of Intermediate 8-A in Synthesis Example 5. The obtained compound was identified by MS/FAB.

C₁₈H₁₇NSe: calc. 326.31, found 326.43 .

### Synthesis of Compound 16

Compound 16 was synthesized in substantially the same manner as utilized to synthesize Compound 1, except that Intermediate 16-A and Intermediate 16-B were respectively utilized instead of 2-(2-bromo-9H-fluoren-9-ylidene)propanedinitrile and Intermediate 1-E during the synthesis of Compound 1 in Synthesis Example 1. The obtained compound was identified by ¹H NMR (CDCl₃, 400 MHz) and MS/FAB.

¹H NMR: δ=8.29-8.27 (m, 1H), 7.67-7.65 (m, 1H), 7.53-7.37 (m, 7H), 7.15-7.12 (m, 6H), 6.86 (s, 1H), 6.59 (s, 1H), 2.32 (s, 6H)

C₃₄H₂₃N₃Se: calc. 552.55, found 552.73 .

### Synthesis Example 9: Synthesis of Compound 29

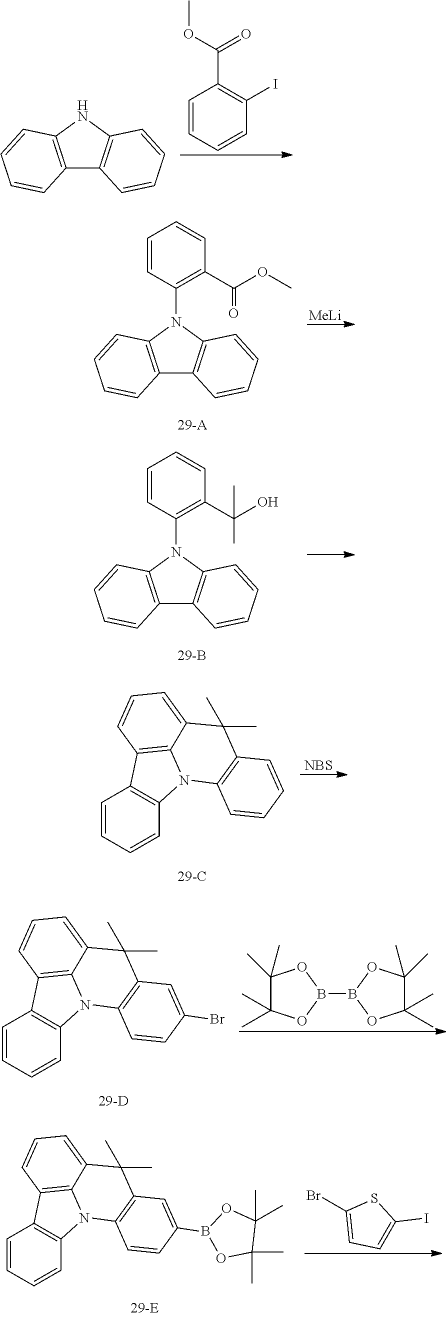

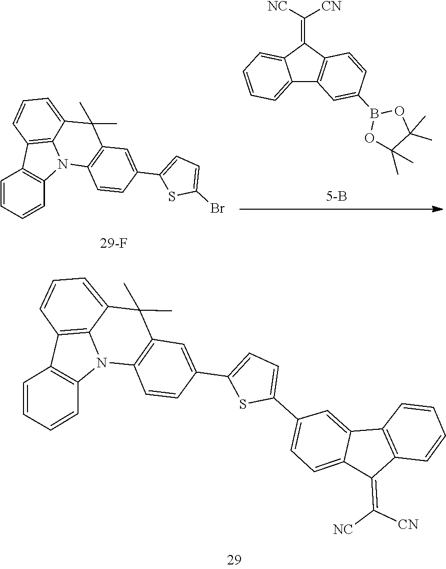 [SMILES: CC1(C)c2cc(-c3ccc(Br)s3)ccc2-n2c3ccccc3c3cccc1c32 ; CC1(C)OB(c2ccc3c(c2)-c2ccccc2C3=C(C#N)C#N)OC1(C)C ; CC1(C)c2cc(-c3ccc(-c4ccc5c(c4)-c4ccccc4C5=C(C#N)C#N)s3)ccc2-n2c3ccccc3c3cccc1c32]

### Synthesis of Intermediate 29-A

Intermediate 29-A was synthesized in substantially the same manner as utilized to synthesize Intermediate 1-A, except that methyl 2-iodobenzoate and carbazole were respectively utilized instead of 2-iodoselenophene and 1-bromo-9H-carbazole during the synthesis of Intermediate 1-A in Synthesis Example 1. The obtained compound was identified by MS/FAB.

C₂₀H₁₅NO₂: calc. 301.35, found 301.45 .

### Synthesis of Intermediate 29-B

2.15 g (7.14 mmol) of Intermediate 29-A was dissolved in 100 mL of ether and cooled to −78° C., methyllithium (1.6 M in ether, 78 mL, 124.4 mmol) was added thereto, and the resultant reaction solution was stirred for 1 hours. The reaction solution was slowly heated to room temperature and further stirred for 4 hours. When the reaction was completed, an organic layer was extracted therefrom utilizing 200 mL of water, and the organic layer was dried under reduced pressure. The resultant product was recrystallized utilizing ethanol/acetone (volume ratio of 1:1) to obtain 1.441 g (yield: 67%) of Intermediate 29-B. The obtained compound was identified by MS/FAB.

C₂₁H₁₉NO: calc. 301.39, found 301.62 .

### Synthesis of Intermediate 29-C

Intermediate 29-C was synthesized in substantially the same manner as utilized to synthesize Intermediate 1-C, except that Intermediate 29-B was utilized instead of Intermediate 1-B during the synthesis of Intermediate 1-C in Synthesis Example 1. The obtained compound was identified by MS/FAB.

C₂₁H₁₇N: calc. 283.37, found 283.41 .

### Synthesis of Intermediate 29-D

Intermediate 29-D was synthesized in substantially the same manner as utilized to synthesize Intermediate 1-D, except that Intermediate 29-C was utilized instead of Intermediate 1-C during the synthesis of Intermediate 1-D in Synthesis Example 1. The obtained compound was identified by MS/FAB.

C₂₁H₁₆BrN: calc. 362.27, found 362.34 .

### Synthesis of Intermediate 29-E

Intermediate 29-E was synthesized in substantially the same manner as utilized to synthesize Intermediate 1-E, except that Intermediate 29-D was utilized instead of Intermediate 1-D during the synthesis of Intermediate 1-E in Synthesis Example 1. The obtained compound was identified by MS/FAB.

C₂₇H₂₈BNO₂: calc. 409.34, found 409.39.

### Synthesis of Intermediate 29-F

Intermediate 29-F was synthesized in substantially the same manner as utilized to synthesize Compound 1, except that 2-bromo-5-iodothiophene and Intermediate 29-E were respectively utilized instead of 2-(2-bromo-9H-fluoren-9-ylidene)propanedinitrile and Intermediate 1-E during the synthesis of Compound 1 in Synthesis Example 1. The obtained compound was identified by MS/FAB.

C₂₅H₁₈BrNS: calc. 444.39, found 444.62 .

### Synthesis of Compound 29

Compound 29 was synthesized in substantially the same manner as utilized to synthesize Compound 1, except that Intermediate 29-F and Intermediate 5-B were respectively utilized instead of 2-(2-bromo-9H-fluoren-9-ylidene)propanedinitrile and Intermediate 1-E during the synthesis of Compound 1 in Synthesis Example 1. The obtained compound was identified by ¹H NMR (CDCl₃, 400 MHz) and MS/FAB.

¹H NMR: δ=8.57-8.55 (m, 1H), 8.29-8.27 (m, 1H), 8.09-8.07 (m, 1H), 7.94-7.72 (m, 7H), 7.53-7.35 (m, 7H), 7.06-7.07 (m, 2H), 1.69 (s, 6H)

C₄₁H₂₅N₃S: calc. 591.73, found 591.79 .

### Synthesis Example 10: Synthesis of Compound 39

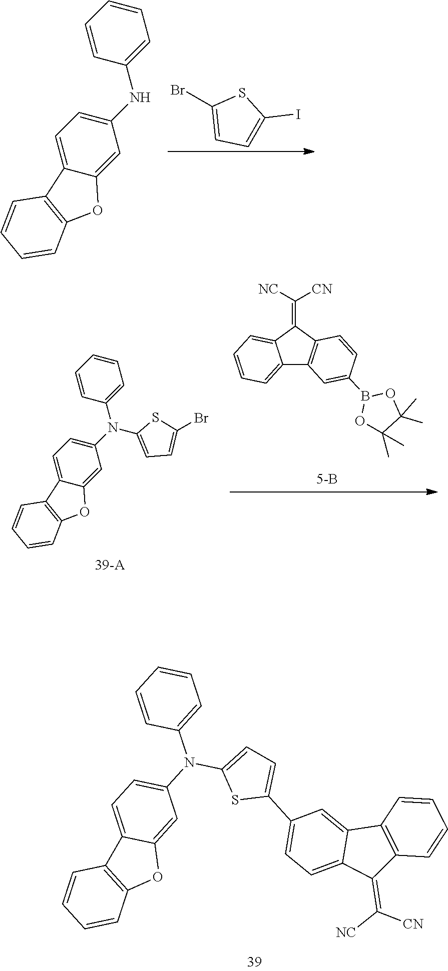 [SMILES: c1ccc(Nc2ccc3c(c2)oc2ccccc23)cc1 ; Brc1ccc(I)s1 ; Brc1ccc(N(c2ccccc2)c2ccc3c(c2)oc2ccccc23)s1 ; CC1(C)OB(c2ccc3c(c2)-c2ccccc2C3=C(C#N)C#N)OC1(C)C ; N#CC(C#N)=C1c2ccccc2-c2cc(-c3ccc(N(c4ccccc4)c4ccc5c(c4)oc4ccccc45)s3)ccc21]

### Synthesis of Intermediate 39-A

Intermediate 39-A was synthesized in substantially the same manner as utilized to synthesize Intermediate 8-A, except that N-phenyldibenzo[b,d]furan-3-amine and 2-bromo-5-iodothiophene were respectively utilized instead of N-phenyl-1-benzothiophen-3-amine and 3-bromo-2-iodothiophene during the synthesis of Intermediate 8-A in Synthesis Example 5. The obtained compound was identified by MS/FAB.

C₂₂H₁₄NOSBr: calc. 420.34, found 420.43 .

### Synthesis of Compound 39

Compound 39 was synthesized in substantially the same manner as utilized to synthesize Compound 1, except that Intermediate 39-A and Intermediate 5-B were respectively utilized instead of 2-(2-bromo-9H-fluoren-9-ylidene)propanedinitrile and Intermediate 1-E during the synthesis of Compound 1 in Synthesis Example 1. The obtained compound was identified by ¹H NMR (CDCl₃, 400 MHz) and MS/FAB.

¹H NMR: δ=8.29-8.27 (m, 1H), 8.03-7.72 (m, 6H), 7.53-7.24 (m, 8H), 7.08-6.85 (m, 5H), 6.15-6.13 (m, 1H)

C₃₈H₂₁N₃OS: calc. 567.67, found 567.41 .

### Synthesis Example 11: Synthesis of Compound 47

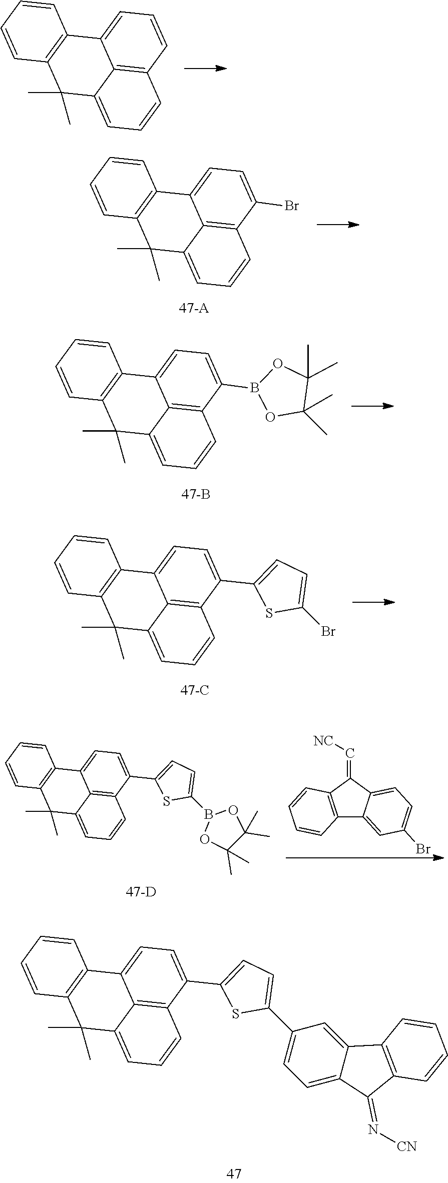

### Synthesis of Intermediate 47-A

Intermediate 47-A was synthesized in substantially the same manner as utilized to synthesize Intermediate 1-D, except that 7H-benz[de]anthracene,7,7-dimethyl was utilized instead of Intermediate 1-C during the synthesis of Intermediate 1-D in Synthesis Example 1. The obtained compound was identified by MS/FAB.

C₁₉H₁₅Br: calc. 323.23, found 323.44 .

### Synthesis of Intermediate 47-B

Intermediate 47-B was synthesized in substantially the same manner as utilized to synthesize Intermediate 1-E, except that Intermediate 47-A was utilized instead of Intermediate 1-D during the synthesis of Intermediate 1-E in Synthesis Example 1. The obtained compound was identified by MS/FAB.

C₂₅H₂₇BO₂: calc 370.30, found 370.40.

### Synthesis of Intermediate 47-C

Intermediate 47-C was synthesized in substantially the same manner as utilized to synthesize Compound 1, except that 2-bromo-5-iodothiophene and Intermediate 47-B were respectively utilized instead of 2-(2-bromo-9H-fluoren-9-ylidene)propanedinitrile and Intermediate 1-E during the synthesis of Compound 1 in Synthesis Example 1. The obtained compound was identified by MS/FAB.

C₂₃H₁₇BrS: calc. 405.35, found 405.62 .

### Synthesis of Intermediate 47-D

Intermediate 47-D was synthesized in substantially the same manner as utilized to synthesize Intermediate 1-E, except that Intermediate 47-C was utilized instead of Intermediate 1-D during the synthesis of Intermediate 1-E in Synthesis Example 1. The obtained compound was identified by MS/FAB.

C₂₉H₂₉BO₂S: calc 452.42, found 452.65 .

### Synthesis of Compound 47

Compound 47 was synthesized in substantially the same manner as utilized to synthesize Compound 1, except that (E)-N-(3-bromo-9H-fluoren-9-ylidene)cyanamide and Intermediate 47-D were respectively utilized instead of 2-(2-bromo-9H-fluoren-9-ylidene)propanedinitrile and Intermediate 1-E during the synthesis of Compound 1 in Synthesis Example 1. The obtained compound was identified by 1H NMR (CDCl₃, 400 MHz) and MS/FAB.

¹H NMR: δ=8.87-8.85 (m, 1H), 8.32-8.30 (m, 1H), 8.18 (s, 1H), 8.11-8.00 (m, 3H), 7.85-7.60 (m, 6H), 7.44-7.37 (m, 3H), 7.30-7.28 (m, 2H), 6.91-6.89 (m, 1H), 1.82 (s, 6H)

C₃₇H₂₄N₂S: calc. 528.67, found 528.81.

### Synthesis Example 12: Synthesis of Compound 48

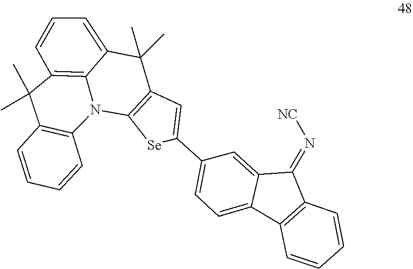 [SMILES: CC1(C)c2ccccc2N2c3[se]c(-c4ccc5c(c4)/C(=N\C#N)c4ccccc4-5)cc3C(C)(C)c3cccc1c32]

Compound 48 was synthesized in substantially the same manner as utilized to synthesize Compound 1, except that 4-bromo-9,9-dimethyl-9H-acridine and (E)-N-(3-bromo-9H-fluoren-9-ylidene)cyanamide were respectively utilized instead of 1-bromo-9H-carbazole and 2-(2-bromo-9H-fluoren-9-ylidene)propanedinitrile during the synthesis of Intermediate 1-A in Synthesis Example 1. The obtained compound was identified by ¹H NMR (CDCl₃, 400 MHz) and MS/FAB.

¹H NMR: δ=8.00-7.96 (m, 2H), 7.85-7.60 (m, 4H), 7.48-7.46 (m, 2H), 7.32-7.30 (m, 1H), 7.19-7.14 (m, 5H), 6.95-6.93 (m, 1H), 1.69 (s, 6H), 1.46 (s, 6H)

C₃₆H₂₇N₃Se: calc. 580.60, found 580.75 .

### Synthesis Example 13: Synthesis of Compound R05

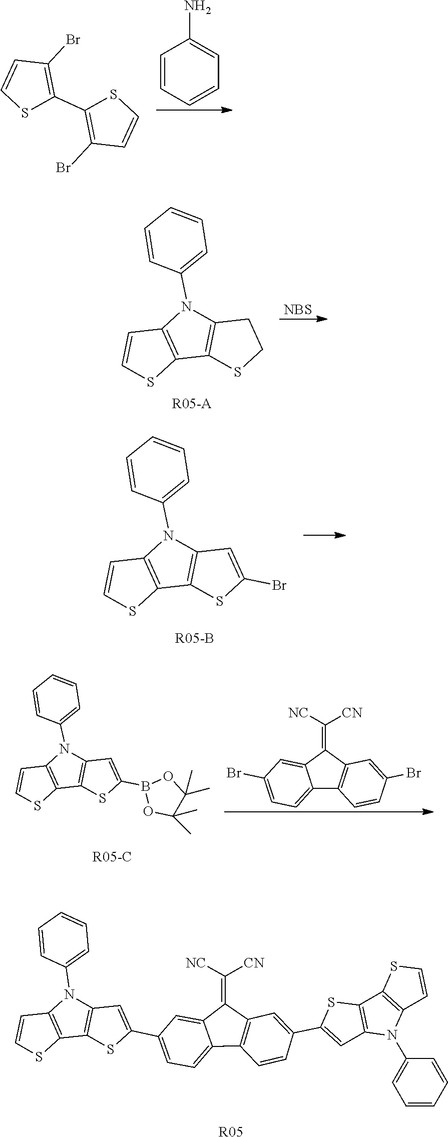

### Synthesis of Intermediate R05-A

In a nitrogen atmosphere, 0.65 g (0.71 mmol) of Pd₂(dba)₃ and 0.21 g (0.71 mmol) of a P(^{t}-Bu)₃HBF₄ ligand were added to 90 mL of toluene. 2.05 g (21.3 mmol) of NaO^{t}Bu and 2.30 g (7.1 mmol) of 3,3′-dibromo-2,2′-bithiophene were successively added thereto. After 5 minutes, arylamine (9.94 mmol) was added thereto, and the resultant reaction solution was stirred at 125° C. for 6 hours. The reaction solution was cooled to room temperature, and an organic layer was extracted therefrom three times utilizing 50 mL of water and 50 mL of diethyl ether. The collected organic layer was dried with magnesium sulfate, the residue obtained by evaporating the solvent therefrom was separated and purified by silica gel column chromatography, and 1.0 g (yield: 55%) of Intermediate R05-A was obtained. The obtained compound was identified by MS/FAB.

C₁₄H9NS₂: calc. 255.35, found 255.41 .

### Synthesis of Intermediate R05-B

Intermediate R05-B was synthesized in substantially the same manner as utilized to synthesize Intermediate 1-D, except that Intermediate R05-A was utilized instead of Intermediate 1-C during the synthesis of Intermediate 1-D in Synthesis Example 1. The obtained compound was identified by MS/FAB.

C₁₄H₈BrNS₂: calc. 334.25, found 334.29 .

### Synthesis of Intermediate R05-C

Intermediate R05-C was synthesized in substantially the same manner as utilized to synthesize Intermediate 1-E, except that Intermediate R05-B was utilized instead of Intermediate 1-D during the synthesis of Intermediate 1-E in Synthesis Example 1. The obtained compound was identified by MS/FAB.

C₂₀H₂₀BNO₂S₂: calc 381.32, found 381.40.

### Synthesis of Compound R05

Compound R05 was synthesized in substantially the same manner as utilized to synthesize Compound 1, except that 2-(2,7-dibromo-9H-fluoren-9-ylidene)malonitrile and Intermediate R05-C were respectively utilized instead of 2-(2-bromo-9H-fluoren-9-ylidene)propanedinitrile and Intermediate 1-E during the synthesis of Compound 1 in Synthesis Example 1. The obtained compound was identified by 1H NMR (CDCl₃, 400 MHz) and MS/FAB.

¹H NMR: δ=8.16-8.14 (m, 2H), 7.91-7.84 (m, 4H), 7.62-7.50 (m, 10H), 7.31-7.29 (m, 2H), 7.12-7.08 (m, 4H)

C₄₄H₂₂N4S4: calc. 734.93, found 734.96 .

### Synthesis Example 14: Synthesis of Compound R06

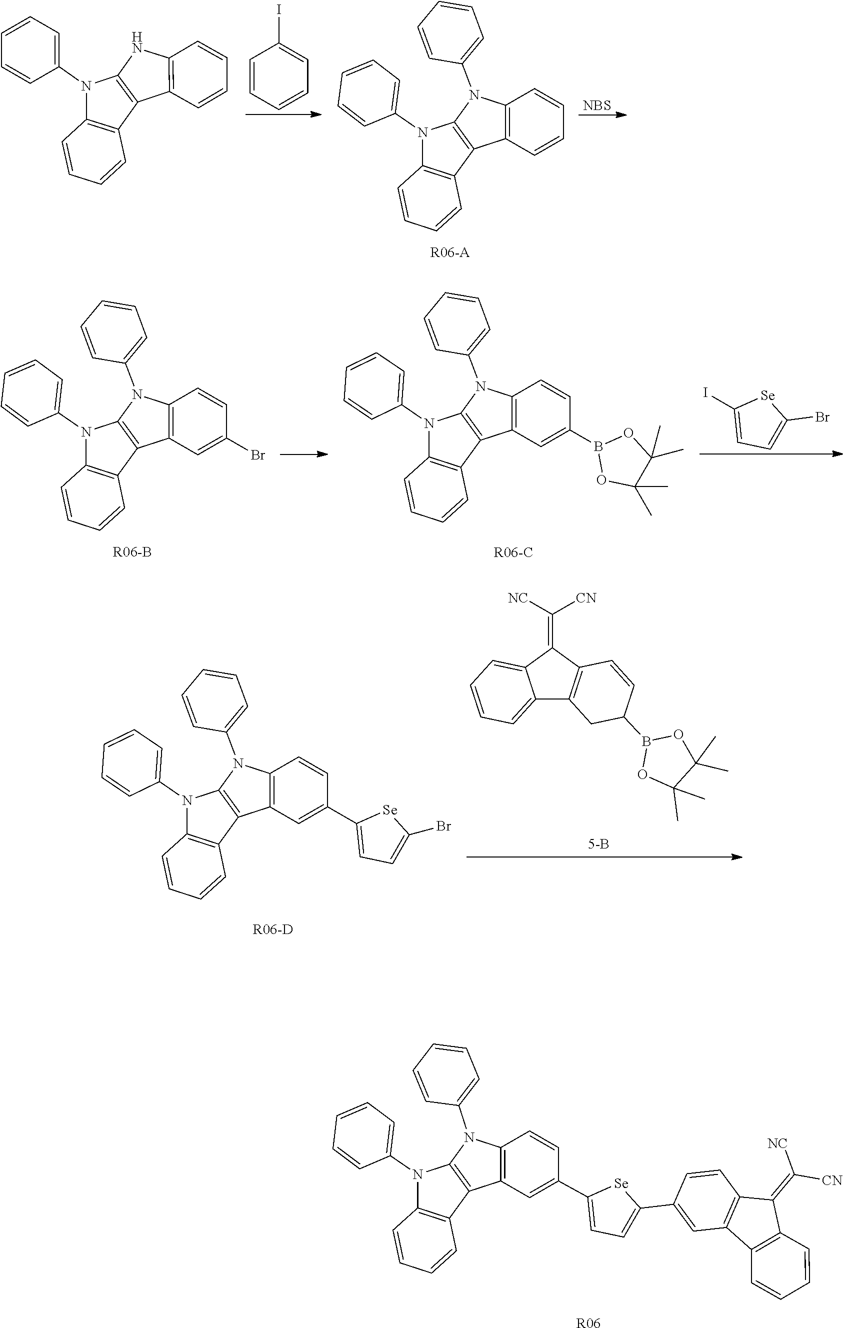

### Synthesis of Intermediate R06-A

Intermediate R06-A was synthesized in substantially the same manner as utilized to synthesize Intermediate 8-A, except that 5,6-dihydro-5-phenylindolo[2,3-b]indole and iodobenzene were respectively utilized instead of N-phenyl-1-benzothiophen-3-amine and 3-bromo-2-iodothiophene during the synthesis of Intermediate 8-A in Synthesis Example 5. The obtained compound was identified by MS/FAB.

C₂₆H₁₈N₂: calc. 358.44, found 358.47 .

### Synthesis of Intermediate R06-B

Intermediate R06-B was synthesized in substantially the same manner as utilized to synthesize Intermediate 1-D, except that Intermediate R06-A was utilized instead of Intermediate 1-C during the synthesis of Intermediate 1-D in Synthesis Example 1. The obtained compound was identified by MS/FAB.

C₂₆H₁₇BrN₂: calc. 437.34, found 437.35 .

### Synthesis of Intermediate R06-C

Intermediate R06-C was synthesized in substantially the same manner as utilized to synthesize Intermediate 1-E, except that Intermediate R06-B was utilized instead of Intermediate 1-D during the synthesis of Intermediate 1-E in Synthesis Example 1. The obtained compound was identified by MS/FAB.

C₃₂H₂₉BN₂O₂: calc 484.41, found 484.44 .

### Synthesis of Intermediate R06-D

Intermediate R06-D was synthesized in substantially the same manner as utilized to synthesize Compound 1, except that 2-bromo-5-iodoselenophene and Intermediate R06-C were respectively utilized instead of 2-(2-bromo-9H-fluoren-9-ylidene)propanedinitrile and Intermediate 1-E during the synthesis of Compound 1 in Synthesis Example 1. The obtained compound was identified by MS/FAB.

C₃₀H₁₉BrN₂Se: calc. 566.37, found 566.39 .

### Synthesis of Compound R06

Compound R06 was synthesized in substantially the same manner as utilized to synthesize Compound 1, except that Intermediate R06-D and Intermediate 5-B were respectively utilized instead of 2-(2-bromo-9H-fluoren-9-ylidene)propanedinitrile and Intermediate 1-E during the synthesis of Compound 1 in Synthesis Example 1. The obtained compound was identified by ¹H NMR (CDCl₃, 400 MHz) and MS/FAB.

¹H NMR: δ=8.55-8.53 (m, 1H), 8.47 (s, 1H), 8.29 (d, 1H), 7.94-7.89 (m, 3H), 7.62-7.37 (m, 18H), 7.18-7.16 (m, 1H), 6.96-6.94 (m, 1H)

C₄₆H₂₆N4Se: calc. 713.71 found 713.73 .

### Synthesis Example 15: Synthesis of Compound R07

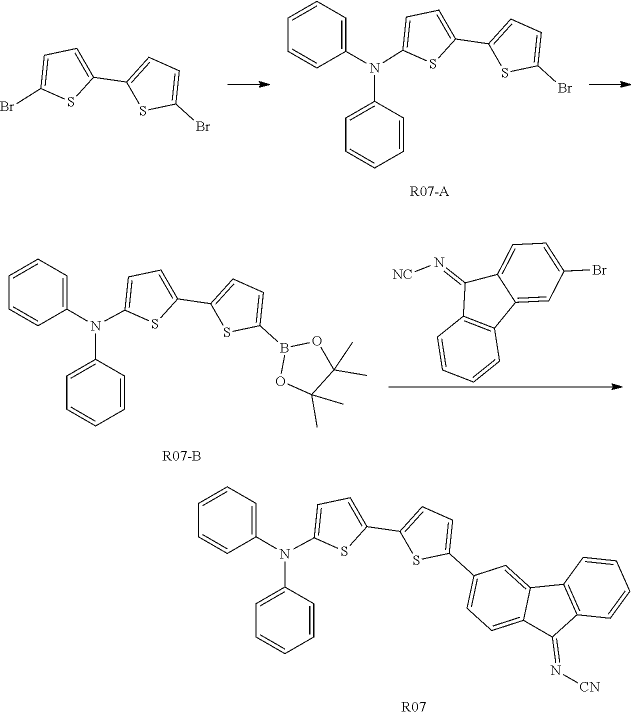

### Synthesis of Intermediate R07-A

Intermediate R07-A was synthesized in substantially the same manner as utilized to synthesize Intermediate 8-A, except that N,N-diphenylamine and 5,5′-dibromo-2,2′-bithiophene were respectively utilized instead of N-phenyl-1-benzothiophen-3-amine and 3-bromo-2-iodothiophene during the synthesis of Intermediate 8-A in Synthesis Example 5. The obtained compound was identified by MS/FAB.

C₂₀H₁₄BrNS₂: calc. 412.36, found 412.39 .

### Synthesis of Intermediate R07-B

Intermediate R07-B was synthesized in substantially the same manner as utilized to synthesize Intermediate 1-E, except that Intermediate R07-A was utilized instead of Intermediate 1-D during the synthesis of Intermediate 1-E in Synthesis Example 1. The obtained compound was identified by MS/FAB.

C₂₆H₂₆BNO₂S₂: calc 459.43, found 459.46 .

### Synthesis of Compound R07

Compound R07 was synthesized in substantially the same manner as utilized to synthesize Compound 1, except that (E)-N-(3-bromo-9H-fluoren-9-ylidene)cyanamide and Intermediate R07-B were respectively utilized instead of 1-bromo-9H-carbazole and 2-(2-bromo-9H-fluoren-9-ylidene)propanedinitrile during the synthesis of Intermediate 1-A in Synthesis Example 1. The obtained compound was identified by ¹H NMR (CDCl₃, 400 MHz) and MS/FAB.

¹H NMR: δ=8.32-8.30 (m, 1H), 8.18 (s, 1H), 8.11 (d, 1H), 8.00-7.98 (m, 1H), 7.85-7.83 (m, 1H), 7.69-7.60 (m, 2H), 7.51-7.49 (m, 1H), 7.34-7.32 (m, 1H), 7.24-7.20 (m, 4H), 7.08-7.00 (m, 7H), 6.05-6.02 (m, 1H)

C₃₄H₂₁N₃S2: calc. 535.68, found 535.75.

¹H NMR and MS/FAB of the compounds synthesized according to Synthesis Examples are shown in Table 1.

TABLE 1

| | | MS/FAB | |
|---|---|---|---|
| No. | ¹H NMR (CDCl₃, 400 MHz) | found | calc. |
| Compound | δ = 8.57-8.55 (m, 1H), 8.29-8.11 (m, 3H), | 562.75 | 562.55 |
| 1 | 7.71-7.67 (m, 2H), 7.53-7.41 (m, 4H), 7.36- | | |
| | 7.08 (m, 4H), 6.86 (s, 1H), 1.46 (s, 6H) | | |
| Compound | δ = 8.29-8.25 (m, 1H), 7.53-7.41 (m, 5H), | 604.75 | 604.63 |
| 3 | 7.37-7.14 (m, 7H), 6.95-6.86 (m, 2H), 1.69 | | |
| | (s, 6H), 1.46 (s, 6H) | | |
| Compound | δ = 8.56 (d, 1H), 8.29-8.11 (m, 3H), 7.90 (s, | 523.56 | 522.48 |
| 5 | 1H), 7.53-7.36 (m, 8H), 7.20-7.10 (m, 4H) | | |
| Compound | δ = 8.43-8.40 (m, 1H), 8.29-8.27 (m, 1H), | 476.02 | 475.57 |
| 7 | 7.89-7.24 (m, 15H) | | |
| Compound | δ = 8.27-8.25 (m, 1H), 8.07-8.05 (m, 1H), | 531.73 | 531.65 |
| 8 | 7.93-7.10 (m, 15H) | | |
| Compound | δ = 8.29-8.25 (m, 1H), 7.90-7.82 (m, 5H), | 523.56 | 522.48 |
| 10 | 7.72-7.70 (m, 1H), 7.53-6.95 (m, 18H) | | |
| Compound | δ = 8.29-8.25 (m, 2H), 7.89-7.72 (m, 3H), | 518.72 | 517.65 |
| 13 | 7.53-7.37 (m, 3H), 7.24-6.95 (m, 9H), | | |
| | 1.72 (s, 6H) | | |
| Compound | δ = 8.29-8.27 (m, 1H), 7.67-7.65 (m, 1H), | 552.73 | 552.55 |
| 16 | 7.53-7.37 (m, 7H), 7.15-7.12 (m, 6H), 6.86 | | |
| | (s, 1H), 6.59 (s, 1H), 2.32 (s, 6H) | | |
| Compound | δ = 8.57-8.55 (m, 1H), 8.29-8.27 (m, 1H), | 597.79 | 597.73 |
| 29 | 8.09-8.07 (m, 1H), 7.94-7.72 (m, 7H), 7.53- | | |
| | 7.35 (m, 7H), 7.06-7.07 (m, 2H), 1.69 (s, 6H) | | |
| Compound | δ = 8.29-8.27 (m, 1H), 8.03-7.72 (m, 6H), | 567.41 | 567.67 |
| 39 | 7.53-7.24 (m, 8H), 7.08-6.85 (m, 5H), 6.15- | | |
| | 6.13 (m, 1H) | | |
| Compound | δ = 8.87-8.85 (m, 1H), 8.32-8.30 (m, 1H), | 528.81 | 528.67 |
| 47 | 8.18 (s, 1H), 8.11-8.00 (m, 3H), 7.85-7.60 | | |
| | (m, 6H), 7.44-7.37 (m, 3H), 7.30-7.28(m, | | |
| | 2H), 6.91-6.89 (m, 1H), 1.82(s, 6H) | | |
| Compound | δ = 8.00-7.96 (m, 2H), 7.85-7.60 (m, 4H), | 580.75 | 580.60 |
| 48 | 7.48-7.46 (m, 2H), 7.32-7.30 (m, 1H), 7.19- | | |
| | 7.14 (m, 5H), 6.95-6.93 (m, 1H), 1.69 (s, | | |
| | 6H), 1.46 (s, 6H) | | |
| Compound | δ = 8.16-8.14 (m, 2H), 7.91-7.84 (m, 4H), | 734.96 | 734.93 |
| R05 | 7.62-7.50 (m, 10H), 7.31-7.29 (m, 2H), 7.12- | | |
| | 7.08 (m, 4H) | | |
| Compound | δ = 8.55-8.53 (m, 1H), 8.47 (s, 1H), 8.29 (d, | 713.73 | 713.71 |
| R06 | 1H), 7.94-7.89 (m, 3H), 7.62-7.37 (m, 18H), | | |
| | 7.18-7.16 (m, 1H), 6.96-6.94 (m, 1H) | | |
| Compound | δ = 8.32-8.30 (m, 1H), 8.18 (s, 1H), 8.11 (d, | 535.75 | 535.68 |
| R07 | 1H), 8.00-7.98 (m, 1H), 7.85-7.83(m, 1H), | | |
| | 7.69-7.60 (m, 2H), 7.51-7.49 (m, 1H), 7.34- | | |
| | 7.32 (m, 1H), 7.24-7.20 (m, 4H), 7.08-7.00 | | |
| | (m, 7H), 6.05-6.02 (m, 1H) | | |

### Evaluation Example 1

The LUMO and HOMO values of the compounds of Synthesis Examples and Comparative Compounds were measured utilizing the method described in Table 2. The results are shown in Table 3.

TABLE 2

| | |
|---|---|
| HOMO | By utilizing cyclic voltammetry (CV) (electrolyte: 0.1M |
| energy | Bu₄NPF₆/solvent: dimethylformamide (DMF)/electrode: |
| level | 3-electrode system (working electrode: GC, reference |
| evaluation | electrode: Ag/AgCl, and auxiliary electrode: Pt)), the |
| method | potential (V)-current (A) graph of each compound was |
| | obtained, and then, from the oxidation onset of the |
| | graph, the HOMO energy level of each compound was |
| | calculated. |
| LUMO | By utilizing cyclic voltammetry (CV) (electrolyte: 0.1M |
| energy | Bu₄NPF₆/solvent: dimethylformamide (DMF)/electrode: |
| level | 3-electrode system (working electrode: GC, reference |
| evaluation | electrode: Ag/AgCl, and auxiliary electrode: Pt)), the |
| method | potential (V)-current (A) graph of each compound was |
| | obtained, and then, from the reduction onset of the |
| | graph, the LUMO energy level of each compound was |
| | calculated. |

TABLE 3

| Compound No. | HOMO (eV) | LUMO (eV) |
|---|---|---|
| Compound 1 | −5.72 | −2.95 |
| Compound 3 | −5.71 | −2.94 |
| Compound 5 | −5.65 | −2.85 |
| Compound 7 | −5.64 | −2.85 |
| Compound 8 | −5.62 | −2.83 |
| Compound 10 | −5.70 | −2.95 |
| Compound 13 | −5.69 | −2.96 |
| Compound 16 | −5.58 | −2.79 |
| Compound 29 | −5.68 | −2.89 |
| Compound 39 | −5.55 | −2.77 |
| Compound 47 | −5.67 | −2.88 |
| Compound 48 | −5.70 | −2.90 |
| Compound | −5.65 | −2.85 |
| R05 | | |
| Compound | −5.64 | −2.84 |
| R06 | | |
| Compound | −5.51 | −3.70 |
| R07 | | |
| Comparative | −5.55 | −2.83 |
| Compound 1 | | |
| Comparative | −5.55 | −3.42 |
| Compound 2 | | |

The structures of Comparative Compounds 1 and 2 are as follows.

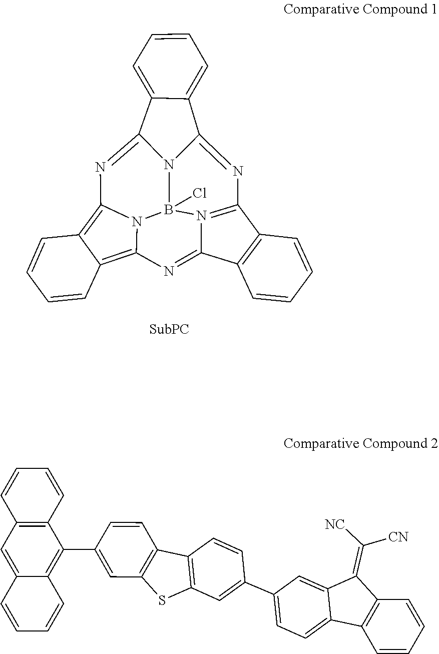

### Example 1

A substrate having an ITO anode deposited thereon was cut to a size of 50 mm×50 mm×0.7 mm. The substrate was ultrasonically cleaned in isopropyl alcohol and pure water each for 5 minutes. Then, ultraviolet rays were irradiated onto the substrate for 30 minutes, and the substrate was cleaned by exposure to ozone. The cleaned substrate was loaded onto a vacuum deposition apparatus.

Compound 2-TNATA was vacuum-deposited on the ITO substrate to form a hole injection layer having a thickness of 600 Å. Then, 4,4′-bis[N-(1-naphthyl)-N-phenylamino]biphenyl (hereinafter, referred to as NPB) was vacuum-deposited on the hole injection layer to form a hole transport layer having a thickness of 300 Å. An auxiliary layer having a thickness of 300 Å was formed on the hole transport layer.

Compound 1 and Compound N₁, which is a photoelectric conversion material, were co-deposited at a weight ratio of 20:80 on the auxiliary layer to form a light-receiving layer having a thickness of 500 Å. Alq₃ was deposited on the light-receiving layer to form an electron transport layer having a thickness of 300 Å. LiF, which is an alkali metal halide, was deposited on the electron transport layer to form an electron injection layer having a thickness of 10 Å. Then, Al was vacuum-deposited thereon to form a LiF/Al cathode having a thickness of 3,000 Å, thereby completing the manufacture of a light-receiving device.

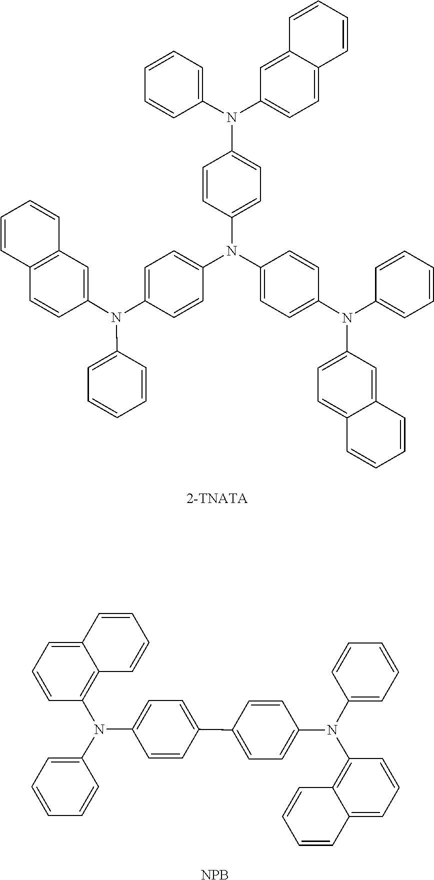 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

### Examples 2 to 15 and Comparative Examples 1 and 2

Light-receiving devices were manufactured in substantially the same manner as in Example 1, except that the compounds shown in Table 4 were each utilized instead of Compound 1 in forming the light-receiving layer.

### Evaluation Example 2

The light-receiving devices manufactured according to Examples 1 to 15 and Comparative Examples 1 and 2 were irradiated with light, and the external quantum efficiency (EQE, %) according to wavelength was measured for each device. The external quantum efficiency refers to the ratio of electrical energy generated from light. The results are shown in Table 4.

TABLE 4

| | | Light- | | |
|---|---|---|---|---|
| | | absorbing | | |
| | | material of | External | Light- |
| | | light- | quantum | absorption |
| | | receiving | efficiency | wavelength |
| | | layer | (EQE, %) | (λₘₐₓ, nm) |
| Example | 1 | Compound 1 | 58 | 530 |
| | 2 | Compound 3 | 52 | 530 |
| | 3 | Compound 5 | 48 | 528 |
| | 4 | Compound 7 | 53 | 530 |
| | 5 | Compound 8 | 55 | 520 |
| | 6 | Compound | 44 | 536 |
| | | 10 | | |
| | 7 | Compound | 34 | 538 |
| | | 13 | | |
| | 8 | Compound | 34 | 539 |
| | | 16 | | |
| | 9 | Compound | 48 | 531 |
| | | 29 | | |
| | 10 | Compound | 49 | 531 |
| | | 39 | | |
| | 11 | Compound | 51 | 530 |
| | | 47 | | |
| | 12 | Compound | 56 | 531 |
| | | 48 | | |
| | 13 | Compound | 32 | 632 |
| | | R05 | | |
| | 14 | Compound | 28 | 618 |
| | | R06 | | |
| | 15 | Compound | 27 | 625 |
| | | R07 | | |
| Comparative | 1 | Comparative | 15 | 530 |
| Example | | Compound 1 | | |
| | 2 | Comparative | 3 | 610 |
| | | Compound 2 | | |

According to Table 4, it is confirmed that the light-receiving devices of Examples 1 to 15 evenly had high sensitivity and high efficiency, compared to the light-receiving devices of Comparative Examples 1 and 2.

According to one or more embodiments, a light-receiving device including a condensed cyclic compound according to one or more embodiments of the present disclosure may have high sensitivity and efficiency, easy control of light-absorption wavelength, and long lifespan.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The light-receiving device, the display device, the electronic apparatus, the electronic device, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A condensed cyclic compound comprising:
at least one first repeating unit; and
at least one second repeating unit,
wherein the first repeating unit comprises at least one selected from among a first-first moiety represented by Formula 1-1 and a first-second moiety represented by Formula 1-2,
the second repeating unit comprises at least one selected from among a second-first moiety represented by Formula 2-1 and a second-second moiety represented by Formula 2-2, and
at least one of the first repeating unit further comprises at least one π electron-rich C₁-C₆₀ group: 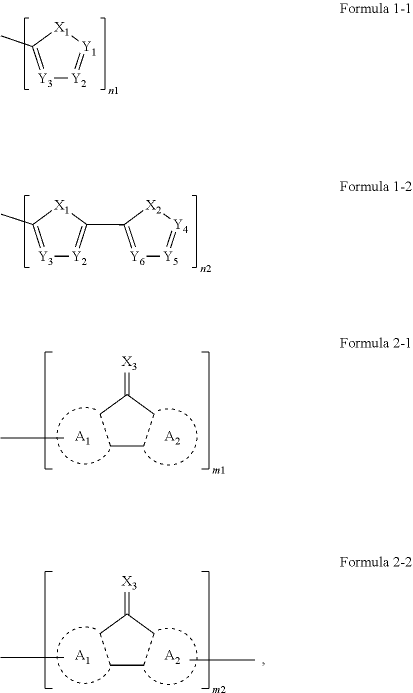
wherein, in Formulae 1-1 and 1-2,
X₁ and X₂ are each independently O, S, or Se,
Y₁ is C(R₁), CH, or C—*,
Y₂ is C(R₂) or CH,
Y₃ is C(R₃) or CH,
Y₄ is C(R₄), CH, or C—*,
Y₅ is C(R₅) or CH,
Y₆ is C(R₆) or CH,
R₁ and R₂, R₂ and R₃, R₄ and R₅, R₅ and R₆, or R₂ and R₆ are optionally bonded to each other to form: a C₃-C₆₀ carbocyclic group unsubstituted or substituted with at least one R₁₀ₐ; a C₁-C₆₀ heterocyclic group unsubstituted or substituted with at least one R₁₀ₐ; or a π electron-rich C₁-C₆₀ group unsubstituted or substituted with at least one R₁₀ₐ,
n1 and n2 are each independently an integer from 0 to 2, and a sum of n1 and n2 is 1 or more,
when n1 is 0, the first-first moiety is not included,
when n2 is 0, the first-second moiety is not included,
R₁ to R₆ and R₁₀ₐ are each independently:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a π electron-rich C₁-C₆₀ group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, or a π electron-rich C₁-C₆₀ group, each unsubstituted or substituted with deuterium, —F, a cyano group, a C₁-C₆₀ alkyl group, a C₁-C₆₀ alkoxy group, a phenyl group, a biphenyl group, or any combination thereof,
two neighboring substituents selected from among Q₁₁ to Q₁₃, Q₂₁ to Q₂₃, and Q₃₁ to Q₃₃ are optionally bonded to each other to form: a C₃-C₆₀ carbocyclic group unsubstituted or substituted with at least one R₁₀ₐ; a C₁-C₆₀ heterocyclic group unsubstituted or substituted with at least one R₁₀ₐ; or a π electron-rich C₁-C₆₀ group unsubstituted or substituted with at least one R₁₀ₐ, and
* indicates a binding site to a neighboring repeating unit, and
wherein, in Formulae 2-1 and 2-2,
X₃ is C(R₇)(R₈), C(R₇)(H), or N(R₇),
ring A₁ and ring A₂ each independently comprise: a C₃-C₆₀ carbocyclic group unsubstituted or substituted with at least one R_{10b}; or a C₁-C₆₀ heterocyclic group unsubstituted or substituted with at least one R_{10b},
m1 and m2 are each independently an integer from 0 to 2, and a sum of m1 and m2 is 1 or more,
when m1 is 0, the second-first moiety is not included,
when m2 is 0, the second-second moiety is not included,
R₇, R₈, and R_{10b} are each independently:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₄₁)(Q₄₂)(Q₄₃), —N(Q₄₁)(Q₄₂), —B(Q₄₁)(Q₄₂), —C(═O)(Q₄₁), —S(═O)₂(Q₄₁), —P(═O)(Q₄₁)(Q₄₂), or any combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, or a C₁-C₆₀ heteroarylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, —Si(Q₅₁)(Q₅₂)(Q₅₃), —N(Q₅₁)(Q₅₂), —B(Q₅₁)(Q₅₂), —C(═O)(Q₅₁), —S(═O)₂(Q₅₁), —P(═O)(Q₅₁)(Q₅₂), or any combination thereof; or
—Si(Q₆₁)(Q₆₂)(Q₆₃), —N(Q₆₁)(Q₆₂), —B(Q₆₁)(Q₆₂), —C(═O)(Q₆₁), —S(═O)₂(Q₆₁), or —P(═O)(Q₆₁)(Q₆₂),
Q₄₁ to Q₄₃, Q₅₁ to Q₅₃, and Q₆₁ to Q₆₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
two neighboring substituents selected from among Q₄₁ to Q₄₃, Q₅₁ to Q₅₃, and Q₆₁ to Q₆₃ are optionally bonded to each other to form: a C₃-C₆₀ carbocyclic group unsubstituted or substituted with at least one R_{10b}; or a C₁-C₆₀ heterocyclic group unsubstituted or substituted with at least one R_{10b}.

2. The condensed cyclic compound of claim 1, wherein n1 is 1, and n2 is 0, and
wherein one selected from among Conditions 1A to 1C is satisfied:
1A) m1 is 1, and m2 is 0;
1B) m1 is 2, and m2 is 0; and
1C)m1 is 1, and m2 is 1.

3. The condensed cyclic compound of claim 1, wherein n1 is 0, and n2 is 1, and
wherein one selected from among Conditions 2A to 2C is satisfied:
2A) m1 is 1, and m2 is 0;
2B) m1 is 2, and m2 is 0; and
2C) m1 is 1, and m2 is 1.

4. The condensed cyclic compound of claim 1, wherein n1 is 2, and n2 is 0, and
wherein one selected from among Conditions 3A to 3C is satisfied:
3A) m1 is 0, and m2 is 1;
3B) m1 is 0, and m2 is 2; and
3C) m1 is 1, and m2 is 1.

5. The condensed cyclic compound of claim 1, wherein n1 is 0, and n2 is 2, and
wherein one selected from among Conditions 4A to 4E is satisfied:
4A) m1 is 1, and m2 is 0;
4B) m1 is 2, and m2 is 0;
4C) m1 is 0, and m2 is 1;
4D) m1 is 0, and m2 is 2; and
4E) m1 is 1, and m2 is 1.

6. The condensed cyclic compound of claim 1, wherein n1 is 1, and n2 is 1, and
wherein one selected from among Conditions 5A to 5E is satisfied:
5A) m1 is 1, and m2 is 0;
5B) m1 is 2, and m2 is 0;
5C) m1 is 0, and m2 is 1;
5D) m1 is 0, and m2 is 2; and
5E) m1 is 1, and m2 is 1.

7. The condensed cyclic compound of claim 1, wherein,
when the sum of m1 and m2 is 1, X₃ is C(CN)(CN), C(CN)H, or N(CN), and
when the sum of m1 and m2 is 2 or more, each of the X₃ in the second-first moiety or the X₃ in the second-second moiety is C(CN)(CN), C(CN)H, or N(CN).

8. The condensed cyclic compound of claim 1, wherein ring A₁ and ring A₂ are each independently a 5-membered cyclic group, a 5-membered heterocyclic group, a 6-membered cyclic group, a 6-membered heterocyclic group, a polycyclic C₆-C₃₀ cyclic group comprising a 5-membered cyclic group, a polycyclic C₇-C₃₀ cyclic group comprising a 6-membered cyclic group, a polycyclic C₅-C₃₀ heterocyclic group comprising a 5-membered cyclic group, a polycyclic C₆-C₃₀ heterocyclic group comprising a 6-membered cyclic group, a polycyclic C₁-C₃₀ heterocyclic group comprising a 5-membered heterocyclic group, or a polycyclic C₁-C₃₀ heterocyclic group comprising a 6-membered heterocyclic group.

9. The condensed cyclic compound of claim 1, wherein the π electron-rich C₁-C₆₀ group comprises at least one selected from among *—C(Q₁)(Q₂)-*, a π electron-rich C₃-C₆₀ cyclic group, and groups represented by Formulae 3-1 to 3-3: 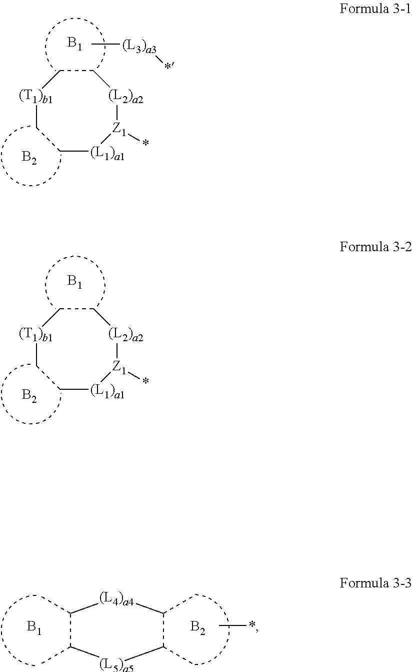
and
wherein, in Formulae 3-1 to 3-3,
Z₁ is C(R₉), N, or P,
ring B₁ and ring B₂ each independently comprise: a C₃-C₆₀ carbocyclic group unsubstituted or substituted with at least one R₁₀ₐ; or a C₁-C₆₀ heterocyclic group unsubstituted or substituted with at least one R₁₀ₐ,
L₁ to L₅ are each independently *—O*′, *—S—*′, *—C(Q₁)(Q₂)-*′, *—C(Q₁)═*′, *═C(Q₁)-* *—C(Q₁)=C(Q₂)-*′, *—C≡C—*′, *—N(Q₁)-*′, *—P(Q₁)-*′, *—Si(Q₁)(Q₂)-*′, *—P(Q₁)(Q₂)-*′, or *—Ge(Q₁)(Q₂)-*′,
a1 to a5 are each independently an integer from 0 to 5,
T₁ is a single bond, *—O—*′, *—S—*′, *—C(Q₁)(Q₂)-*′, *—C(Q₁)═*′, *═C(Q₁)-*′, *—C(Q₁)═C(Q₂)-*′, *—C≡C—* *—N(Q₁)-*, *—P(Q₁)-*, *—Si(Q₁)(Q₂)-*′, *—P(Q₁)(Q₂)-*′, or *—Ge(Q₁)(Q₂)-*′,
b1 is an integer from 0 to 5,
when a1 is 0, *-(L₁)ₐ₁-*′ is a single bond,
when a2 is 0, *-(L₂)ₐ₂-*′ is a single bond,
when a3 is 0, *-(L₃)ₐ₃-*′ is a single bond,
when a4 is 0, *-(L₄)ₐ₄-*′ is a single bond,
when a5 is 0, *-(L₅)ₐ₅-*′ is a single bond,
when b1 is 0, ring B₁ and ring B₂ are not directly linked to each other,
Q₁ and Q₂ respectively correspond to Q₁₁ and Q₁₂ of Formulae 1-1 and 1-2, and
* and *′ each indicate a linking site to the first repeating unit.

10. A condensed cyclic compound comprising:
at least one electron-accepting moiety; and
at least one spacer moiety,
wherein the electron-accepting moiety comprises a 9-methylenefluorene moiety and at least one cyano group,
the spacer moiety comprises at least one of a thiophene moiety or a selenophene moiety,
the spacer moiety further comprises at least one π electron-rich C₁-C₆₀ group, and
the π electron-rich C₁-C₆₀ group is not directly linked to the electron-accepting moiety.

11. The condensed cyclic compound of claim 1, wherein a lowest unoccupied molecular orbital (LUMO) energy level of the condensed cyclic compound is less than −2.5 eV, and a highest occupied molecular orbital (HOMO) energy level of the condensed cyclic compound is less than −4.5 eV.

12. The condensed cyclic compound of claim 1, wherein the condensed cyclic compound is to absorb green light or red light.

13. The condensed cyclic compound of claim 1, wherein the condensed cyclic compound is one selected from among Compounds 1 to 48 and R01 to R07:

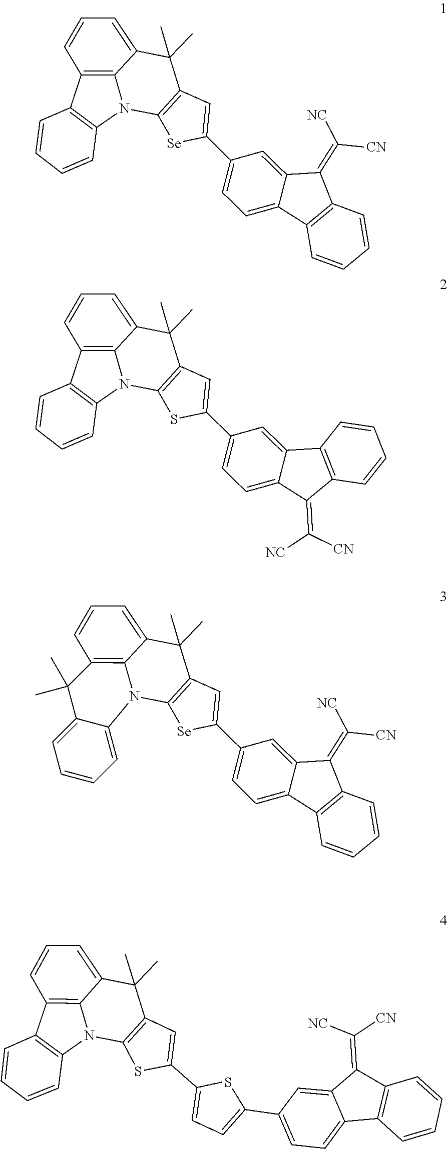 [SMILES: CC1(C)c2cc(-c3ccc4c(c3)C(=C(C#N)C#N)c3ccccc3-4)[se]c2-n2c3ccccc3c3cccc1c32 ; CC1(C)c2cc(-c3ccc4c(c3)-c3ccccc3C4=C(C#N)C#N)sc2-n2c3ccccc3c3cccc1c32 ; CC1(C)c2ccccc2N2c3[se]c(-c4ccc5c(c4)C(=C(C#N)C#N)c4ccccc4-5)cc3C(C)(C)c3cccc1c32 ; CC1(C)c2cc(-c3ccc(-c4ccc5c(c4)C(=C(C#N)C#N)c4ccccc4-5)s3)sc2-n2c3ccccc3c3cccc1c32]

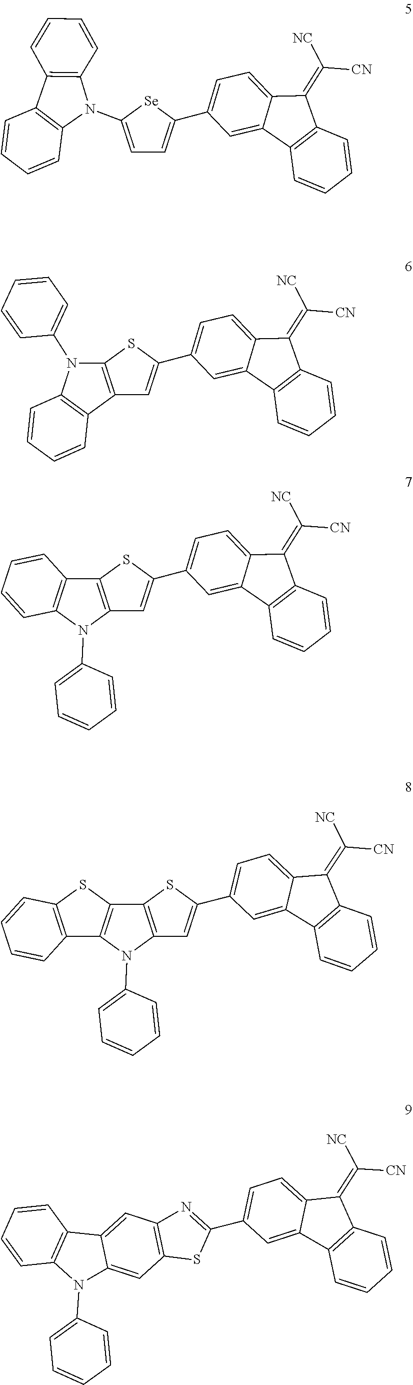 [SMILES: N#CC(C#N)=C1c2ccccc2-c2cc(-c3ccc(-n4c5ccccc5c5ccccc54)[se]3)ccc21 ; N#CC(C#N)=C1c2ccccc2-c2cc(-c3cc4c5ccccc5n(-c5ccccc5)c4s3)ccc21 ; N#CC(C#N)=C1c2ccccc2-c2cc(-c3cc4c(s3)c3ccccc3n4-c3ccccc3)ccc21 ; N#CC(C#N)=C1c2ccccc2-c2cc(-c3cc4c(s3)c3sc5ccccc5c3n4-c3ccccc3)ccc21 ; N#CC(C#N)=C1c2ccccc2-c2cc(-c3nc4cc5c6ccccc6n(-c6ccccc6)c5cc4s3)ccc21]

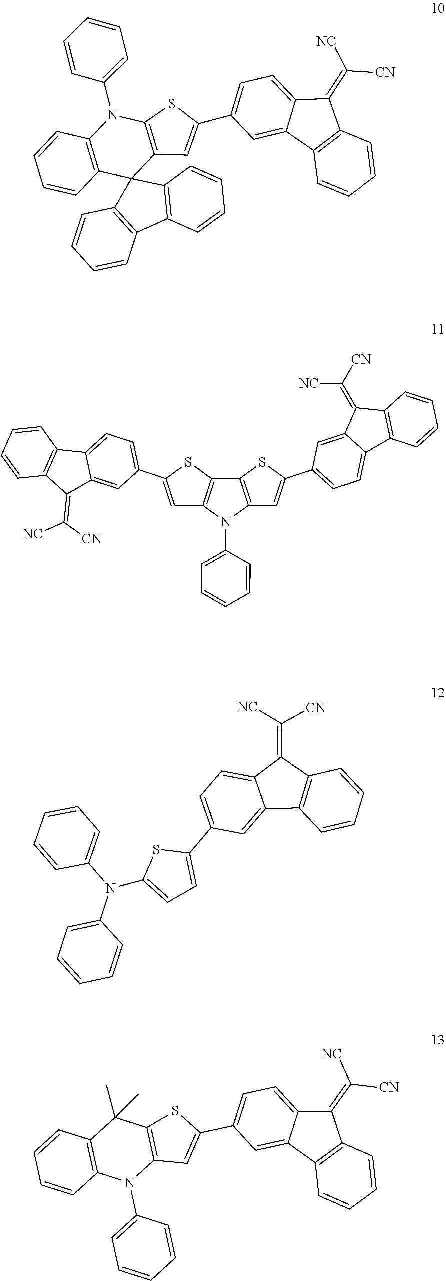 [SMILES: N#CC(C#N)=C1c2ccccc2-c2cc(-c3cc4c(s3)N(c3ccccc3)c3ccccc3C43c4ccccc4-c4ccccc43)ccc21 ; N#CC(C#N)=C1c2ccccc2-c2ccc(-c3cc4c(s3)c3sc(-c5ccc6c(c5)C(=C(C#N)C#N)c5ccccc5-6)cc3n4-c3ccccc3)cc21 ; N#CC(C#N)=C1c2ccccc2-c2cc(-c3ccc(N(c4ccccc4)c4ccccc4)s3)ccc21 ; CC1(C)c2ccccc2N(c2ccccc2)c2cc(-c3ccc4c(c3)-c3ccccc3C4=C(C#N)C#N)sc21]

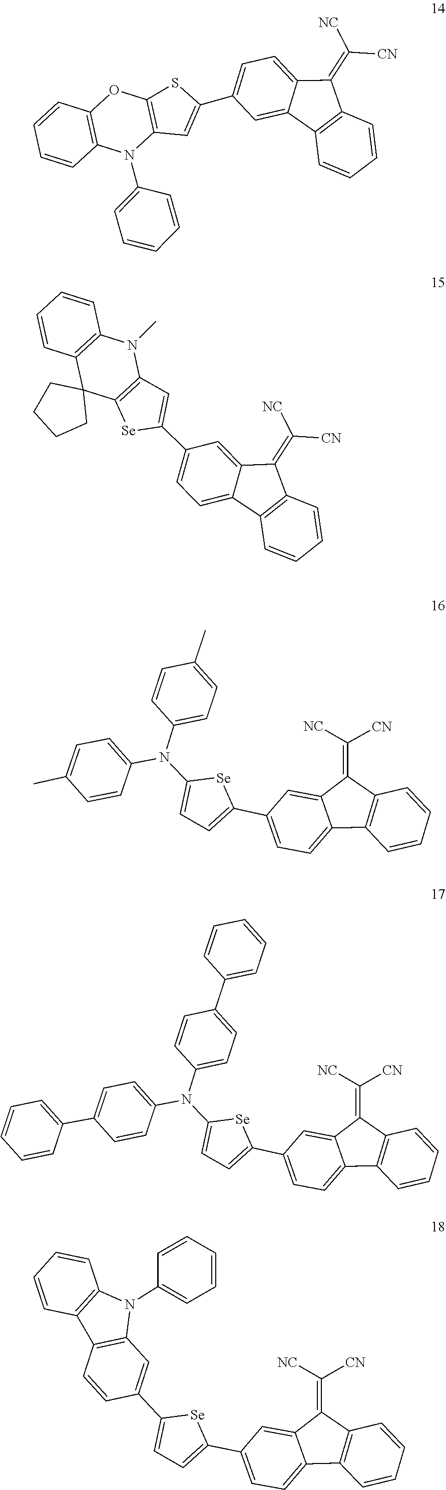 [SMILES: N#CC(C#N)=C1c2ccccc2-c2cc(-c3cc4c(s3)Oc3ccccc3N4c3ccccc3)ccc21 ; CN1c2ccccc2C2(CCCC2)c2[se]c(-c3ccc4c(c3)C(=C(C#N)C#N)c3ccccc3-4)cc21 ; Cc1ccc(N(c2ccc(C)cc2)c2ccc(-c3ccc4c(c3)C(=C(C#N)C#N)c3ccccc3-4)[se]2)cc1 ; N#CC(C#N)=C1c2ccccc2-c2ccc(-c3ccc(N(c4ccc(-c5ccccc5)cc4)c4ccc(-c5ccccc5)cc4)[se]3)cc21 ; N#CC(C#N)=C1c2ccccc2-c2ccc(-c3ccc(-c4ccc5c6ccccc6n(-c6ccccc6)c5c4)[se]3)cc21]

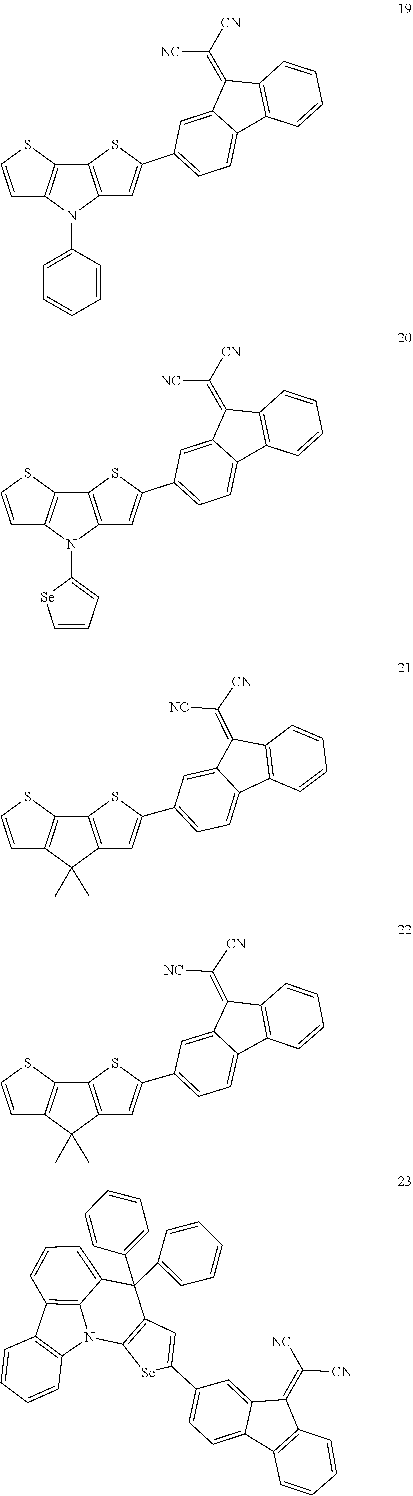

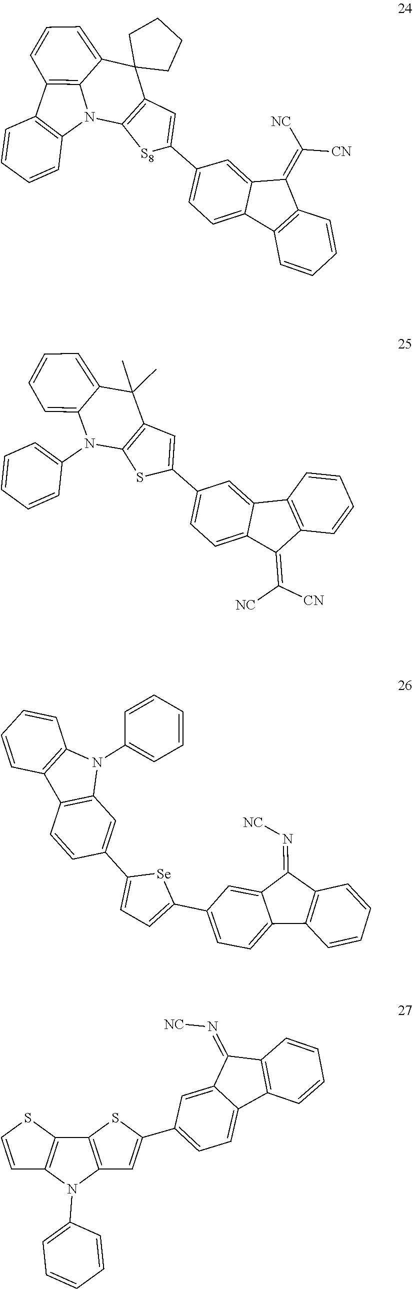

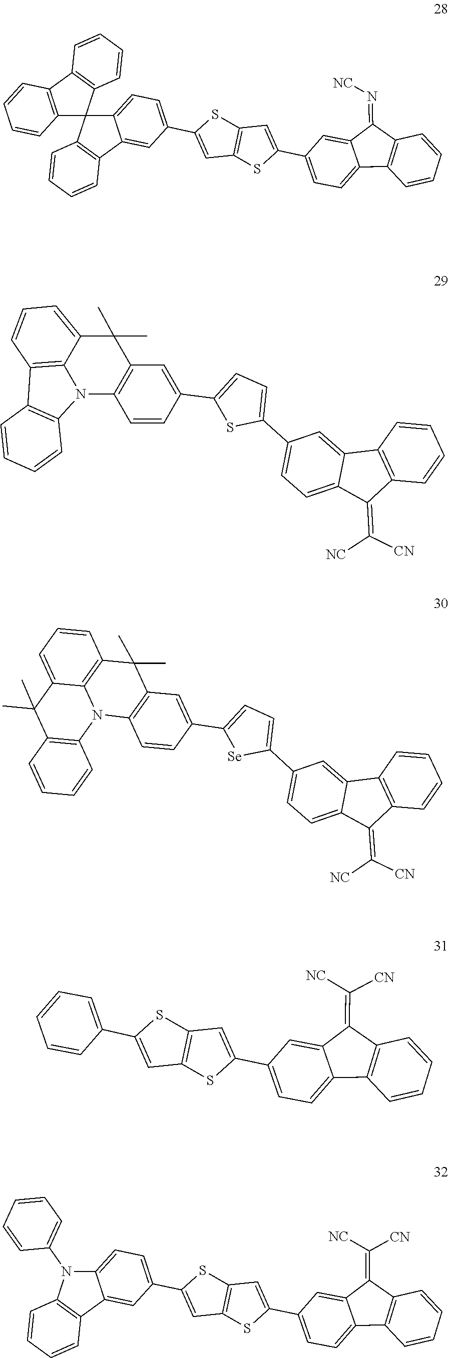 [SMILES: N#C/N=C1/c2ccccc2-c2ccc(-c3cc4sc(-c5ccc6c(c5)-c5ccccc5C65c6ccccc6-c6ccccc65)cc4s3)cc21 ; CC1(C)c2cc(-c3ccc(-c4ccc5c(c4)-c4ccccc4C5=C(C#N)C#N)s3)ccc2-n2c3ccccc3c3cccc1c32 ; CC1(C)c2ccccc2N2c3ccc(-c4ccc(-c5ccc6c(c5)-c5ccccc5C6=C(C#N)C#N)[se]4)cc3C(C)(C)c3cccc1c32 ; N#CC(C#N)=C1c2ccccc2-c2ccc(-c3cc4sc(-c5ccccc5)cc4s3)cc21 ; N#CC(C#N)=C1c2ccccc2-c2ccc(-c3cc4sc(-c5ccc6c(c5)c5ccccc5n6-c5ccccc5)cc4s3)cc21]

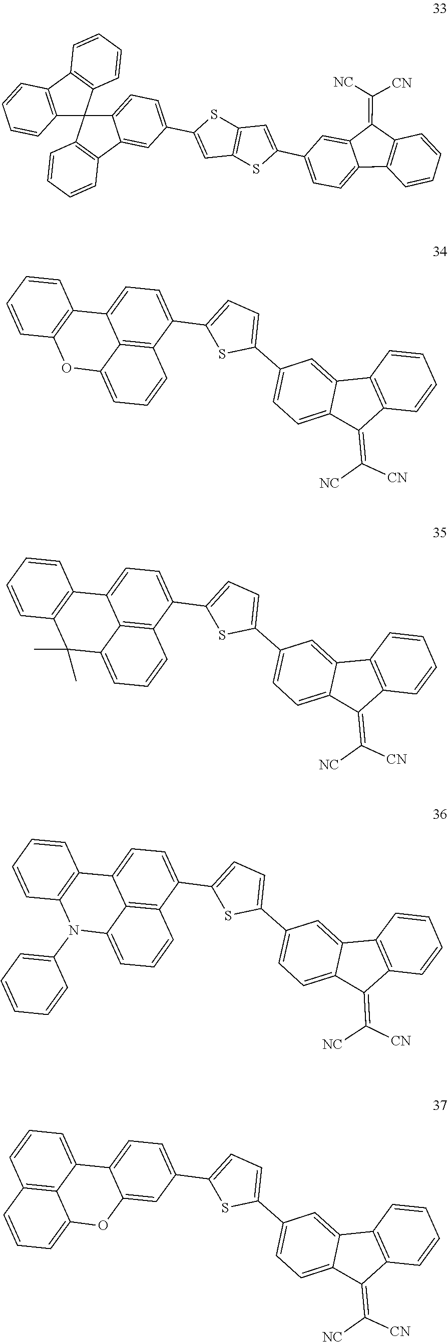 [SMILES: N#CC(C#N)=C1c2ccccc2-c2ccc(-c3cc4sc(-c5ccc6c(c5)-c5ccccc5C65c6ccccc6-c6ccccc65)cc4s3)cc21 ; N#CC(C#N)=C1c2ccccc2-c2cc(-c3ccc(-c4ccc5c6c(cccc46)Oc4ccccc4-5)s3)ccc21 ; CC1(C)c2ccccc2-c2ccc(-c3ccc(-c4ccc5c(c4)-c4ccccc4C5=C(C#N)C#N)s3)c3cccc1c23 ; N#CC(C#N)=C1c2ccccc2-c2cc(-c3ccc(-c4ccc5c6c(cccc46)N(c4ccccc4)c4ccccc4-5)s3)ccc21 ; N#CC(C#N)=C1c2ccccc2-c2cc(-c3ccc(-c4ccc5c(c4)Oc4cccc6cccc-5c46)s3)ccc21]

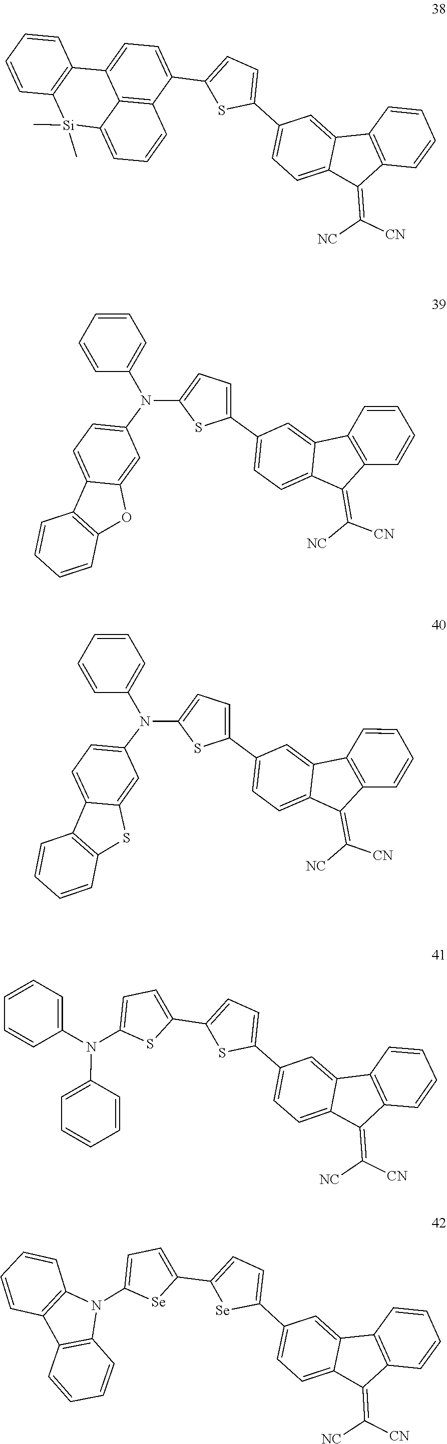 [SMILES: C[Si]1(C)c2ccccc2-c2ccc(-c3ccc(-c4ccc5c(c4)-c4ccccc4C5=C(C#N)C#N)s3)c3cccc1c23 ; N#CC(C#N)=C1c2ccccc2-c2cc(-c3ccc(N(c4ccccc4)c4ccc5c(c4)oc4ccccc45)s3)ccc21 ; N#CC(C#N)=C1c2ccccc2-c2cc(-c3ccc(N(c4ccccc4)c4ccc5c(c4)sc4ccccc45)s3)ccc21 ; N#CC(C#N)=C1c2ccccc2-c2cc(-c3ccc(-c4ccc(N(c5ccccc5)c5ccccc5)s4)s3)ccc21 ; N#CC(C#N)=C1c2ccccc2-c2cc(-c3ccc(-c4ccc(-n5c6ccccc6c6ccccc65)[se]4)[se]3)ccc21]

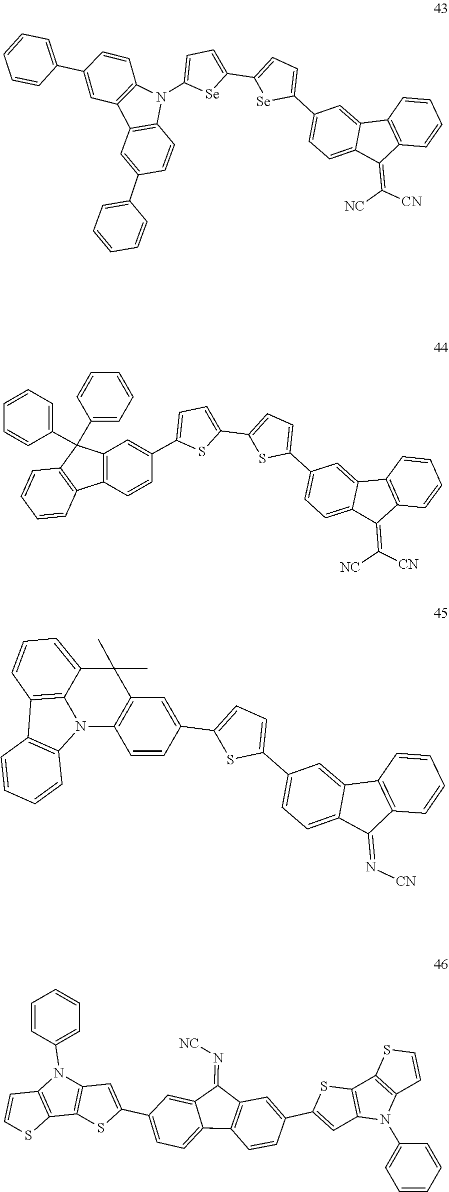 [SMILES: N#CC(C#N)=C1c2ccccc2-c2cc(-c3ccc(-c4ccc(-n5c6ccc(-c7ccccc7)cc6c6cc(-c7ccccc7)ccc65)[se]4)[se]3)ccc21 ; N#CC(C#N)=C1c2ccccc2-c2cc(-c3ccc(-c4ccc(-c5ccc6c(c5)C(c5ccccc5)(c5ccccc5)c5ccccc5-6)s4)s3)ccc21 ; CC1(C)c2cc(-c3ccc(-c4ccc5c(c4)-c4ccccc4/C5=N\C#N)s3)ccc2-n2c3ccccc3c3cccc1c32 ; N#CN=C1c2cc(-c3cc4c(s3)c3sccc3n4-c3ccccc3)ccc2-c2ccc(-c3cc4c(s3)c3sccc3n4-c3ccccc3)cc21]

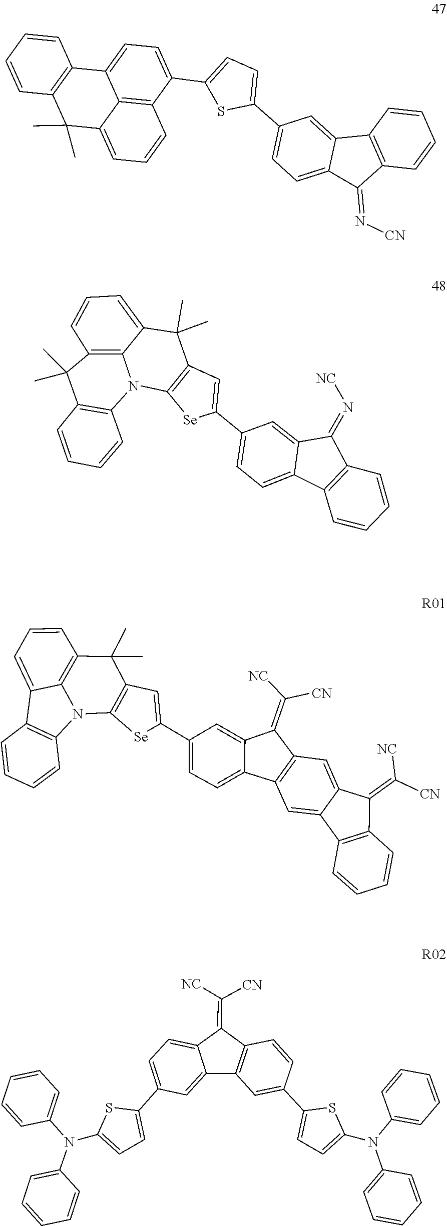 [SMILES: CC1(C)c2ccccc2-c2ccc(-c3ccc(-c4ccc5c(c4)-c4ccccc4/C5=N\C#N)s3)c3cccc1c23 ; CC1(C)c2ccccc2N2c3[se]c(-c4ccc5c(c4)/C(=N\C#N)c4ccccc4-5)cc3C(C)(C)c3cccc1c32 ; CC1(C)c2cc(-c3ccc4c(c3)C(=C(C#N)C#N)c3cc5c(cc3-4)-c3ccccc3C5=C(C#N)C#N)[se]c2-n2c3ccccc3c3cccc1c32 ; N#CC(C#N)=C1c2ccc(-c3ccc(N(c4ccccc4)c4ccccc4)s3)cc2-c2cc(-c3ccc(N(c4ccccc4)c4ccccc4)s3)ccc21]

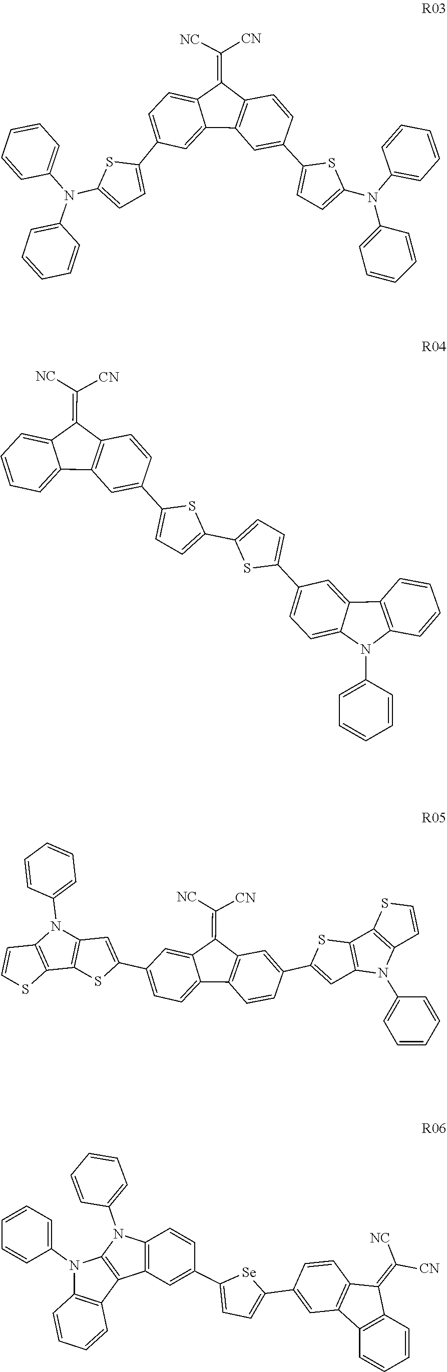 [SMILES: N#CC(C#N)=C1c2ccc(-c3ccc(N(c4ccccc4)c4ccccc4)s3)cc2-c2cc(-c3ccc(N(c4ccccc4)c4ccccc4)s3)ccc21 ; N#CC(C#N)=C1c2ccccc2-c2cc(-c3ccc(-c4ccc(-c5ccc6c(c5)c5ccccc5n6-c5ccccc5)s4)s3)ccc21 ; N#CC(C#N)=C1c2cc(-c3cc4c(s3)c3sccc3n4-c3ccccc3)ccc2-c2ccc(-c3cc4c(s3)c3sccc3n4-c3ccccc3)cc21 ; N#CC(C#N)=C1c2ccccc2-c2cc(-c3ccc(-c4ccc5c(c4)c4c6ccccc6n(-c6ccccc6)c4n5-c4ccccc4)[se]3)ccc21]

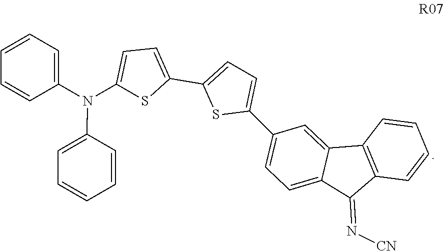 [SMILES: N#C/N=C1\c2ccccc2-c2cc(-c3ccc(-c4ccc(N(c5ccccc5)c5ccccc5)s4)s3)ccc21]

14. A light-receiving device comprising:
a first electrode;
a second electrode facing the first electrode;
an interlayer between the first electrode and the second electrode and comprising a light-receiving layer; and
the condensed cyclic compound of claim 1.

15. The light-receiving device of claim 14, wherein,
the first electrode is an anode,
the second electrode is a cathode, and
the interlayer further comprises:
i) a hole transport region between the first electrode and the light-receiving layer and comprising a hole injection layer, a hole transport layer, an auxiliary layer, an electron-blocking layer, or any combination thereof; and
ii) an electron transport region between the light-receiving layer and the second electrode and comprising a hole-blocking layer, an electron transport layer, an electron injection layer, or any combination thereof.

16. The light-receiving device of claim 14, wherein the light-receiving layer comprises the condensed cyclic compound.

17. The light-receiving device of claim 14, wherein the light-receiving layer is to absorb green light or red light.

18. An electronic apparatus comprising the light-receiving device of claim 14.

19. The electronic apparatus of claim 18, further comprising a thin-film transistor,
wherein,
the thin-film transistor comprises a source electrode and a drain electrode, and
the first electrode of the light-receiving device is electrically connected to the source electrode or the drain electrode of the thin-film transistor.

20. An electronic device comprising the light-receiving device of claim 14, wherein the electronic device is at least one selected from a flat panel display, a curved display, a computer monitor, a medical monitor, a television, a billboard, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (PDA), a biometric authentication device, a wearable device, a laptop computer, a digital camera, a camcorder, a viewfinder, a microdisplay, a three-dimensional (3D) display, a virtual reality or augmented reality display, a vehicle, a video wall with multiple displays tiled together, a theater or stadium screen, a phototherapy device, and a signboard.
